(19) United States
(12) Patent Application Publication
(10) Pub. No.: US 2026/0355184 A1
(43) Pub. Date: Nov. 12, 2026

(54) SMALL MOLECULE DEGRADERS OF HIV-1 TAT PROTEIN

(71) Applicant: University of Florida Research Foundation, Incorporated, Gainesville, FL (US)

(72) Inventors: Susana Tavares Valente, Gainesville, FL (US); Sonia Mediouni Jablonski, Gainesville, FL (US); Joseph Jablonski, Gainesville, FL (US)

(73) Assignee: University of Florida Research Foundation, Incorporated, Gainesville, FL (US)

(21) Appl. No.: 19/115,036

(22) PCT Filed: Sep. 27, 2023
(86) PCT No.: PCT/US2023/075244

§ 371 Date: Mar. 25, 2025

Related U.S. Application Data
(60) Provisional application No. 63/410,614, filed on Sep. 27, 2022.

(51) Int. Cl. A61K 31/498; A61K 31/404; A61K 31/4155; A61K 31/416; A61K 31/4162; A61K 31/4178; A61K 31/4184; A61K 31/4196; A61K 31/437; A61K 31/4406; A61K 31/4412; A61K 31/4433; A61K 31/4439; A61K 31/4462; A61K 31/454; A61K 31/4709; A61K 31/496; A61K 31/502; A61K 31/519; A61K 31/5377; A61K 45/06; A61P 31/18; C07D 209/42; C07D 211/26; C07D 213/64; C07D 213/75; C07D 213/85; C07D 233/61; C07D 233/64; C07D 235/06; C07D 249/08; C07D 295/13; C07D 401/12; C07D 403/06; C07D 403/12; C07D 405/12; C07D 413/06; C07D 417/12; C07D 471/04; C07D 487/04; C07D 491/113

(52) U.S. Cl. CPC A61K 31/498; A61K 31/404; A61K 31/4155; A61K 31/416; A61K 31/4162; A61K 31/4178; A61K 31/4184; A61K 31/4196; A61K 31/437; A61K 31/4406; A61K 31/4412; A61K 31/4433; A61K 31/4439; A61K 31/4462; A61K 31/454; A61K 31/4709; A61K 31/496; A61K 31/502; A61K 31/519; A61K 31/5377; A61K 45/06; A61P 31/18; C07D 209/42; C07D 211/26; C07D 213/64; C07D 213/75; C07D 213/85; C07D 233/61; C07D 233/64; C07D 235/06; C07D 249/08; C07D 295/13; C07D 401/12; C07D 403/06; C07D 403/12; C07D 405/12; C07D 413/06; C07D 417/12; C07D 471/04; C07D 487/04; C07D 491/113

(57) ABSTRACT

The present disclosure provides compounds of Formulae (I), (II), and (III), and pharmaceutically acceptable salts, solvates, hydrates, polymorphs, co-crystals, tautomers, stereoisomers, isotopically labeled compounds, and prodrugs thereof, which may promote the degradation of human immunodeficiency virus type 1 (HIV-1) Tat protein. The present disclosure also provides pharmaceutical compositions and kits comprising the compounds, or pharmaceutically acceptable salts, solvates, hydrates, polymorphs, cocrystals, tautomers, stereoisomers, isotopically labeled compounds, or prodrugs thereof, and methods of treating or preventing diseases (e.g., HIV) by administering to a subject in need thereof the compounds, or pharmaceutically acceptable salts, solvates, hydrates, polymorphs, co-crystals, tautomers, stereoisomers, isotopically labeled compounds, or prodrugs thereof, or pharmaceutical compositions thereof.

### RELATED APPLICATIONS

The present application claims priority under 35 U.S.C. § 119(e) to U.S. provisional application, U.S. Ser. No. 63/410,614, filed Sep. 27, 2022, which is incorporated herein by reference.

### GOVERNMENT SUPPORT

This invention was made with government support under Grant No. R21 AI158296 awarded by the National Institutes of Health. The government has certain rights in the invention.

### BACKGROUND

Treatment of people living with HIV (PLWH) with antiretroviral drugs (ARVs) has extended their quality and expectancy of life; however, it has been unsuccessful in curing HIV infection. ARVs fall into the following major classes: fusion inhibitors (FIs), nucleoside/nucleotide reverse transcriptase inhibitors (NRTIs), non-nucleoside reverse transcriptase inhibitors (NNRTIs), protease inhibitors (PIs) and integrase inhibitors (INIs). Antiretroviral therapy (ART) against HIV is based on triple or quadruple combinations of ARVs^{1,2}, and despite effective suppression of HIV replication to below the detection limit, upon cell stimulation or treatment interruption, latent proviruses can reinitiate viral production³⁻⁵. Elimination of this latent reservoir is a major goal of HIV eradication strategies^{6,7}; however, approaches such as shock-and-kill have not been successful so far⁸, and significant efforts are being invested by laboratories worldwide to find a functional cure of HIV remission. A functional cure entails long-term, durable control of viral expression in the absence of therapy, thus preventing disease progression and transmission, despite the presence of integrated proviruses. Without wishing to be bound by any particular theory, one such strategy, the block-and-lock approach, is based on the premise that transcriptional inhibitors may mediate epigenetic silencing of proviral expression, locking the virus in a profound state of latency, from which reactivation is very unlikely to occur upon ART interruption.

This principle was demonstrated with the small molecule didehydro-Cortistatin A (dCA). In CD4⁺T cells isolated from infected individuals and in the humanized mouse model of HIV latency⁹⁻¹¹, combining dCA with ART inhibits transcription and blocks viral rebound upon treatment interruption¹². dCA targets the viral Tat protein, which plays a role in regulating the transition between the latent and active transcriptional states. This potent activator of HIV gene expression is involved in the synthesis of full-length transcripts of the integrated viral genome by RNA polymerase II (RNAPII)¹³⁻¹⁵. Tat mediates association between the host positive transcription elongation factor (P-TEFb) complex and the transactivation-responsive (TAR) element of the nascent viral RNA to promote transcriptional elongation from the viral promoter (FIG. 1A)¹⁶⁻¹⁸. Specifically, Tat binds the 50-terminal region of HIV mRNA's stem-bulge loop structure, the TAR element, to activate transcription. Since Tat resides under the control of the same promoter, it enhances its own transcription via a positive feedback loop.

Tat is a target for therapeutic intervention because 1) it is expressed early during virus replication; 2) it has no host cellular homologs; 3) Tat inhibitors block the positive transactivation feedback loop critical for viral activation; and 4) in breaking this feedback loop, epigenetic modifications accumulate at the HIV promoter, rendering reactivation less likely. Tat is also notorious for its role in neurotoxicity, blood-brain barrier disruption and neuroinflammation¹⁹.

Thus, the development of Tat inhibitors, such as dCA, could complement antiretroviral therapy (ART) and silence virus outgrowth from HIV tissue reservoirs in people living with HIV (PLWH). However, the cost of producing large quantities of this complex molecule is a major hurdle towards advancing dCA into clinical trials²⁰. Although efforts towards a cost-effective synthesis pathway and cheaper analogs are underway²¹, additional clinical candidates, structurally distinct from dCA, that are more readily available yet still provide equivalent bioactivity, are needed in the pre-clinical pipeline.

### SUMMARY

This disclosure is based in-part on the Applicant's discovery of novel compounds that may promote the degradation of human immunodeficiency virus type 1 (HIV-1) Tat protein.

In certain aspects, provided herein are compounds of Formula (I):

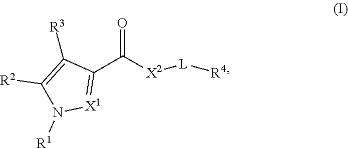

or pharmaceutically acceptable salts, solvates, hydrates, polymorphs, co-crystals, tautomers, stereoisomers, isotopically labeled compounds, or prodrugs thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R² and R³ are independently hydrogen, halogen, substituted or unsubstituted carbocyclyl, substituted or unsubstituted heterocyclyl, substituted or unsubstituted aryl, or substituted or unsubstituted heteroaryl, or R² and R³, together with the atoms to which they are attached, are joined together to form a carbocyclic, heterocyclic, aryl, or heteroaryl ring, each of which may be optionally substituted;
L is substituted or unsubstituted alkylene or heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In some aspects, the compounds of Formula (I) are of Formula (I-g):

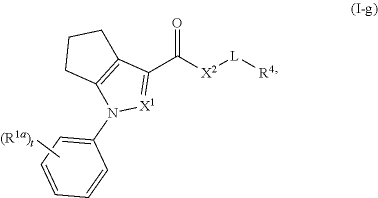

or pharmaceutically acceptable salts, solvates, hydrates, polymorphs, co-crystals, tautomers, stereoisomers, isotopically labeled compounds, or prodrugs thereof, wherein:
L is substituted or unsubstituted alkylene, or substituted or unsubstituted heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
R^{1a} is hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; and
t is 1, 2, 3, 4, or 5.

Further disclosed herein are compounds of Formula (II):

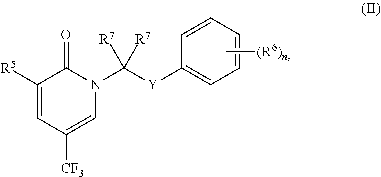

or pharmaceutically acceptable salts, solvates, hydrates, polymorphs, co-crystals, tautomers, stereoisomers, isotopically labeled compounds, or prodrugs thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, hydroxy, optionally substituted alkoxy, substituted or unsubstituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, carbocyclyl, substituted or unsubstituted heterocyclyl, substituted or unsubstituted aryl, or substituted or unsubstituted heteroaryl;
Y is arylene, heteroarylene, —N(R^{d})C(═O)— or —C(═O)N(R^{d})—;
R^{d} is hydrogen, or substituted or unsubstituted alkyl; and
n is 1, 2, 3, 4, or 5.

In certain aspects, the compounds of Formula (II) are of Formula (II-c):

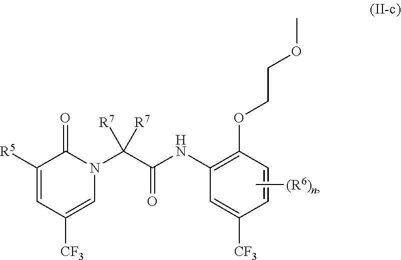

or pharmaceutically acceptable salts, solvates, hydrates, polymorphs, co-crystals, tautomers, stereoisomers, isotopically labeled compounds, or prodrugs thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, a optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and
n is 1, 2, or 3.

Also disclosed herein are compounds of Formula (III′):

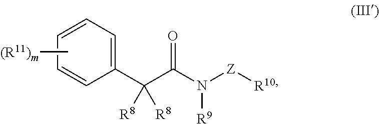

or a pharmaceutically acceptable salt, solvate, hydrate, polymorph, co-crystal, tautomer, stereoisomer, isotopically labeled compound, or prodrug thereof, wherein:
each R¹¹ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁸ is independently hydrogen, halogen, hydroxy, —N(R^{e})₂— substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
or one instance of R⁸ and R¹¹ is joined together with the atoms to which they are attached to form a 5 or 6 membered substituted or unsubstituted carbocyclic, substituted or unsubstituted heterocyclic, or substituted or unsubstituted heteroaryl ring;
each instance of R^{e} is independently hydrogen, substituted or unsubstituted alkyl, or substituted or unsubstituted acyl;
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
or one instance of R⁸ and R⁹ is joined together with the atoms to which they are attached to form a substituted or unsubstituted 5 membered heterocyclic ring;
R¹⁰ is substituted or unsubstituted carbocyclyl, substituted or unsubstituted heterocyclyl, substituted or unsubstituted aryl, or substituted or unsubstituted heteroaryl;
Z is substituted or unsubstituted alkylene, substituted or unsubstituted heteroalkylene, or substituted or unsubstituted heteroarylene; and
m is 1, 2, 3, 4, or 5.

Further disclosed herein are compounds of Formula (III):

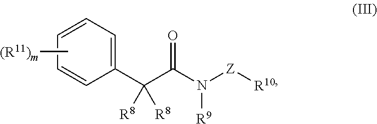

or pharmaceutically acceptable salts, solvates, hydrates, polymorphs, co-crystals, tautomers, stereoisomers, isotopically labeled compounds, or prodrugs thereof, wherein:
each R¹¹ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁸ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
R¹⁰ is substituted or unsubstituted carbocyclyl, substituted or unsubstituted heterocyclyl, substituted or unsubstituted aryl, or substituted or unsubstituted heteroaryl;
Z is substituted or unsubstituted alkylene, or substituted or unsubstituted heteroalkylene; and
m is 1, 2, 3, 4, or 5.

In some aspects, the compounds of Formula (III) are of Formula (III-b):

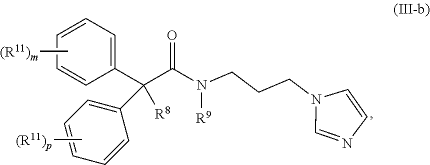

or pharmaceutically acceptable salts, solvates, hydrates, polymorphs, co-crystals, tautomers, stereoisomers, isotopically labeled compounds, or prodrugs thereof, wherein:
each R¹¹ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁸ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
m is 1, 2, 3, 4, or 5.

In some aspects, the compounds of any of the formulae herein (e.g., Formulae (I), (II), or (III)), or pharmaceutically acceptable salts, solvates, hydrates, polymorphs, co-crystals, tautomers, stereoisomers, isotopically labeled compounds, or prodrugs thereof may bind preferentially to Tat residue Y26.

Further disclosed herein are pharmaceutical compositions comprising the compounds of any of the formulae herein (e.g., Formulae (I), (II), or (III)), or pharmaceutically acceptable salts, solvates, hydrates, polymorphs, co-crystals, tautomers, stereoisomers, isotopically labeled compounds, or prodrugs thereof as described herein, and an excipient.

In some aspects, disclosed herein are methods of treating or preventing a disease (e.g., a viral disease (e.g., HIV)) in a subject in need thereof, comprising administering to the subject in need thereof a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or a pharmaceutical composition thereof as described herein.

Further disclosed herein are methods of promoting the degradation of HIV-1 Tat protein in a subject in need thereof or in a cell, tissue, or biological sample, comprising administering to the subject in need thereof or contacting the cell, tissue, or biological sample with an effective amount of a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or a pharmaceutical composition thereof as described herein; wherein the cell, tissue, or biological sample is in vivo or in vitro.

Further provided herein are kits comprising a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or a pharmaceutical composition thereof as described herein, and instructions for using the compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or the pharmaceutical composition thereof as described herein.

### BRIEF DESCRIPTION OF THE DRAWINGS

FIG. 1. HIV transcription is primary regulated by the HIV Tat protein. A. Domains of HIV Tat protein. B. HIV transcription initiation and elongation. (1) Formation of the pre-initiation complex, including TFIIH, upon binding of transcription factors to the HIV promoter (such as NF-kB and SP1) and recruitment of RNA polymerase II (RNAPII). The subunit CDK7 of TFIIH phosphorylates RNAPII C-terminal domain (CTD) at Ser5 to allow initiation of transcription. (2) RNAPII stalls shortly after transcribing TAR RNA. Tat recruits P-TEFb (CDK9/Cyclin T1) to TAR and CDK9 phosphorylates RNAPII CTD at Ser2, as well as the negative elongation factor (NELF) and DSIF. DSIF is converted to an elongation factor while NELF is removed from the transcription complex. (3) These phosphorylation events and remodeling of Nucleosome 1 (Nuc-1) allows productive elongation and full-length viral mRNA production.

FIG. 2. Identification of 3 HIV Tat inhibitors. A. Schematic of the Tat-TAR assay. B. Schematic of the HTS. Assay 1: Tat-TAR assay, Assay 2: TNF-α activation, Assay 3: Toxicity assay. C. Structure of the lead hits. D. Table summarizing lead hits characteristics and HTS results. TI: therapeutic index. E. Tat-TAR assay using HIV Tat 86 a.a from different HIV clades (n=1).

FIG. 3. TT-44881, TT-44856 and TT-44863 inhibit HIV acute and latent infections. A. Impact of the 3 lead hits on HIV acute infection. TI: therapeutic index. B. Schematic depicting the characteristics of OM10.1 and ACH2 cells. C. Impact of the lead hits on different models of HIV latency. Shown is representative of n=3. ARVs: antiretrovirals: 100 nM Efavirenz, 200 nM Lamivudine and 200 nM Raltegravir.

FIG. 4. TT-44881 and TT-44863 inhibit HIV transcription and block reactivation from latency independently of the Tat-TAR axis. A. TT-44881 and TT-44863 inhibit HIV transcription in OM10.1 cells over a 96-day period and SAHA mediated reactivation from latency on day 12 and day 32. Shown is n=1. B. TT-44881 and TT-44863 inhibit HIV transcription in ACH2 cells over a period of 96 days and SAHA mediated reactivation from latency on day 12. Shown is n=1. ARVs: antiretrovirals: 100 nM Efavirenz, 200 nM Lamivudine and 200 nM Raltegravir. C. TT-44881 and TT-44863 inhibit Tat-Rev fusion protein transactivation. SIIB: a reporter HEK293T stable cell line where the essential apical region of TAR was replaced by the 29-nucleotide stem-loop IIB (SIIB) subdomain of the HIV-1 RRE. A Tat-Rev fusion protein activates transcription of luciferase when Rev binds SIIB, bringing Tat and P-TEFb to RNAPII at the HIV promoter. Shown is mean±SEM of n=4.

FIG. 5. TT-44881, TT-44863 and TT-44856 reduce Tat steady state levels. A. Lead hits reduce overexpressed Flag-Tat stability in HEK293T cells without affecting overexpressed GFP stability. Triptolide: 1 nM, TT-44856 and TT-44863: 40 μM, TT-44881: 60 μM. Shown is representative of n=3. B. Lead hits do not impact Tat or GFP mRNA expression in HEK293T cells. Shown is mean±SD of n=3. C. Impact of the lead hits on J-Lat A2 cells upon 17 hours stimulation with TNF-α (20 ng/mL). EPL: 10 μM, Triptolide (TRP): 1 nM, TT-44856 and TT-44863: 40 μM, TT-44881: 60 μM. Shown is representative of n=4. D. Lead hits reduce recombinant Tat stability in HEK293T cells. Triptolide: 1 nM, TT-44856 and TT-44863: 40 μM, TT-44881: 60 μM. Shown is n=1.

FIG. 6. TT-44881, TT-44863 and TT-44856 reduce Tat steady state levels via the proteasomal degradation pathways. A. Lead hits do not engage the lysosomal degradation pathway. Chloroquine (100 μM) or NH4Cl (50 mM) treatment (8 hrs) do not prevent lead hits induced Flag-Tat degradation. TT-44856 and TT-44863: 40 μM, TT-44881: 60 μM. Shown is n=1. B. Lead hits engage the proteasomal degradation pathways. MG132 treatment (17 hrs, 20 μM) prevents lead hits induced Flag-Tat degradation.TT-44856 and TT-44863: 40 μM, TT-44881: 60 μM, Triptolide: 1 nM. Shown below the densitometry data for the above western blots. Shown is representative of n=1-2.

FIG. 7. TT-44881, TT-44863 and TT-44856 reduce Tat steady state levels via the 26 proteasomal degradation pathway. A. Molecular docking of TT-44881 to the Tat PDB entry 1K5K and Cereblon protein. B/C. Lenalidomide treatment (Lena., 4 hrs, 40 μM) prevents Tat compounds induced Flag-Tat degradation. Triptolide (TRP): 1 nM, TT-44881: 60 μM, TT-44863: 40 μM, TT-44856: 40 μM, TT-46766: 40 μM (inactive analog of TT-44863), GB-04:60 μM (an inactive analog of TT-44881). Shown in C the densitometry data for the B. Shown is representative of n=1-2. D. VHL knockdown prevents TT-44881 induced Flag-Tat degradation. siCtl=siRNA control, siVHL=siRNA control. TT-44881: 60 μM. E. Knockdown of Cereblon (CRBN) diminishes TT-44856 induced Flag-Tat degradation. CD8 shRNA was used as a control. EPL: 10 μM, Triptolide (TRP): 1 nM, TT-44856: 40 μM. Shown is n=1.

FIG. 8. TT-44881, TT-44863 and TT-44856 's anti-HIV activity require the residue Y26A of Tat protein. A. Molecular docking to Tat PDB entry 1K5K. B. Lead hits do not reduce Tat steady levels when the cysteine and core domains are deleted. TT-44856 and TT-44863: 40 μM, TT-44881: 60 μM. Shown is representative of n=3. C. Lead hits do not reduce Tat steady levels when the residue Y26 is mutated. TT-44856 and TT-44863: 40 μM, TT-44881: 60 μM. Shown is representative of n=3. D. Lysine residue present in Tat 86 aa sequence. E. Mutation of K28R, K29R and K41R do not prevent lead hits induced Tat degradation. TT-44856 and TT-44863: 40 μM, TT-44881: 60 μM. Shown is n=1.

FIG. 9. TT-44881 and TT-44856 's anti-HIV activity engages different lysine residues of Tat protein. A. Additional mutations of the lysine residues to K28R, K29R and K41R disturb lead hits induced Tat degradation. TT-44856: 40 μM, TT-44881: 60 μM. Shown is representative of n=2. B. Mutations of a single lysine residue of the Tat protein does not impact the lead hits induced Tat degradation. TT-44856: 40 μM, TT-44881: 60 μM. Shown is n=1. C. The combined mutations of the most affected lysine residues from A (K19/71 for TT-44881, and K50, 51, 71, 85 for TT-44856) do not disturb lead hits induced Tat degradation. Shown is n=1.

FIG. 10. TT-44881's anti-HIV activity engages heterogenous lysine linked Ubiquitin chains (K48 and K33 chains). A. Example of covalent binding of the Ubiquitin chains to the lysine residues of the Tat protein. B. Schematic of the different Ubiquitin chains that can be linked to the Tat protein to induce its degradation or stabilization. C. The mutation of the residues K48 and K33 to alanine residues preventing both Ubiquitin chain linkage formation blocks TT-44881 induced Tat degradation. HEK293T cells overexpressing HA-Ubiquitin proteins (which have each lysine singly mutated to arginine or only one lysine present) and Flag-Tat protein were treated with TT-44881. Western blot assays were performed 24 hours later. TT-44881: 60 μM. Shown is representative of n=3.

FIG. 11 shows Synthesis of 3 hits.

### DEFINITIONS

Unless otherwise stated, structures depicted herein are also meant to include compounds that differ only in the presence of one or more isotopically enriched atoms. For example, compounds having the present structures except for the replacement of hydrogen by deuterium or tritium, or the replacement of ¹²C with ¹³C or ¹⁴C are within the scope of the disclosure. Such compounds are useful, for example, a imaging agents or as analytical tools or probes in biological assays.

As used herein, the term “salt” refers to any and all salts, and encompasses pharmaceutically acceptable salts.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₅ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, —Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₅ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

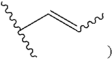

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and lor 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and lor 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14, electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl. In certain embodiments, the heteroaryl group is thiophene, benzothiophene, furan, isobenzofuran, pyrrole, imidazole, pyrazole, pyrazine, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, triazole, tetrazole, oxazole, isoxazole, thiazole, oxazole, or the like.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, SO₂OR^{aa}, —OSO₂R^{aa}, S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{aa})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NRC(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁s alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₅ perhaloalkyl, C₂₋₆ alkenyl, C₂₋₆ alkynyl, heteroC₁₋₆alkyl, heteroC₂-6alkenyl, heteroC₂₋₆alkynyl, C₃₋₁₀ carbocyclyl, C₆₋₁₀ aryl, 3-10 membered heterocyclyl, 5-10 membered heteroaryl; or two geminal R⁹⁹ substituents can be joined to form ═O or ═S; wherein X⁻ is a counterion.

The term “halo” or “halogen” refers to fluorine (fluoro, —F), chlorine (chloro, —C₁), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{b}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{a}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R¹¹)₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{aa}, —SO₂OR^{aa}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as a “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{cc})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb}) 2)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, a “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6^{th} ed. (501-502). Exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein).

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The term “heteroatom” refers to an atom that is not hydrogen or carbon. In certain embodiments, the heteroatom is nitrogen. In certain embodiments, the heteroatom is oxygen. In certain embodiments, the heteroatom is sulfur.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions, such as halide, hydroxide, carboxylate, sulfate, phosphate, nitrate, lower alkyl sulfonate, and aryl sulfonate.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for binding to, inhibiting, and/or promoting degradation of the human immunodeficiency virus type 1 (i.e., HIV-1) Tat protein. In certain embodiments, a therapeutically effective amount is an amount sufficient for treating a disease (e.g., a viral disease (e.g., HIV)).

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for binding to, inhibiting, and/or promoting degradation of the human immunodeficiency virus type 1 (i.e., HIV-1) Tat protein. In certain embodiments, a prophylactically effective amount is an amount sufficient for preventing a disease (e.g., a viral disease (e.g., HIV)).

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

### DETAILED DESCRIPTION OF CERTAIN EMBODIMENTS

Provided herein are compounds that bind to, inhibit, and/or promote degradation of the human immunodeficiency virus type 1 (i.e., HIV-1) Tat protein. In one aspect, the disclosure provides compounds of Formula (I) and pharmaceutically acceptable salts, solvates, hydrates, polymorphs, co-crystals, stereoisomers, isotopically labeled compounds, prodrugs, and pharmaceutical compositions thereof as described herein. In another aspect, the disclosure provides compounds of Formula (II) and pharmaceutically acceptable salts, solvates, hydrates, polymorphs, co-crystals, stereoisomers, isotopically labeled compounds, prodrugs, and pharmaceutical compositions thereof as described herein. In another aspect, the disclosure provides compounds of Formula (III) and pharmaceutically acceptable salts, solvates, hydrates, polymorphs, co-crystals, stereoisomers, isotopically labeled compounds, prodrugs, and pharmaceutical compositions thereof as described herein. The compounds as described herein may preferentially bind to the Tat protein at residue Y26 and be useful for the treatment of a disease (e.g., a viral disease (e.g., HIV)).

In one aspect, provided herein is a compound of Formula (I):

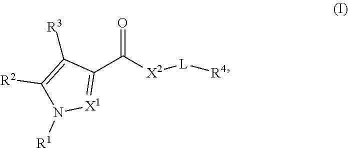

or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R² and R³ are independently hydrogen, halogen, substituted or unsubstituted carbocyclyl, substituted or unsubstituted heterocyclyl, substituted or unsubstituted aryl, or substituted or unsubstituted heteroaryl, or R² and R³, together with the atoms to which they are attached, are joined together to form a carbocyclic, heterocyclic, aryl, or heteroaryl ring, each of which may be optionally substituted;
L is a bond, substituted or unsubstituted alkylene, or substituted or unsubstituted heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In certain embodiments, the compound of Formula (I) is of Formula (I-b):

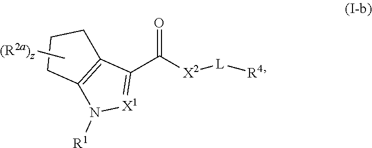

or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
L is a bond, substituted or unsubstituted alkylene, or substituted or unsubstituted heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{2a} is independently hydrogen, halogen, oxo, substituted or unsubstituted carbocyclyl, substituted or unsubstituted heterocyclyl, substituted or unsubstituted aryl, or substituted or unsubstituted heteroaryl; and
z is 1, 2, 3, 4, 5, or 6.

In certain embodiments, the compound of Formula (I) is of Formula (I-c):

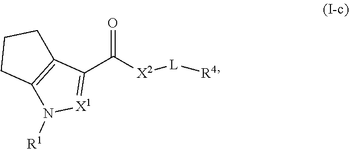

or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
L is a bond, substituted or unsubstituted alkylene, or substituted or unsubstituted heteroalkylene;
R⁴ is substituted or unsubstituted carbocyclyl, substituted or unsubstituted heterocyclyl, aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In certain embodiments, the compound of Formula (I) is of Formula (I-d):

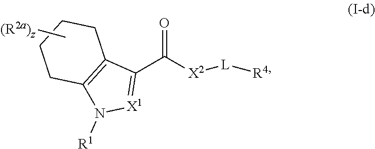

or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R² and R³ are independently hydrogen, halogen, carbocyclyl, heterocyclyl, aryl, or heteroaryl, or R² and R³, together with the atoms to which they are attached, are joined together to form a carbocyclic, heterocyclic, aryl, or heteroaryl ring;
L is substituted or unsubstituted alkylene or heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{2a} is hydrogen, halogen, oxo, hydroxy, optionally unsubstituted alkoxy, substituted or unsubstituted carbocyclyl, substituted or unsubstituted heterocyclyl, substituted or unsubstituted aryl, or substituted or unsubstituted heteroaryl; and
z is 1, 2, 3, 4, 5, or 6.

In certain embodiments, the compound of Formula (I) is of Formula (I-e):

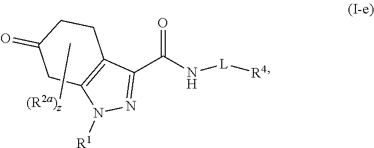

or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R² and R³ are independently hydrogen, halogen, substituted or unsubstituted carbocyclyl, substituted or unsubstituted heterocyclyl, substituted or unsubstituted aryl, or substituted or unsubstituted heteroaryl, or R² and R³, together with the atoms to which they are attached, are joined together to form a carbocyclic, heterocyclic, aryl, or heteroaryl ring, each of which may be optionally substituted;
L is a bond, substituted or unsubstituted alkylene, or substituted or unsubstituted heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{2a} is hydrogen, halogen, oxo, substituted or unsubstituted carbocyclyl, substituted or unsubstituted heterocyclyl, substituted or unsubstituted aryl, or substituted or unsubstituted heteroaryl; and
z is 1, 2, 3, 4, 5, or 6.

In certain embodiments, the compound of Formula (I) is of Formula (I-f):

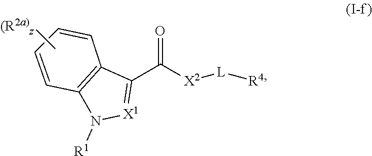

or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R² and R³ are independently hydrogen, halogen, substituted or unsubstituted carbocyclyl, substituted or unsubstituted heterocyclyl, substituted or unsubstituted aryl, or substituted or unsubstituted heteroaryl, or R² and R³, together with the atoms to which they are attached, are joined together to form a carbocyclic, heterocyclic, aryl, or heteroaryl ring, each of which may be optionally substituted;
L is substituted or unsubstituted alkylene, or substituted or unsubstituted heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{2a} is hydrogen, halogen, oxo, substituted or unsubstituted carbocyclyl, substituted or unsubstituted heterocyclyl, substituted or unsubstituted aryl, or substituted or unsubstituted heteroaryl; and
z is 1, 2, 3, 4, 5, or 6.

In certain embodiments, the compound of Formula (I) is of Formula (I-g):

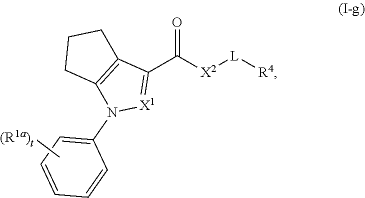

or a pharmaceutically acceptable salt, solvate, hydrate, polymorph, co-crystal, tautomer, stereoisomer, isotopically labeled compound, or prodrug thereof, wherein:
L is substituted or unsubstituted alkylene, or substituted or unsubstituted heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
R^{1a} is hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; and
t is 1, 2, 3, 4, or 5.

In certain embodiments, the compound of Formula (I) is of Formula (I-h):

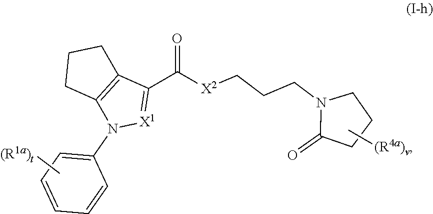

or a pharmaceutically acceptable salt, solvate, hydrate, polymorph, co-crystal, tautomer, stereoisomer, isotopically labeled compound, or prodrug thereof, wherein:
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{1a} is independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{4a} is independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl;
v is 1, 2, 3, 4, 5, or 6; and
t is 1, 2, 3, 4, or 5.

In certain embodiments, the compound of Formula (I) is of Formula (I-i):

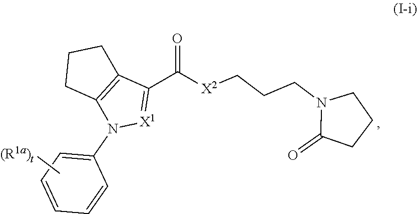

or a pharmaceutically acceptable salt, solvate, hydrate, polymorph, co-crystal, tautomer, stereoisomer, isotopically labeled compound, or prodrug thereof, wherein:
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{1a} is independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; and
t is 1, 2, 3, 4, or 5.

In one aspect, provided herein is a compound of Formula (I′):

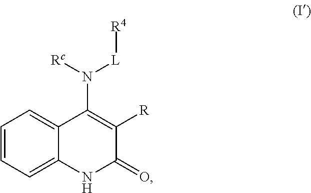

or a pharmaceutically acceptable salt, solvate, hydrate, polymorph, co-crystal, tautomer, stereoisomer, isotopically labeled compound, or prodrug thereof, wherein:
R is —CN, —C(═O)R^{b}, or —C(═O)N(R^{b})₂;
L is a bond, substituted or unsubstituted alkylene, or substituted or unsubstituted heteroalkylene;
R⁴ is substituted or unsubstituted carbocyclyl, substituted or unsubstituted heterocyclyl, substituted or unsubstituted aryl, or substituted or unsubstituted heteroaryl; and
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

Certain embodiments of the compounds of Formula (I) contain the substituent R¹. In certain embodiments, R¹ is hydrogen. In certain embodiments, R¹ is substituted or unsubstituted alkyl. In certain embodiments, R¹ is substituted or unsubstituted alkenyl. In certain embodiments, R¹ is substituted or unsubstituted alkynyl. In certain embodiments, R¹ is substituted or unsubstituted carbocyclyl. In certain embodiments, R¹ is substituted or unsubstituted heterocyclyl. In certain embodiments, R¹ is substituted or unsubstituted aryl, or substituted or unsubstituted heteroaryl. In certain embodiments, R¹ is substituted or unsubstituted aryl (e.g., phenyl). In certain embodiments, R¹ is substituted or unsubstituted heteroaryl.

Certain embodiments of the compounds of Formula (I) as described herein contains the substituent R². In certain embodiments, R² is hydrogen. In certain embodiments, R² is halogen. In certain embodiments, R² is substituted or unsubstituted carbocyclyl. In certain embodiments, R² is substituted or unsubstituted heterocyclyl. In certain embodiments, R² is substituted or unsubstituted aryl. In certain embodiments, R² is or substituted or unsubstituted heteroaryl.

Certain embodiments of the compounds of Formula (I) as described herein contains the substituent R³. In certain embodiments, R³ is hydrogen. In certain embodiments, R³ is halogen. In certain embodiments, R³ is substituted or unsubstituted carbocyclyl. In certain embodiments, R³ is substituted or unsubstituted heterocyclyl. In certain embodiments, R³ is substituted or unsubstituted aryl. In certain embodiments, R³ is or substituted or unsubstituted heteroaryl.

In certain embodiments, R² and R³, together with the atoms to which they are attached, are joined together to form a carbocyclic, heterocyclic, aryl, or heteroaryl ring, each of which may be optionally substituted.

In certain embodiments, R² and R³, together with the atoms to which they are attached, are joined together to form a substituted or unsubstituted carbocyclic ring (e.g., cyclopentyl, cyclohexyl, or cyclohexanone ring). In certain embodiments, R² and R³, together with the atoms to which they are attached, are joined together to form a substituted or unsubstituted heterocyclic ring. In certain embodiments, R² and R³, together with the atoms to which they are attached, are joined together to form a substituted or unsubstituted aryl (e.g., phenyl) ring. In certain embodiments, R² and R³, together with the atoms to which they are attached, are joined together to form a substituted or unsubstituted heteroaryl ring.

Certain embodiments of the compounds of Formula (I) as described herein contains the substituent R⁴. In certain embodiments, R⁴ is substituted or unsubstituted carbocyclyl. In certain embodiments, R⁴ is substituted or unsubstituted heterocyclyl. In certain embodiments, R⁴ is substituted or unsubstituted aryl. In certain embodiments, R⁴ is substituted or unsubstituted heteroaryl.

Certain embodiments of the compounds of Formula (I) as described herein contains the substituent X¹. In certain embodiments, X¹ is —N═. In certain embodiments, X¹ is —C(R^{a})═, wherein R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl. In certain embodiments, R^{a} is hydrogen, In certain embodiments, R^{a} is substituted or unsubstituted alkyl. In certain embodiments, R^{a} is substituted or unsubstituted carbocyclyl. In certain embodiments, R^{a} is substituted or unsubstituted heterocyclyl. In certain embodiments, R^{a} is substituted or unsubstituted aryl. In certain embodiments, R^{a} is substituted or unsubstituted heteroaryl.

Certain embodiments of the compounds of Formula (I) as described herein contains the substituent X². In certain embodiments, X² is —N(R^{c})—, wherein R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl. In certain embodiments, R^{c} is hydrogen. In certain embodiments, R^{c} is substituted or unsubstituted alkyl. In certain embodiments, R^{c} is substituted or unsubstituted carbocyclyl. In certain embodiments, R^{c} is substituted or unsubstituted heterocyclyl. In certain embodiments, R^{c} is substituted or unsubstituted aryl. In certain embodiments, R^{c} is substituted or unsubstituted heteroaryl. In certain embodiments, X² is —C(R^{b})₂—, wherein each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl. In certain embodiments, at least one instance of R^{b} is hydrogen. In certain embodiments, at least one instance of R^{b} is substituted or unsubstituted alkyl. In certain embodiments, at least one instance of R^{b} is substituted or unsubstituted carbocyclyl. In certain embodiments, at least one instance of R^{b} is substituted or unsubstituted heterocyclyl. In certain embodiments, at least one instance of R^{b} is substituted or unsubstituted aryl. In certain embodiments, at least one instance of R^{b} is substituted or unsubstituted heteroaryl. In certain embodiments, both instance of R^{b} are hydrogen.

Certain embodiments of the compounds of Formula (I) as described herein contains the substituent L. In certain embodiments, L is a bond. In certain embodiments, L is substituted or unsubstituted alkylene. In certain embodiments, L is substituted or unsubstituted C₁-C₆ alkylene. In certain embodiments, L is substituted or unsubstituted propylene. In certain embodiments, L is unsubstituted propylene. In certain embodiments, L is substituted or unsubstituted heteroalkylene.

Certain embodiments of the compounds of Formula (I) as described herein contain t instances of the substituent R^{1a}, wherein each instance of R^{1a} is independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl. In certain embodiments, at least one instance of R^{1a} is hydrogen. In certain embodiments, at least one instance of R^{1a} is halogen. In certain embodiments, at least one instance of R^{1a} is substituted or unsubstituted alkyl. In certain embodiments, at least one instance of R^{1a} is substituted or unsubstituted carbocyclyl. In certain embodiments, at least one instance of R^{1a} is substituted or unsubstituted heterocyclyl. In certain embodiments, at least one instance of R^{1a} is substituted or unsubstituted aryl. In certain embodiments, at least one instance of R^{1a} is substituted or unsubstituted heteroaryl. In certain embodiments, t is 1. In certain embodiments, t is 2. In certain embodiments, t is 3. In certain embodiments, t is 4. In certain embodiments, t is 5. In certain embodiments, t is 6.

Certain embodiments of the compounds of Formula (I) as described herein contain z instances of the substituent R^{2a}, wherein each instance of R^{2a} is hydrogen, halogen, oxo, substituted or unsubstituted carbocyclyl, substituted or unsubstituted heterocyclyl, substituted or unsubstituted aryl, or substituted or unsubstituted heteroaryl. In certain embodiments, at least one instance of R^{2a} is hydrogen. In certain embodiments, at least one instance of R^{2a} is halogen. In certain embodiments, at least one instance of R^{2a} is oxo. In certain embodiments, at least one instance of R^{2a} is substituted or unsubstituted carbocyclyl. In certain embodiments, at least one instance of R^{2a} is substituted or unsubstituted heterocyclyl. In certain embodiments, at least one instance of R^{2a} is substituted or unsubstituted aryl. In certain embodiments, at least one instance of R^{2a} is substituted or unsubstituted heteroaryl. In certain embodiments, z is 1. In certain embodiments, z is 2. In certain embodiments, z is 3. In certain embodiments, z is 4. In certain embodiments, z is 5. In certain embodiments, z is 6.

Certain embodiments of the compounds of Formula (I) as described herein contain v instances of the substituent R^{4a}, wherein each instance of R^{4a} is independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl. In certain embodiments, at least one instance of R^{4a} is hydrogen. In certain embodiments, at least one instance of R^{4a} is halogen. In certain embodiments, at least one instance of R^{4a} is substituted or unsubstituted alkyl. In certain embodiments, at least one instance of R^{4a} is substituted or unsubstituted carbocyclyl. In certain embodiments, at least one instance of R^{4a} is substituted or unsubstituted heterocyclyl. In certain embodiments, at least one instance of R^{4a} is substituted or unsubstituted aryl. In certain embodiments, at least one instance of R^{4a} is substituted or unsubstituted heteroaryl. In certain embodiments, v is 1. In certain embodiments, v is 2. In certain embodiments, v is 3. In certain embodiments, v is 4. In certain embodiments, v is 5.

In certain embodiments, the compound of Formula (I), or a pharmaceutically acceptable salt, solvate, hydrate, polymorph, co-crystal, tautomer, stereoisomer, isotopically labeled compound, or prodrug thereof, is a compound in Table 1.

In another aspect, provided herein is a compound of Formula (II′):

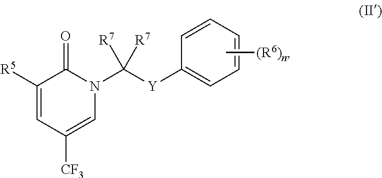

or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, hydroxy, optionally substituted alkoxy, substituted or unsubstituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, carbocyclyl, substituted or unsubstituted heterocyclyl, substituted or unsubstituted aryl, or substituted or unsubstituted heteroaryl;
Y is arylene, heteroarylene, —S(O₂)N(R^{d})—, —N(R^{d})C(═O)— or —C(═O)N(R^{d})—;
R^{d} is hydrogen, or substituted or unsubstituted alkyl; and
n is 1, 2, 3, 4, or 5.

In another aspect, provided herein is a compound of Formula (II):

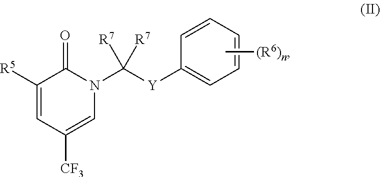

or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, hydroxy, optionally substituted alkoxy, substituted or unsubstituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, carbocyclyl, substituted or unsubstituted heterocyclyl, substituted or unsubstituted aryl, or substituted or unsubstituted heteroaryl;
Y is arylene, heteroarylene, —N(R^{d})C(═O)— or —C(═O)N(R^{d})—;
R^{d} is hydrogen, or substituted or unsubstituted alkyl; and
n is 1, 2, 3, 4, or 5.

In certain embodiments, the compound of Formula (II) is of Formula (II-a):

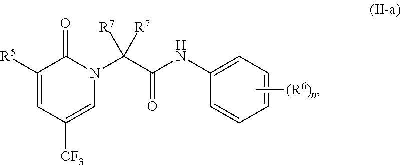

or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, optionally substituted alkoxy, substituted or unsubstituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
Y is substituted or unsubstituted arylene, substituted or unsubstituted heteroarylene, —N(R^{d})C(═O)— or —C(═O)N(R^{d})—; and
n is 1, 2, 3, 4, or 5.

In certain embodiments, the compound of Formula (II) is of Formula (II-b):

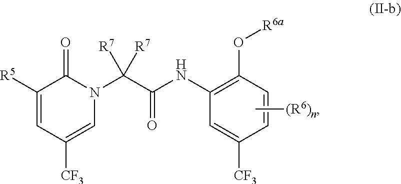

or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, optionally substituted alkoxy, substituted or unsubstituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R^{6a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
Y is substituted or unsubstituted arylene, substituted or unsubstituted heteroarylene, —N(R^{d})C(═O)— or —C(═O)N(R^{d})—; and
n is 1, 2, or 3.

In certain embodiments, the compound of Formula (II) is of Formula (II-c):

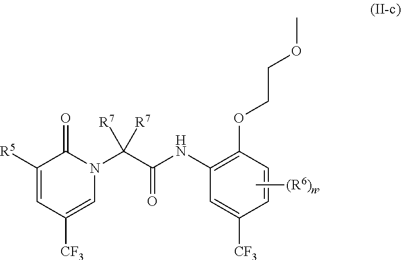

or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, a optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and
n is 1, 2, or 3.

In certain embodiments, the compound of Formula (II) is of Formula (II-d):

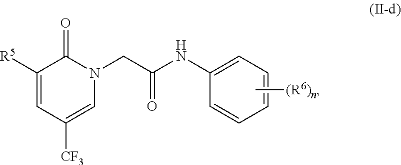

or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, hydroxy, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and
n is 1, 2, 3, 4, or 5.

In certain embodiments, the compound of Formula (II) is of Formula (II-e):

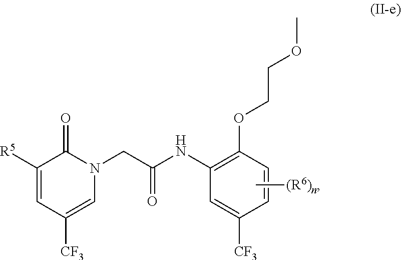

or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and
n is 1, 2, or 3.

In certain embodiments, the compound of Formula (II) is of Formula (II-f):

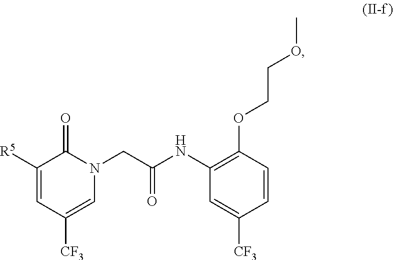

or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

Certain embodiments of the compounds of Formula (II) as described herein contains the substituent R⁵. In certain embodiments, R⁵ is hydrogen. In certain embodiments, R⁵ is halogen. In certain embodiments, R⁵ is chloro. In certain embodiments, R⁵ is bromo. In certain embodiments, R⁵ is substituted or unsubstituted alkyl. In certain embodiments, R⁵ is substituted or unsubstituted alkenyl. In certain embodiments, R⁵ is substituted or unsubstituted alkynyl. In certain embodiments, R⁵ is substituted or unsubstituted carbocyclyl. In certain embodiments, R⁵ is substituted or unsubstituted heterocyclyl. In certain embodiments, R⁵ is substituted or unsubstituted aryl. In certain embodiments, R⁵ is substituted or unsubstituted heteroaryl.

Certain embodiments of the compounds of Formula (II) as described herein contain n instances of the substituent R⁶. In certain embodiments, at least one instance of R⁶ is hydrogen. In certain embodiments, at least one instance of R⁶ is halogen. In certain embodiments, at least one instance of R⁶ is substituted or unsubstituted alkyl (e.g, heteroalkyl, haloalkyl). In certain embodiments, at least one instance of R⁶ is trifluoromethyl. In certain embodiments, at least one instance of R⁶ is hydroxy. In certain embodiments, at least one instance of R⁶ is optionally substituted alkoxy. In certain embodiments, at least one instance of R⁶ is substituted or unsubstituted acyl. In certain embodiments, at least one instance of R⁶ is substituted or unsubstituted alkenyl. In certain embodiments, at least one instance of R⁶ is substituted or unsubstituted alkynyl. In certain embodiments, at least one instance of R⁶ is substituted or unsubstituted carbocyclyl. In certain embodiments, at least one instance of R⁶ is substituted or unsubstituted heterocyclyl. In certain embodiments, at least one instance of R⁶ is substituted or unsubstituted aryl. In certain embodiments, at least one instance of R⁶ is substituted or unsubstituted heteroaryl. In certain embodiments, n is 1. In certain embodiments, n is 2. In certain embodiments, n is 3. In certain embodiments, n is 4. In certain embodiments, n is 5.

R^{6a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;

Certain embodiments of the compounds of Formula (II) as described herein contain n instances of the substituent R^{6a}. In certain embodiments, R^{6a} is hydrogen. In certain embodiments, R^{6a} is substituted or unsubstituted alkyl. In certain embodiments, R^{6a} is substituted or unsubstituted C₁-C₆ alkyl. In certain embodiments, R^{6a} is of the formula:

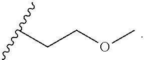 [SMILES: *CCOC]

In certain embodiments, R^{6a} is substituted or unsubstituted alkenyl. In certain embodiments, R^{6a} is substituted or unsubstituted alkynyl. In certain embodiments, R^{6a} is substituted or unsubstituted carbocyclyl. In certain embodiments, R^{6a} is substituted or unsubstituted heterocyclyl. In certain embodiments, R^{6a} is substituted or unsubstituted aryl. In certain embodiments, R^{6a} is substituted or unsubstituted heteroaryl.

Certain embodiments of the compounds of Formula (II) as described herein contains the substituent R⁷. In certain embodiments, at least in instance of R⁷ is hydrogen. In certain embodiments, at least in instance of R⁷ is substituted or unsubstituted alkyl. B14. In certain embodiments, at least in instance of R⁷ is substituted or unsubstituted C₁-C₆ alkyl.

In certain embodiments, at least in instance of R⁷ is methyl. In certain embodiments, at least in instance of R⁷ is optionally substituted alkoxy. In certain embodiments, at least in instance of R⁷ is substituted or unsubstituted alkenyl. In certain embodiments, at least in instance of R⁷ is substituted or unsubstituted alkynyl. In certain embodiments, at least in instance of R⁷ is carbocyclyl. In certain embodiments, at least in instance of R⁷ is substituted or unsubstituted heterocyclyl. In certain embodiments, at least in instance of R⁷ is substituted or unsubstituted aryl. In certain embodiments, at least in instance of R⁷ is substituted or unsubstituted heteroaryl.

Certain embodiments of the compounds of Formula (II) as described herein contains the substituent Y. In certain embodiments, Y is substituted or unsubstituted arylene. In certain embodiments, Y is substituted or unsubstituted heteroarylene. In certain embodiments, Y is —S(O₂)N(R^{d})—, wherein R^{d} is hydrogen, or substituted or unsubstituted alkyl. In certain embodiments, Y is —N(R^{d})C(═O)—, wherein R^{d} is hydrogen, or substituted or unsubstituted alkyl. In certain embodiments, Y is —C(═O)N(R^{d})—, wherein R^{d} is hydrogen, or substituted or unsubstituted alkyl. In certain embodiments, R^{d} is hydrogen. In certain embodiments, R^{d} is substituted or unsubstituted alkyl.

In certain embodiments, the compound of Formula (II), or a pharmaceutically acceptable salt, solvate, hydrate, polymorph, co-crystal, tautomer, stereoisomer, isotopically labeled compound, or prodrug thereof, is a compound in Table 2.

In one aspect, disclosed herein are compounds of Formula (III′):

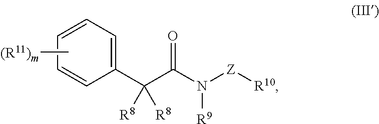

or a pharmaceutically acceptable salt, solvate, hydrate, polymorph, co-crystal, tautomer, stereoisomer, isotopically labeled compound, or prodrug thereof, wherein:
each R¹¹ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁸ is independently hydrogen, halogen, hydroxy, —N(R^{e})₂— substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
or one instance of R⁸ and R¹¹ is joined together with the atoms to which they are attached to form a 5 or 6 membered substituted or unsubstituted carbocyclic, substituted or unsubstituted heterocyclic, or substituted or unsubstituted heteroaryl ring;
each instance of R^{e} is independently hydrogen, substituted or unsubstituted alkyl, or substituted or unsubstituted acyl;
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
or one instance of R⁸ and R⁹ is joined together with the atoms to which they are attached to form a substituted or unsubstituted 5 membered heterocyclic ring;
R¹⁰ is substituted or unsubstituted carbocyclyl, substituted or unsubstituted heterocyclyl, substituted or unsubstituted aryl, or substituted or unsubstituted heteroaryl;
Z is substituted or unsubstituted alkylene, substituted or unsubstituted heteroalkylene, or substituted or unsubstituted heteroarylene; and
m is 1, 2, 3, 4, or 5.

In another aspect, provided herein is a compound of Formula (III):

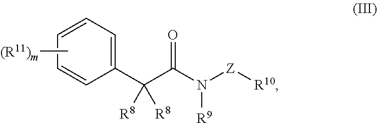

or a pharmaceutically acceptable salt, solvate, hydrate, polymorph, co-crystal, tautomer, stereoisomer, isotopically labeled compound, or prodrug thereof, wherein:
each R¹¹ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁸ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
R¹⁰ is substituted or unsubstituted carbocyclyl, substituted or unsubstituted heterocyclyl, substituted or unsubstituted aryl, or substituted or unsubstituted heteroaryl;
Z is substituted or unsubstituted alkylene, or substituted or unsubstituted heteroalkylene; and
m is 1, 2, 3, 4, or 5.

In certain embodiments, the compound of Formula (III) is of Formula (III-a):

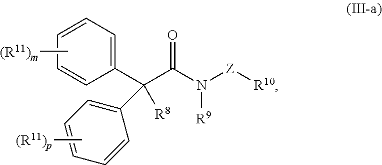

or a pharmaceutically acceptable salt, solvate, hydrate, polymorph, co-crystal, tautomer, stereoisomer, isotopically labeled compound, or prodrug thereof, wherein:
each R¹¹ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁸ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
R¹⁰ is carbocyclyl, heterocyclyl, aryl, or heteroaryl;
Z is substituted or unsubstituted alkylene or heteroalkylene;
p is 1, 2 3, 4, or 5; and
m is 1, 2, 3, 4, or 5.

In certain embodiments, the compound of Formula (III) is of Formula (III-b):

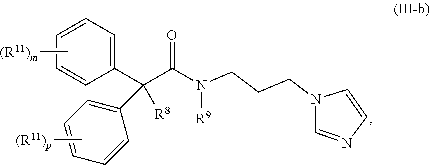

or a pharmaceutically acceptable salt, solvate, hydrate, polymorph, co-crystal, tautomer, stereoisomer, isotopically labeled compound, or prodrug thereof, wherein:
each R¹¹ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁸ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
m is 1, 2, 3, 4, or 5.

In certain embodiments, the compound of Formula (III) is of Formula (III-c):

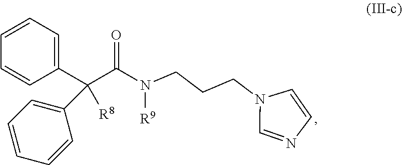

or a pharmaceutically acceptable salt, solvate, hydrate, polymorph, co-crystal, tautomer, stereoisomer, isotopically labeled compound, or prodrug thereof, wherein:
R⁸ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

Certain embodiments of the compounds of Formula (III) as described herein contains the substituent R⁸. In certain embodiments, at least one instance of R⁸ is hydrogen. In certain embodiments, at least one instance of R⁸ is halogen. In certain embodiments, at least one instance of R⁸ is substituted or unsubstituted alkyl. In certain embodiments, R⁸ is substituted or unsubstituted C₁-C₆ alkyl. In certain embodiments, R⁸ is methyl. In certain embodiments, at least one instance of R⁸ is substituted or unsubstituted alkenyl. In certain embodiments, at least one instance of R⁸ is substituted or unsubstituted alkynyl. In certain embodiments, at least one instance of R⁸ is substituted or unsubstituted carbocyclyl. In certain embodiments, at least one instance of R⁸ is substituted or unsubstituted heterocyclyl. In certain embodiments, at least one instance of R⁸ is substituted or unsubstituted aryl. In certain embodiments, at least one instance of R⁸ is substituted or unsubstituted phenyl. In certain embodiments, at least one instance of R⁸ is unsubstituted phenyl. In certain embodiments, at least one instance of R⁸ is substituted or unsubstituted heteroaryl

Certain embodiments of the compounds of Formula (III) as described herein contains the substituent R⁹. In certain embodiments, R⁹ is hydrogen. In certain embodiments, R⁹ is substituted or unsubstituted alkyl. In certain embodiments, R⁹ is substituted or unsubstituted carbocyclyl. In certain embodiments, R⁹ is substituted or unsubstituted heterocyclyl. In certain embodiments, R⁹ is substituted or unsubstituted aryl. In certain embodiments, R⁹ is substituted or unsubstituted heteroaryl.

Certain embodiments of the compounds of Formula (III) as described herein contains the substituent R¹⁰. In certain embodiments, R¹⁰ is substituted or unsubstituted carbocyclyl. In certain embodiments, R¹⁰ is substituted or unsubstituted heterocyclyl. In certain embodiments, R¹⁰ is substituted or unsubstituted aryl. In certain embodiments, R¹⁰ is substituted or unsubstituted heteroaryl. In certain embodiments, R¹⁰ is substituted or unsubstituted imidazolyl.

Certain embodiments of the compounds of Formula (III) as described herein contains m or p instances of the substituent R¹¹. In certain embodiments, at least one instance of R¹¹ is hydrogen. In certain embodiments, at least one instance of R¹¹ is halogen. In certain embodiments, at least one instance of R¹¹ is substituted or unsubstituted alkyl. In certain embodiments, at least one instance of R¹¹ is optionally substituted alkoxy. In certain embodiments, at least one instance of R¹¹ is optionally substituted acyl. In certain embodiments, at least one instance of R¹¹ is substituted or unsubstituted alkenyl. In certain embodiments, at least one instance of R¹¹ is substituted or unsubstituted alkynyl. In certain embodiments, at least one instance of R¹¹ is substituted or unsubstituted carbocyclyl. In certain embodiments, at least one instance of R¹¹ is substituted or unsubstituted heterocyclyl. In certain embodiments, at least one instance of R¹¹ is substituted or unsubstituted aryl. In certain embodiments, at least one instance of R¹¹ is substituted or unsubstituted heteroaryl.

In certain embodiments, m is 1. In certain embodiments, m is 2. In certain embodiments, m is 3. In certain embodiments, m is 4. In certain embodiments, m is 5. In certain embodiments, m is 6.

In certain embodiments, p is 1. In certain embodiments, p is 2. In certain embodiments, p is 3. In certain embodiments, p is 4. In certain embodiments, p is 5. In certain embodiments, p is 6.

Certain embodiments of the compounds of Formula (III) as described herein contains the substituent Z. In certain embodiments, Z is substituted or unsubstituted alkylene. In certain embodiments, Z is substituted or unsubstituted C₁-C₆ alkylene. In certain embodiments, Z is substituted or unsubstituted propylene. In certain embodiments, Z is substituted or unsubstituted heteroalkylene.

In certain embodiments,

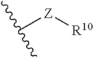

is of the formula:

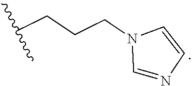 [SMILES: *CCCn1ccnc1]

In certain embodiments, the compound of Formula (III), or a pharmaceutically acceptable salt, solvate, hydrate, polymorph, co-crystal, tautomer, stereoisomer, isotopically labeled compound, or prodrug thereof, is a compound in Table 3.

In certain embodiments, the compound of the present invention, or a pharmaceutically acceptable salt, solvate, hydrate, polymorph, co-crystal, tautomer, stereoisomer, isotopically labeled compound, or prodrug thereof, is any compound disclosed herein. In certain embodiments, the compound of the present invention, or a pharmaceutically acceptable salt, solvate, hydrate, polymorph, co-crystal, tautomer, stereoisomer, isotopically labeled compound, or prodrug thereof, is any compound disclosed in Tables 1-3.

In certain embodiments, the compounds of any of the formulae herein (e.g., Formulae (I), (II), or (III)) as described herein bind HIV-1 Tat protein with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compounds of any of the formulae herein (e.g., Formulae (I), (II), or (III)) as described herein inhibit HIV-1 Tat protein with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compounds of any of the formulae herein (e.g., Formulae (I), (II), or (III)) as described herein promote degradation of HIV-1 Tat protein by at least about 10%, at least about 20%, at least about 30%, at least about 40%, at least about 50%, at least about 60%, at least about 70%, at least about 80%, at least about 90%, at least about 95%, at least about 98%, or at least about 99%.

### Pharmaceutical Compositions, Kits, and Administration

The present disclosure provides pharmaceutical compositions comprising a compound of any of the formulae herein (e.g., Formulae (I), (II), or (III), Tables 1-3), or a pharmaceutically acceptable salt, solvate, hydrate, polymorph, co-crystal, tautomer, stereoisomer, isotopically labeled compound, or prodrug thereof, and optionally a pharmaceutically acceptable excipient. In certain embodiments, the pharmaceutical composition described herein comprises a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, and a pharmaceutically acceptable excipient.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for binding HIV-1 Tat protein at least about 10%, at least about 20%, at least about 30%, at least about 40%, at least about 50%, at least about 60%, at least about 70%, at least about 80%, at least about 90%, at least about 95%, at least about 98%, or at least about 99%. In certain embodiments, the effective amount is an amount effective for binding HIV-1 Tat protein by a range between a percentage described in this paragraph and another percentage described in this paragraph, inclusive.

In certain embodiments, the effective amount is an amount effective for inhibiting HIV-1 Tat protein at least about 10%, at least about 20%, at least about 30%, at least about 40%, at least about 50%, at least about 60%, at least about 70%, at least about 80%, at least about 90%, at least about 95%, at least about 98%, or at least about 99%. In certain embodiments, the effective amount is an amount effective for inhibiting HIV-1 Tat protein by a range between a percentage described in this paragraph and another percentage described in this paragraph, inclusive.

In certain embodiments, the effective amount is an amount effective for promoting degradation of HIV-1 Tat protein by at least about 10%, at least about 20%, at least about 30%, at least about 40%, at least about 50%, at least about 60%, at least about 70%, at least about 80%, at least about 90%, at least about 95%, at least about 98%, or at least about 99%. In certain embodiments, the effective amount is an amount effective for promoting degradation of HIV-1 Tat protein by a range between a percentage described in this paragraph and another percentage described in this paragraph, inclusive.

A compound of any of the formulae herein (e.g., Formulae (I), (II), or (III)) or a pharmaceutical composition thereof, as described herein, can be administered in combination with one or more additional pharmaceutical agents (e.g., therapeutically and/or prophylactically active agents). The compound or pharmaceutical compositions described herein can be administered in combination with additional pharmaceutical agents that improve their activity (e.g., activity (e.g., potency and/or efficacy) in treating a disease (e.g., a viral disease (e.g., HIV)) in a subject in need thereof, in preventing a disease (e.g., a viral disease (e.g., HIV)) in a subject in need thereof, and/or in reducing the risk to develop a disease (e.g., a viral disease (e.g., HIV)) in a subject in need thereof), improve bioavailability, improve their ability to cross the blood-brain barrier, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound of any of the formulae herein (e.g., Formulae (I), (II), or (III)) and an additional pharmaceutical agent exhibit a synergistic effect that is absent in a pharmaceutical composition including a compound of any of the formulae herein (e.g., Formulae (I), (II), or (III)) or the additional pharmaceutical agent, but not both.

A compound of any of the formulae herein (e.g., Formulae (I), (II), or (III)), or a pharmaceutical composition thereof can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules, such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., a viral disease (e.g., HIV)). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the additional pharmaceutical agent is an antiretroviral agent. In certain embodiments, the antiretroviral agent is a fusion inhibitor (e.g., enfuvirtide, maraviroc), a nucleoside/nucleotide reverse transcriptase inhibitor (e.g., lamivudine, abacavir, zidovudine, stavudine, didanosine, emtricitabine, and tenofovir), a non-nucleoside reverse transcriptase inhibitor (e.g., delavirdine, efavirenz, etravirine, nevirapine), a protease inhibitor (e.g., amprenavir, fosamprenavir, atazanavir, darunavir, indinavir, lopinavir, ritonavir, nelfinavir, saquinavir, tipranavir), an integrase inhibitor (e.g., raltegravir, cabotegravir), or a combination thereof. In certain embodiments, the antiretroviral agent is efavirenz, lamivudine, raltegravir, or a combination thereof.

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage, the pharmaceutical compositions of this invention can be administered to humans and other animals orally, parenterally, intracisternally, intraperitoneally, topically, bucally, or the like, depending on the disease or condition being treated.

In certain embodiments, a pharmaceutical composition comprising a compound of any of the formulae herein (e.g., Formulae (I), (II), or (III)) is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the pharmaceutical composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a compound of any of the formulae herein (e.g., Formulae (I), (II), or (III)) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients, such as cocoa butter and suppository waxes, coloring agents, coating agents, sweetening, flavoring, and perfuming agents, may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., a viral disease (e.g., HIV)) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, a kit described herein may include one or more additional pharmaceutical agents described herein as a separate pharmaceutical composition.

Another object of the present disclosure is the use of a compound as described herein in the manufacture of a medicament for use in the treatment of a disorder or disease described herein. Another object of the present disclosure is the use of a compound as described herein for use in the treatment of a disorder or disease described herein.

### Methods of Treatment and Prevention

In another aspect, the present disclosure provides methods of treating or preventing a disease in a subject in need thereof, comprising administering to the subject in need thereof a compound of any of the formulae herein (e.g., Formulae (I), (II), or (III), Tables 1-3), or a pharmaceutical composition thereof. In certain embodiments, the present disclosure provides methods of treating a disease in a subject in need thereof, comprising administering to the subject in need thereof a compound of any of the formulae herein (e.g., Formulae (I), (II), or (III)), or a pharmaceutical composition thereof. In certain embodiments, the present disclosure provides methods of preventing a disease in a subject in need thereof, comprising administering to the subject in need thereof a compound of any of the formulae herein (e.g., Formulae (I), (II), or (III)), or a pharmaceutical composition thereof.

In another aspect, the present disclosure provides a compound of any of the formulae herein (e.g., Formulae (I), (II), or (III)), or a pharmaceutical composition thereof for use in treating or preventing a disease in a subject in need thereof. In another aspect, the present disclosure provides a compound of any of the formulae herein (e.g., Formulae (I), (II), or (III)), or a pharmaceutical composition thereof for use in treating a disease in a subject in need thereof. In another aspect, the present disclosure provides a compound of any of the formulae herein (e.g., Formulae (I), (II), or (III)), or a pharmaceutical composition thereof for use in preventing a disease in a subject in need thereof.

In another aspect, the present disclosure provides a compound of any of the formulae herein (e.g., Formulae (I), (II), or (III)), or a pharmaceutical composition thereof for use in the manufacture of a medicament for treatment or prevention of a disease in a subject in need thereof. In another aspect, the present disclosure provides a compound of any of the formulae herein (e.g., Formulae (I), (II), or (III)), or a pharmaceutical composition thereof for use in the manufacture of a medicament for treatment of a disease in a subject in need thereof. In another aspect, the present disclosure provides a compound of any of the formulae herein (e.g., Formulae (I), (II), or (III)), or a pharmaceutical composition thereof for use in the manufacture of a medicament for prevention of a disease in a subject in need thereof.

In certain embodiments, the disease is associated with human immunodeficiency virus type 1 (HIV-1) Tat protein. In certain embodiments, the disease is a viral disease (e.g., HIV). In certain embodiments, the disease is HIV. In certain embodiments, the disease is HIV-1.

In certain embodiments, the method or compound for use further comprises administering to the subject in need thereof an additional pharmaceutical agent. In certain embodiments, the method or compound for use further comprises administering to the subject in need thereof an antiretroviral agent. In certain embodiments, the antiretroviral agent is a fusion inhibitor (e.g., enfuvirtide, maraviroc), a nucleoside/nucleotide reverse transcriptase inhibitor (e.g., lamivudine, abacavir, zidovudine, stavudine, didanosine, emtricitabine, and tenofovir), a non-nucleoside reverse transcriptase inhibitor (e.g., delavirdine, efavirenz, etravirine, nevirapine), a protease inhibitor (e.g., amprenavir, fosamprenavir, atazanavir, darunavir, indinavir, lopinavir, ritonavir, nelfinavir, saquinavir, tipranavir), an integrase inhibitor (e.g., raltegravir, cabotegravir), or a combination thereof. In certain embodiments, the antiretroviral agent is efavirenz, lamivudine, raltegravir, or a combination thereof.

### Methods of Binding, Inhibiting, and Promoting Degradation of HIV-1 Tat Protein

In another aspect, the present disclosure provides methods of binding HIV-1 Tat protein in a subject in need thereof or in a cell, tissue, or biological sample, comprising administering to the subject in need thereof or contacting the cell, tissue, or biological sample with an effective amount of a compound of any of the formulae herein (e.g., Formulae (I), (II), or (III), Tables 1-3), or a pharmaceutically acceptable salt, solvate, hydrate, polymorph, co-crystal, tautomer, stereoisomer, isotopically labeled compound, or prodrug thereof, or a pharmaceutical composition thereof. In certain embodiments, the present disclosure provides methods of binding HIV-1 Tat protein in a subject in need thereof, comprising administering to the subject in need thereof an effective amount of a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or a pharmaceutical composition thereof. In certain embodiments, the present disclosure provides methods of binding HIV-1 Tat protein in a cell, tissue, or biological sample, comprising contacting the cell, tissue, or biological sample with an effective amount of a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or a pharmaceutical composition thereof.

In another aspect, the present disclosure provides methods of inhibiting HIV-1 Tat protein in a subject in need thereof or in a cell, tissue, or biological sample, comprising administering to the subject in need thereof or contacting the cell, tissue, or biological sample with an effective amount of a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or a pharmaceutical composition thereof. In certain embodiments, the present disclosure provides methods of inhibiting HIV-1 Tat protein in a subject in need thereof, comprising administering to the subject in need thereof an effective amount of a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or a pharmaceutical composition thereof. In certain embodiments, the present disclosure provides methods of inhibiting HIV-1 Tat protein in a cell, tissue, or biological sample, comprising contacting the cell, tissue, or biological sample with an effective amount of a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or a pharmaceutical composition thereof.

In another aspect, the present disclosure provides methods of promoting degradation of HIV-1 Tat protein in a subject in need thereof or in a cell, tissue, or biological sample, comprising administering to the subject in need thereof or contacting the cell, tissue, or biological sample with an effective amount of a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or a pharmaceutical composition thereof. In certain embodiments, the present disclosure provides methods of promoting degradation of HIV-1 Tat protein in a subject in need thereof, comprising administering to the subject in need thereof an effective amount of a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or a pharmaceutical composition thereof. In certain embodiments, the present disclosure provides methods of promoting degradation of HIV-1 Tat protein in a cell, tissue, or biological sample, comprising contacting the cell, tissue, or biological sample with an effective amount of a compound of any of the formulae herein (e.g., Formulae (I), (II), or (III)), or a pharmaceutically acceptable salt, solvate, hydrate, polymorph, co-crystal, tautomer, stereoisomer, isotopically labeled compound, or prodrug thereof, or a pharmaceutical composition thereof.

In certain embodiments, the cell, tissue, or biological sample is in vivo. In certain embodiments, the cell, tissue, or biological sample is in vitro.

### EXAMPLES

### Identification of TT-44881, TT-44856 and TT-44863.

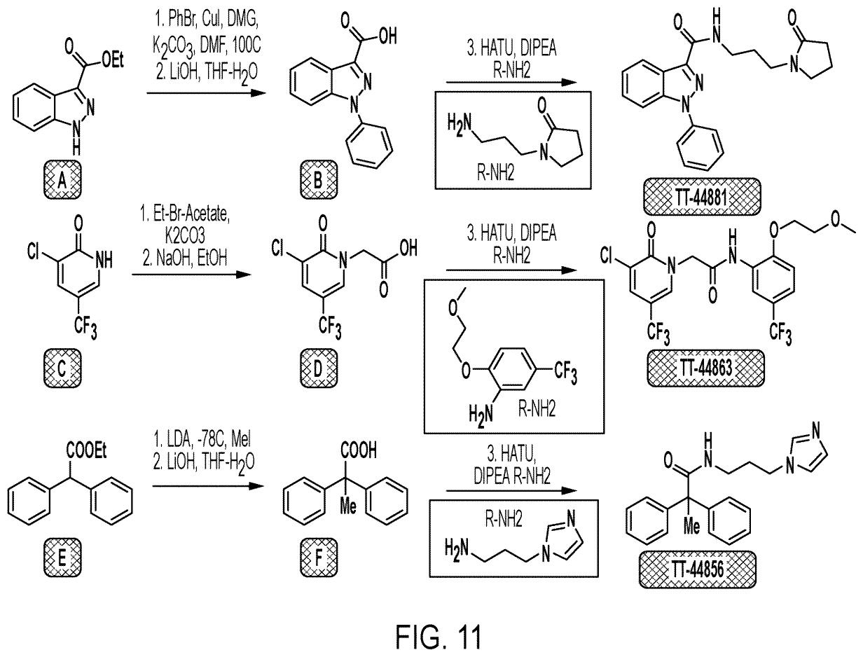

Compounds delineated herein can be made using standard chemical synthesis procedures and reagents known to one of ordinary skill in the art and as described herein. See, FIG. 11. The physicochemical properties and biological and functional activity of the compounds is assessed as described herein and using techniques known to one of ordinary skill in the art.

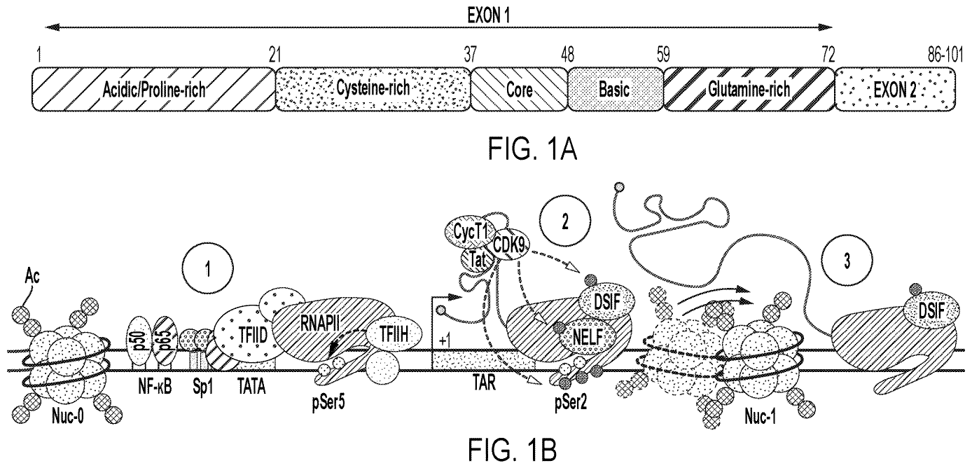

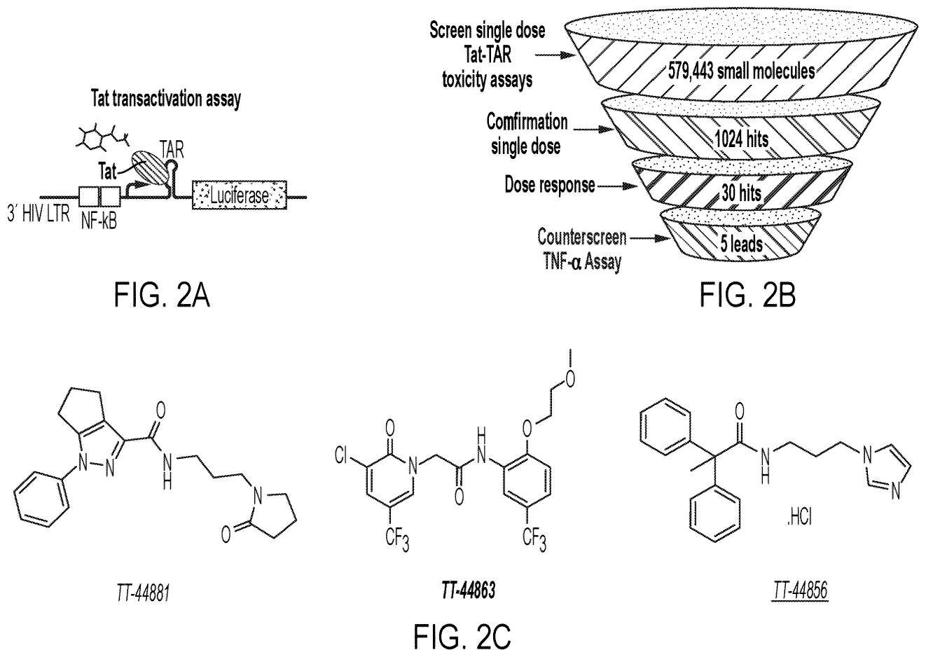

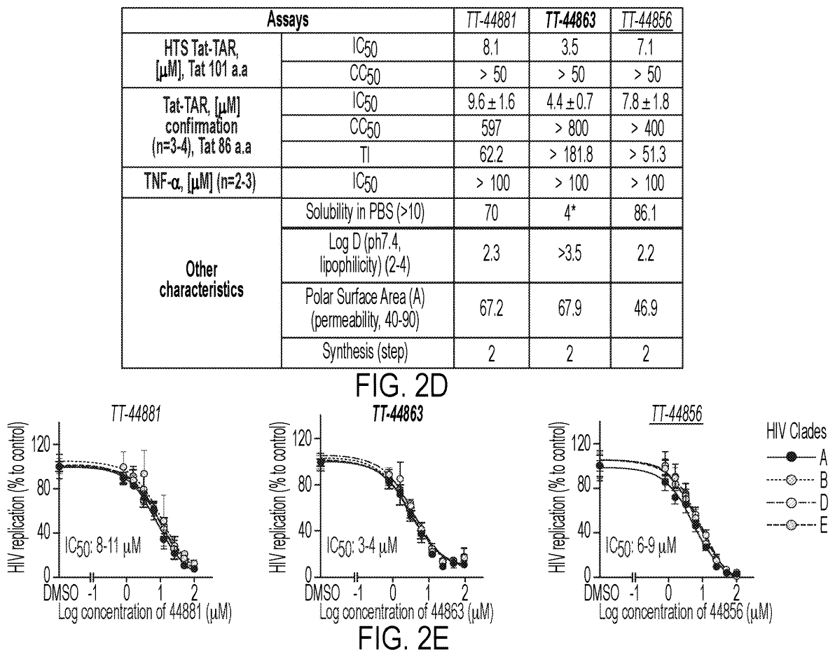

Tat is a 14 kDa protein that is synthesized from an mRNA joined from 2 coding exons (FIG. 1B). The first exon encodes amino acids 1-72 and (in most strains of HIV), the second exon encodes amino acids 73-101. An N-terminal 86-amino acid form of Tat produced in a few laboratory-passaged virus strains is frequently used to study Tat. The classic cell-based Tat transactivation assay of the HIV LTR driving luciferase was optimized as a reporter gene for HTS9 (FIG. 2A). The assay concept is exceptionally robust, includes all the cellular components needed for transcriptional activation, and is very well-suited for an HTS for Tat antagonists. Compared with alternative approaches, this classic assay consistently outperformed the others. Notably, a clone of HeLa-CD4 cells stably expressing HIV-1-LTR-Luciferase (termed “PL11”) with very low basal transcriptional activity was used, improving the dynamic range and sensitivity, markedly increasing the chances of identifying Tat-specific inhibitors. LTR activation is triggered by transfection of a plasmid expressing HIV-1HXB3 Tat (101 a.a.) driven by the phosphoglycerate (PGK) promoter. The assay called “Tat-TAR assay” was validated, and found robust and very well suited for an HTS platform, with a signal to background ratio of 407:1, a signal to noise ratio of 10:1, and a Z′=0.67. The cytotoxicity screen was performed in parallel (Cell-Titer-Glo, Promega), showing a signal to background ratio 898:1, signal to noise ratio 21:1 and a Z′=0.86. The HTS of the 579,443 compounds from multiple libraries (Chembridge, Life Chemicals Kinase Library, NIAID Enamine) resulted in the identification of 1024 hits (FIG. 2B). These hits were counter-screened in a Tat independent transactivation assay using TNFα to activate PL11 cells, resulting in the selection of 30 hits. Three of these compounds, TT-44863 (Pyridinones scaffold), TT-44881 (Pyrazole-3-Carboxamides), and TT-44856 (2,2-Diphenylacetamide scaffold) were found to be Tat specific and have reasonable scaffolds for optimization (FIG. 2C). Equivalent IC50 of these compounds against both Tat 86 and 101 residues was confirmed in the Tat-TAR assay (FIG. 2D), thus confirming the ability to use NL4.3 virus (Tat 86 a.a) in infectivity assays. Their activity was confirmed against Tat 86 a.a. from HIV clade B, C and E in the cell-based Tat-TAR assay (FIG. 2E) and selected for further investigation.

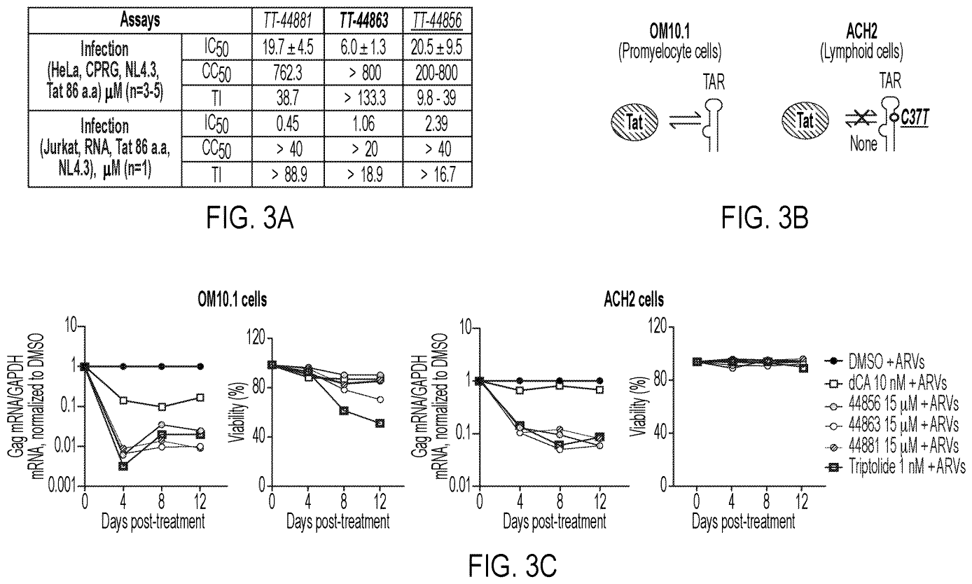

Next, the activity of these leads against HIV infection was evaluated using the reporter cell line HeLa-CD4-LTR-LacZ, which stably expresses the β-galactosidase (β-Gal) gene under the control of the HIV 5′ LTR. The HIV NL4.3 strain was used for these studies and β-Gal activity was measured 72 hrs post infection (FIG. 3A). Cytotoxicity assays using Cell-Titer-Glo were performed in parallel to assess cell viability (FIG. 3A). TT-44881, TT-44863 and TT-44856 displayed an IC50 of 19.7±4.5 μM, 6.0±1.3 μM and 20.5±9.5 μM respectively without signs of toxicity (FIG. 3A). Their anti-HIV activity was further assessed in acute replication of Jurkat T cells with the NL4.3 strain (FIG. 3A). The ability of these small molecules to inhibit HIV was also extended to latent cell models of infection such as OM-10.1 and ACH-2 cells (FIG. 3B-C). These OM10.1 cells contain a single integrated wild-type provirus, while ACH-2 cells contain a single integrated provirus with a TAR mutation C37T, defective Tat-TAR feedback loop (FIG. 3B). In conjunction with a cocktail of ARVs (Efavirenz 100 nM, Lamivudine 200 nM and Raltegravir 200 nM), treatment with the lead hits resulted in a ~10-to-100-fold decrease of cell-associated HIV Gag mRNA expression over the 12-day period treatment without observed toxicity (FIG. 3C). The non-specific Tat degrader Triptolide used as control reduced Gag mRNA in both cell lines, while dCA the positive control Tat-TAR inhibitor, only inhibited transcription in OM10.1 cells as expected. These results suggested the lead compounds have a mechanism of action more like Triptolide as opposed to dCA. In addition, results in clonal infected cells also indicated that these compounds were transcriptional inhibitors.

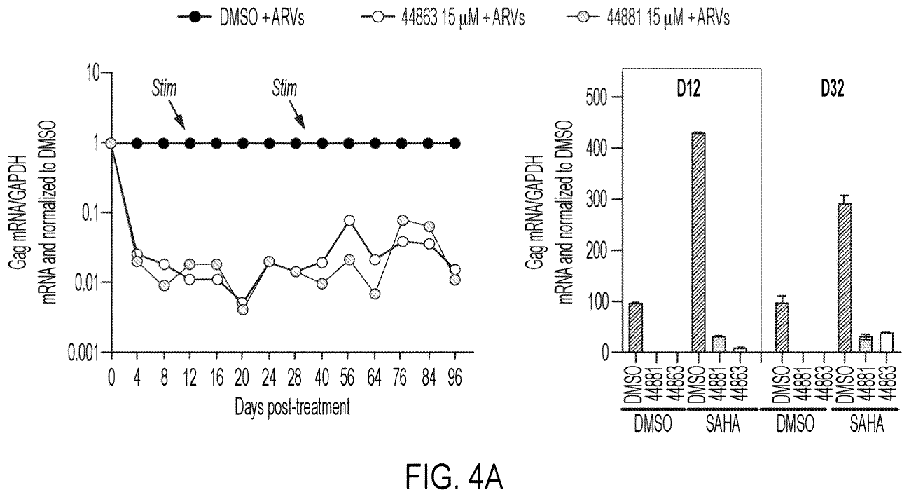

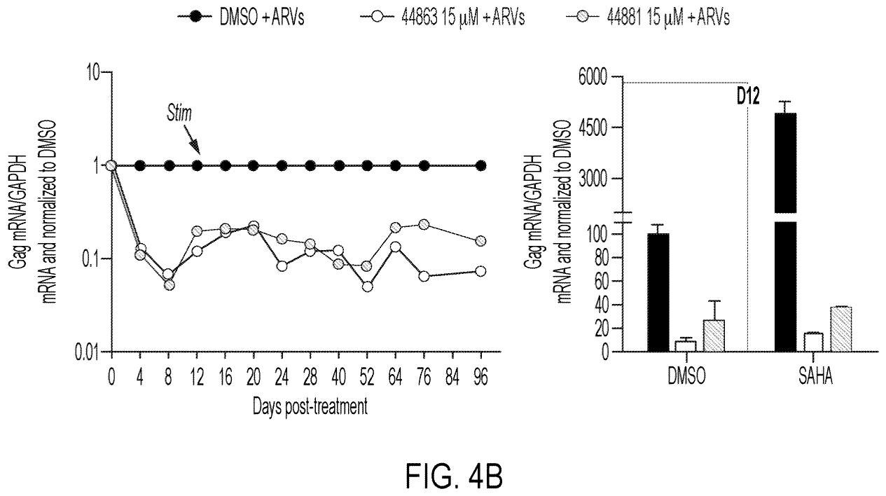

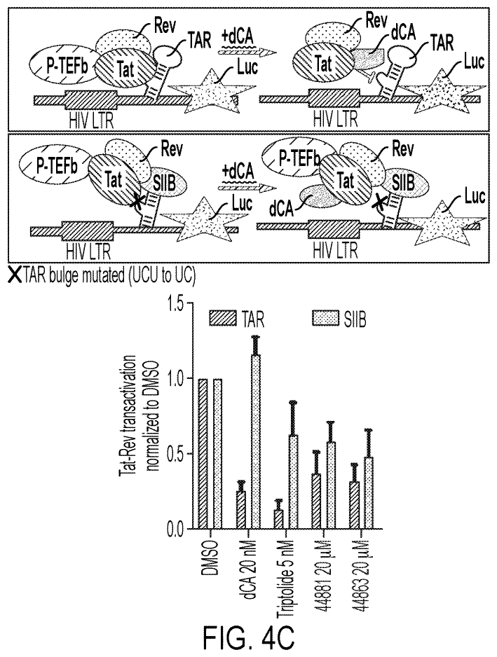

Based on prior work with dCA, it is hypothesized that long-term treatment with Tat inhibitors overtime favors a repressive chromatin environment at the HIV transcription locus that dampens viral reactivation upon stimulation with latency reversing agents (LRAs). As such, OM10.1 and ACH2 cells were treated with TT-44863 and TT-44881 in the presence of the cocktail of ARVs for a 96-day period, confirming their activity on both cell lines without affecting cell viability (FIG. 4A-B), and their ability to block viral reactivation was evaluated when cells were exposed to the histone deacetylase (HDAC) inhibitor SAHA at day 12 and 32. Both compounds potently blocked SAHA mediated reactivation from latency compared to DMSO+ARVs control, in both cell lines and at both time points (FIG. 4A-B). To further investigate the specificity of the three lead compounds on Tat-TAR interaction, Flag-Tat-Rev fusion protein transactivation of the LTR containing HIV-1 RRE was performed in apical region of TAR as previously described²² (FIG. 4C). dCA used as control interfered with the Flag-Tat-Rev to TAR binding, but not with the Flag-Tat-Rev transactivation of the TAR-SIIB promoter (FIG. 4C). All three leads along with Triptolide showed inhibition of both TAR and TAR-SIIB, suggesting an alternative mechanism of action to dCA (confirming the data obtained in the latent cell models (FIG. 3C, 4A-B)), likely involving Tat degradation.

### TT-44881, TT-44856 and TT-44863 Reduce Tat Steady State Levels.

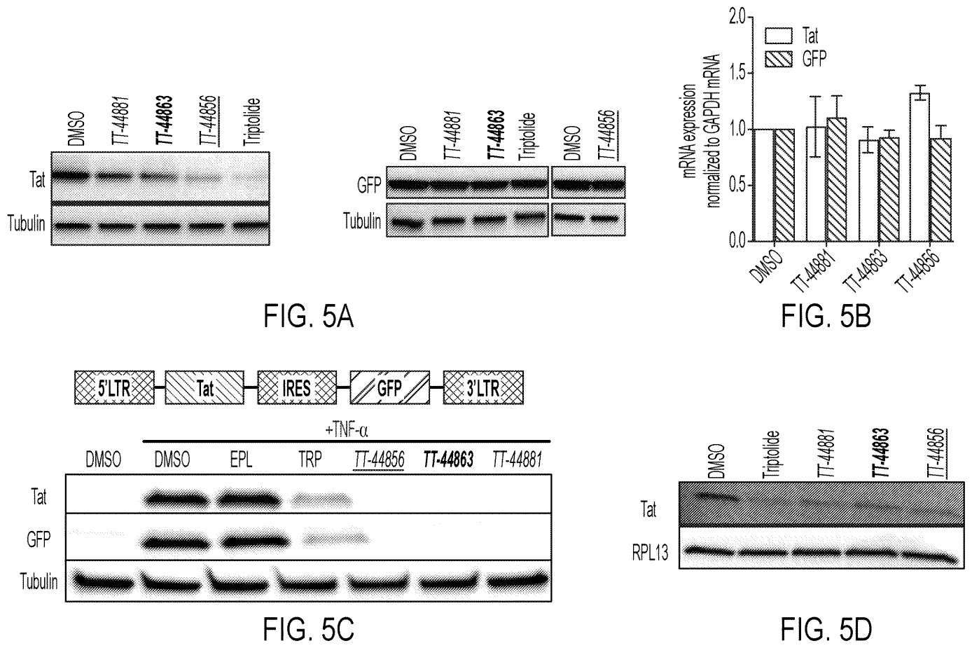

Next, the effect of the three leads on the alteration of Tat steady state levels was assessed. Tat expression from an integrated provirus, or from HIV acute infection is generally too low to be detected by western blot. Furthermore, antibodies against Tat are known for their lack of avidity. Tat degradation was thus assessed upon transfection. Flag-Tat or Flag-GFP were individually transfected into HEK293T and 24 hrs later, TT-44856, TT-44863 or TT-44881 were added to the culture, followed by Western blot 24 hrs later (dubbed “degradation assay”) (FIG. 5A). Tat protein degradation was observed for all 3 lead compounds without affecting Flag-Tat mRNA levels (FIG. 5B). In contrast, they did not affect GFP expression, used as control (FIG. 5A-B), and as expected Triptolide nonspecifically degraded Flag-Tat (FIG. 5A). These results were confirmed using J-Lat A2 cells, which contains the LTR-Tat-IRES-GFP construct and expresses Flag-Tat and GFP only upon stimulation with LRAs (FIG. 5C). These cells were treated with TNFα (previously determined not be degraded by the three leads in FIG. 2D) in the presence of TT-44856, TT-44863 or TT-44881, followed by western blot with an anti-Flag antibody 17 hrs later. Flag-Tat and GFP (driven by Tat) were strongly reduced by all three compounds (FIG. 5C). Similar results were obtained with the positive control Triptolide. Finally, the leads' impact on recombinant Tat protein in HEK293T cells was assessed (FIG. 5D). HEK293T cells were incubated with recombinant Tat for 4 hrs to allow Tat internalization, followed by several thorough washes to remove unincorporated Tat. Cells were then incubated with all three leads (or Triptolide) for an additional 2 hrs. Cells were lysed and analyzed by western blot with an anti-Tat serum. The lead hits and Triptolide degraded Tat as opposed to the DMSO control, indicating a post-transcriptional mechanism of action for all three leads.

### TT-44881, TT-44856 and TT-44863 Degrade Tat Via the 26S Proteasomal Degradation Pathway.

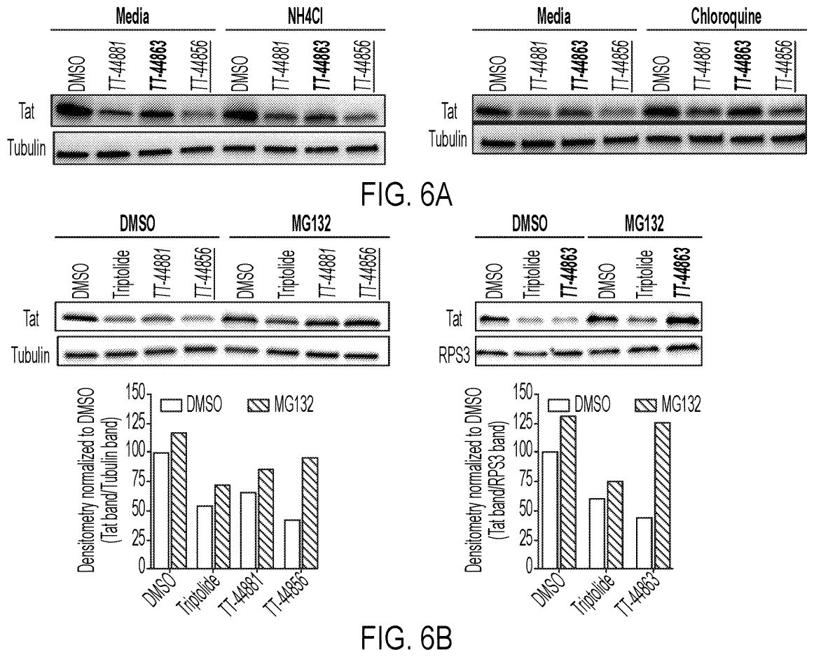

Degradation of Tat has been reported in the literature in multiple ways, including the lysosomal pathway²³, the ubiquitin-independent 20S proteasomal pathway (due to its unfolded structure)^{24,25}, and the ubiquitin-dependent 26S proteasomal pathway²⁶. It was first assessed whether Tat was targeted to the lysosomal pathway by the lead compounds. HEK293T cells were transfected with Flag-Tat for 24 hrs, incubated with the compounds for an additional 24 hrs, then treated for 8 hrs with either 100 μM chloroquine or 50 mM NH₄Cl, both known to inhibit lysosomal hydrolases by reducing acidification. Flag-Tat degradation was then revealed by western blot with an anti-Flag antibody (FIG. 6A). No relative changes in Flag-Tat degradation were observed with/without chloroquine and NH₄Cl between DMSO and leads, suggesting that the leads' mechanism of action does not engage the lysosomal degradation pathway. Both the 20S and the 26S proteasomal pathways were next investigated using MG132, an inhibitor of the catalytic 35 subunit of 20S present in both pathways²⁷. The degradation assay was carried out as above, but compounds were simultaneously incubated with MG132 20 μM or DMSO for 17 hrs followed by western blot. In the presence of MG132, Tat degradation was rescued, but the rescue was higher in the presence of the lead compounds (FIG. 6B). Similar results were observed with the Triptolide control28. Altogether, the data suggest that the lead molecules engage the proteasomal degradation pathway(s).

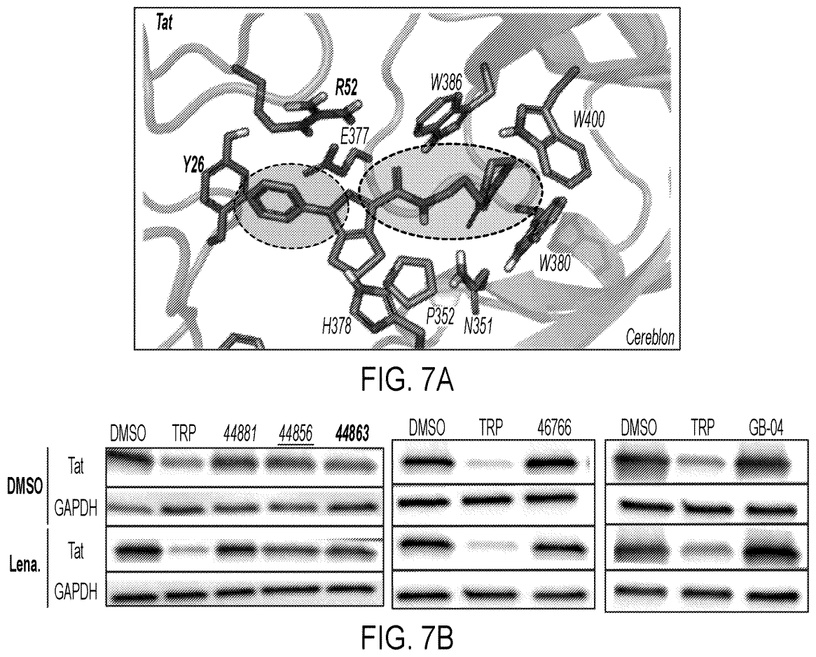

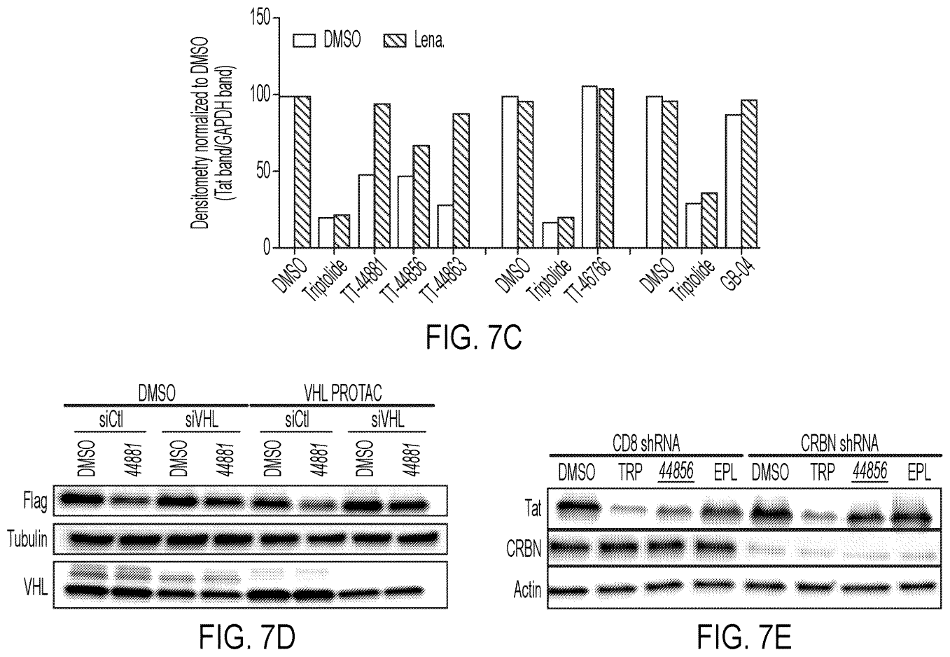

Protein ubiquitination occurs by sequential recruitment of ubiquitin-activating enzyme E1, ubiquitin-conjugating enzyme E2, and ubiquitin ligase E3 (using ubiquitin, substrate protein, and ATP). The selectivity in protein ubiquitination is conferred by E3 ubiquitin ligases that recognize the substrate proteins²⁹ and the human genome contains more than 600 E3 ubiquitin ligases²⁹. To determine whether the small molecule leads trigger the 20S or the 26S proteasomal pathways, the Tat degradation assay was performed in the presence of commercially available E3 ligase inhibitors, including Lenalidomide a E3 ubiquitin ligase Cereblon inhibitor. Cereblon has not been previously reported to ubiquitinate Tat. The degradation assays were performed as above with Lenalidomide (40 μM) or DMSO incubation for 4 hrs in the presence of the lead molecules (FIG. 7A). In the presence of leads, Lenalidomide prevented Flag-Tat degradation to a higher degree than with DMSO, suggesting the 26S proteasomal pathway is engaged upon hits treatment; and Cereblon is likely involved in Tat ubiquitination (FIG. 7A). As controls, an inactive analog of TT-44863 (TT-46766) and an inactive analog of TT-44881 (GB-04) were used, and both compounds did not affect Flag-Tat steady state (FIG. 7A). Triptolide used as a positive control did not engage the 26S proteasomal pathway, but only the 20S proteasomal pathway. To confirm the engagement of Cereblon in the mechanism of action of the lead compounds, shRNAs were used to stably knock down Cerebelon, or CD8 control, in HEK293T cells, and the degradation assay for TT-44856 was performed. The results indicated that in the absence of Cereblon, TT-44856 was no longer engaging Flag-Tat for degradation (FIG. 7B). These results strongly suggest that the lead small molecules engage Cereblon to ubiquitinate Tat followed by 26S proteasomal degradation, acting likely as molecular glues³⁰.

The involvement of the ubiquitous VHL E3 ligase in the activity of TT-44881 inducing Tat degradation was also investigated. HEK293T cells overexpressing Tat protein and VHL siRNAs (or si control) were treated with either TT-44881 (60 μM) and DMSO or TT-44881 with a VHL PROTAC (CM11, 500 nM) for 24 hours. siRNAs and PROTACs were used together to maximize the depletion of both VHL isoforms (30 and 19 kDa). Tat was no longer depleted by TT-44881 when both isoforms of VHL were depleted (FIG. 7E), suggesting the involvement of VHL E3 ligase in the mechanism of action of TT-44881.

NRON, a lncRNA expressed in HIV latently infected CD4⁺ T cells, has been previously reported to promote Tat degradation through the ubiquitin proteasomal pathway. NRON links Tat to components of ubiquitin proteasomal pathway (CUL4B and PSMD11) resulting in its ubiquitination and degradation³¹. The host U-box E3 ubiquitin ligase protein CHIP was also shown to promote Tat degradation via the same pathway³². Cereblon has not previously been reported to have Tat as a substrate. NRON, a lncRNA expressed in HIV latently infected CD4⁺ T cells, has been previously reported to promote Tat degradation through the ubiquitin proteasomal pathway. NRON links Tat to components of ubiquitin proteasomal pathway (CUL4B and PSMD11) resulting in its ubiquitination and degradation³¹. The host U-box E3 ubiquitin ligase protein CHIP was also shown to promote Tat degradation via the same pathway³². The role of these E3 ligases on the activity of the lead compounds can also be investigated. The degree of Tat ubiquitination in the presence of the molecules can also be studied in denaturing conditions. Finally, inhibitors of the other components of the 26S proteasomal pathways such as E1, E2, p97 can also be explored to fully uncover the step impacted by the leads.

### TT-44881, TT-44856 and TT-44863 Mediate Tat Degradation Via Tat Y26.

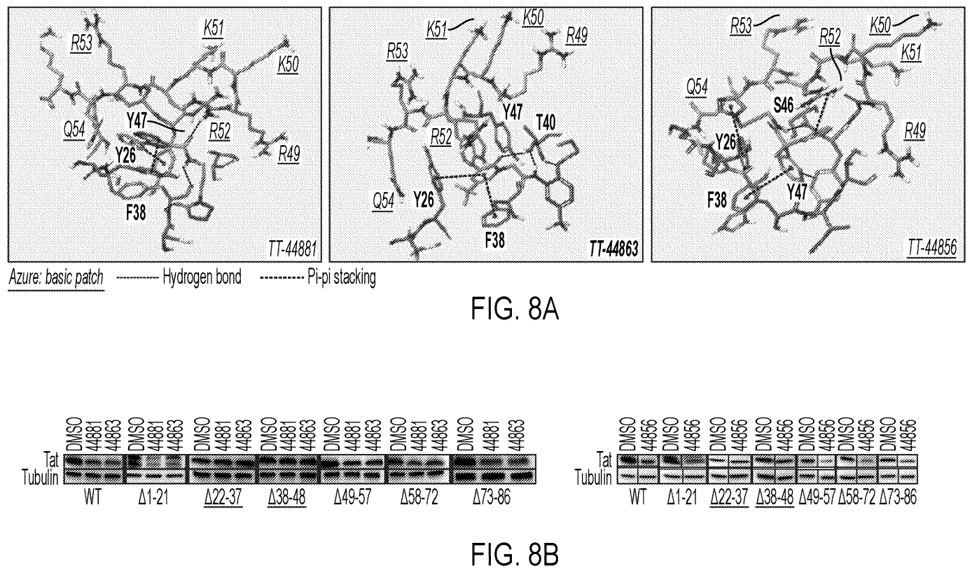

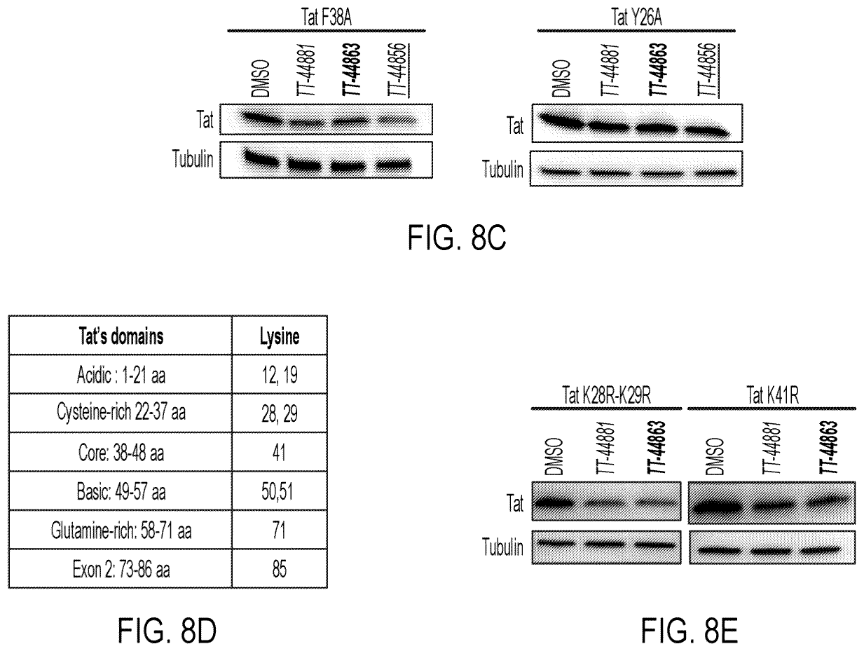

The molecular docking of HIV Tat protein bound to dCA was previously reported, informed by a series of more than 60 analogs of dCA, using AutoDock Vina with the NMR HIV Tat Mal protein (D clade) as a template (Protein data Bank, PDB entry 1K5K)₂₁. Docking of Tat with TT-44863, TT-44881 and TT-44856 showed a preferential binding to the Tat residues Y26 and F38 (FIG. 8A). Single changes to residues Y26 and F38 decrease Tat transactivation activity³³⁻³⁶. When TT-44863 and TT-44881 were docked to TAR (TAR/Acepromazine complex (PDB ID: 1LVJ)), no binding was identified, reinforcing their specificity to Tat protein. To validate the molecular docking model, Tat variants with either deletions or mutated residues were used in degradation assays in the presence of leads. All three molecules no longer degraded Flag-Tat when the cysteine or core regions were truncated (FIG. 8B). A Y26A mutation in Tat's cysteine region ablated lead hits' ability to mediate Tat degradation (FIG. 8C). On the other hand, the F38A mutation, only weakly reduced lead hits mediated degradation (FIG. 8C).

Tat 86 a.a. has 9 lysine residues (FIG. 8D) that could be engaged by lysine 48-linked ubiquitin chains or nonconventional ubiquitin chains, i.e., non-Lys-48 or non-Lys-63, to be degraded through the 26S proteasomal pathway³⁷³⁸. Point mutations were introduced in Tat residues K28-29R and K41R present in the core and cysteine regions, however these did not alter the activity of the leads in degradation assays (FIG. 8D-E). Tat lysine residues required for leads' degradation engagement can be determined.

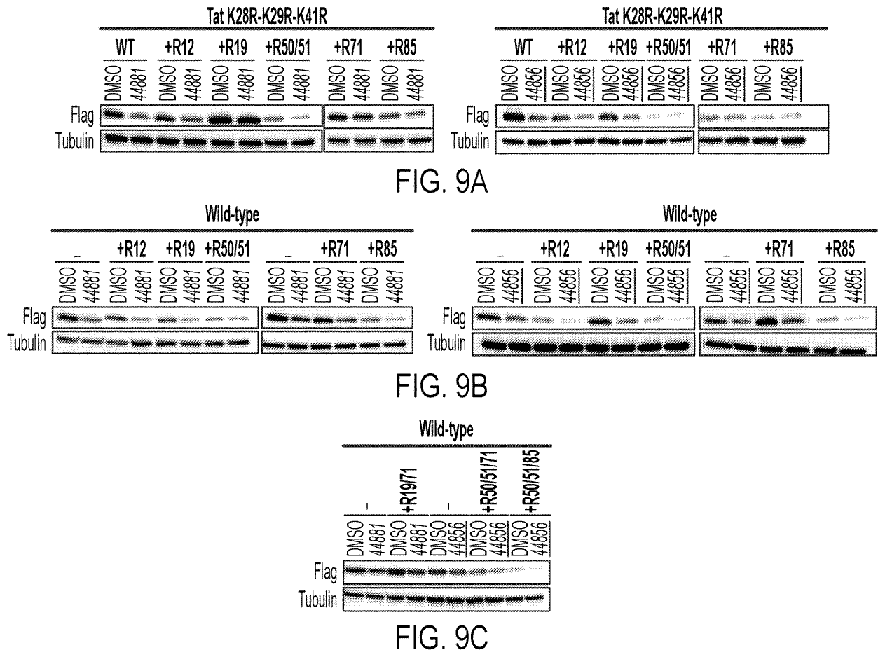

In addition to K28-29-K41R, other Tat's lysine residues were mutated and degradation assays performed in the presence of TT-44881 or TT-44856. The mutation of the K19 or K71 to arginine reduced TT-44881 induced Tat degradation (FIG. 9A), while the activity of TT-44856 required either K50/51, K71 or K85 (FIG. 9A). Single Tat lysine mutations or combined mutations were also performed as shown in FIG. 9A, in the presence of TT-44881 or TT-44856; however, all the mutants were similarly degraded to the control (FIG. 9B-C).

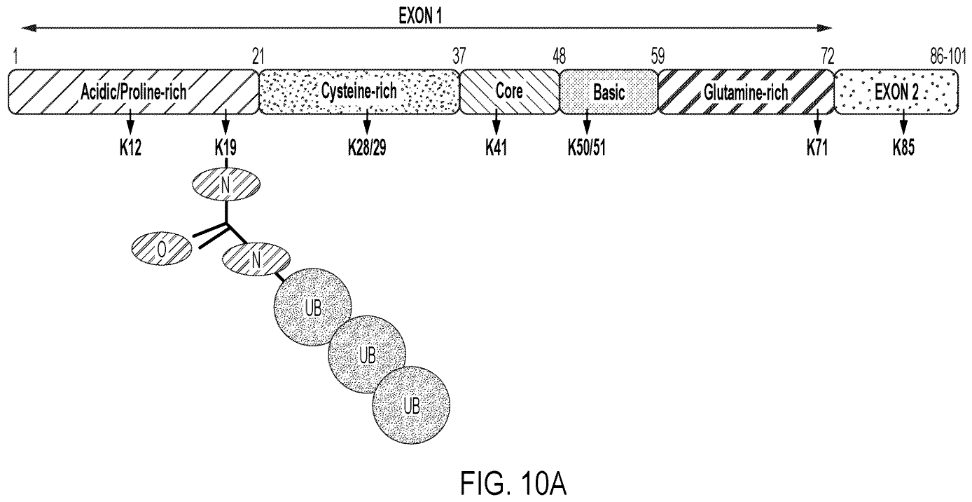

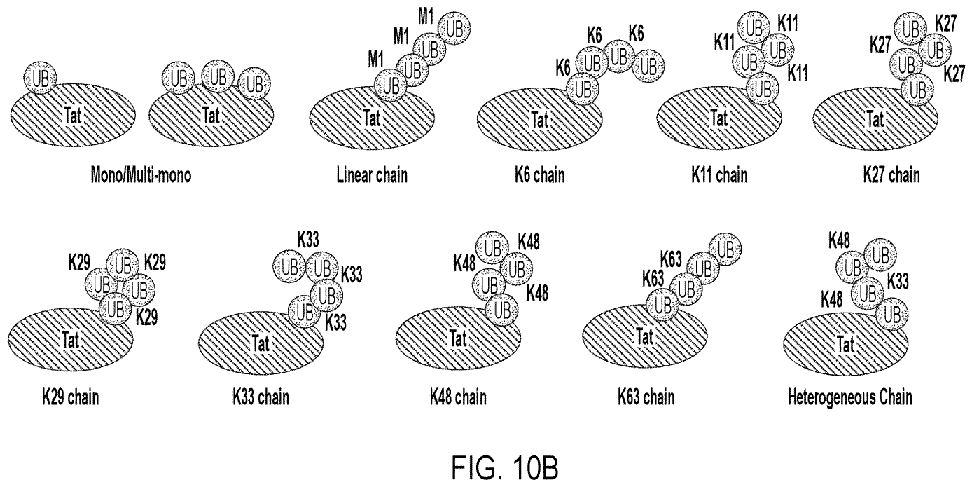

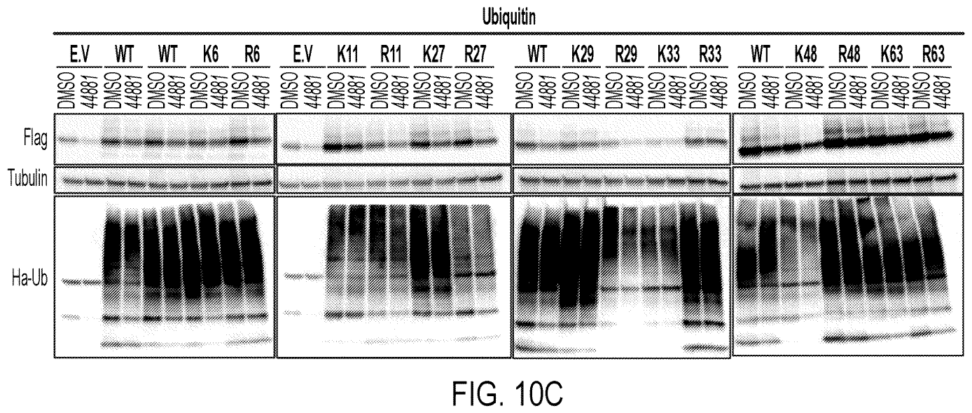

Which Ubiquitin linkage engaged in the TT-44881 degradation activity was also investigated. HEK293T cells were transfected with Tat protein and HA-Ubiquitin which has each lysine singly mutated to arginine or only one lysine present. It was confirmed that K48-linked ubiquitin chains are involved in TT-44881 activity, K33 linked ubiquitin chains also seemed to be engaged by the compound (FIG. 10), suggesting that TT-44881 may utilize heterogenous Ubiquitin chains.

### Generation and Optimization of Lead Compounds by Structure Activity Relationship Studies.

The three lead hits showed antiviral activity in the 6.0-20 μM range without cytotoxicity, a reasonable starting point to develop analogs with improved antiviral activity. The synthesis of all three hit analogs is considered very straight-forward (FIG. 11A), with no anticipated issues in scaling-up their production. Aza-indole ester A is arylated with bromobenzene and then saponified with lithium hydroxide to provide carboxylic acid intermediate B, which is converted to corresponding amide using HATU to give the desired TT-44881. Similarly, N-alkylation of pyridinone C with ethyl bromoacetate, followed by saponification and amide coupling with corresponding amine affords TT-44863. Synthesis of TT-44856 has been achieved by LDA mediated alkylation of diphenyl ester E followed by hydrolysis and amide formation using HATU and corresponding amine.

Derivatization at various sites on each scaffold allows for structural diversity and the development of novel small molecule inhibitors with improved activity and drug-like properties (FIG. 11B). The docking results for TT-44863 (described in FIG. 8A) implied that the trifluoromethyl and the methoxyethyl groups on the right-hand side of TT-44863 are amenable to modifications such that reducing the length of the ether linker in analog TT-46600 increased its potency by ~5 fold in HeLa-CD4 cell NL4.3 infection assays (FIG. 11C). However, reducing the length of the ether linker and replacing the trifluoromethyl on the right-hand side with C₁, decreased the potency of TT-44863 by ~5 fold (TT-46603), suggesting the importance of the CF₃ in the interaction with Tat (FIG. 11C). A series of approximately 60 analogs are being synthesized for each of the hits. For each scaffold, analogs include a change in ring sizes and aromaticity, as well as introduction of alkyls, cycloalkyls, aryls, heteroaryls or different ring systems bearing sulfonyl or sulfonamide substituents. Novel analogs will be prioritized and selected for in vivo PK studies in mice based on activity, potency and in vitro ADME [IC50<500 nM; CC50>30 μM; MLM t½>30 min; Sol.>10 μM]. Having scaffold diversity with good chemical tractability enables optimization of activity and drug-like properties for the identification of novel Tat inhibitors.

As shown in Tables 1-3 below, compounds of any of the formulae herein (e.g., Formulae (I), (II), (II′), (III′), or (III)), or pharmaceutically acceptable salts, solvates, hydrates, polymorphs, co-crystals, tautomers, stereoisomers, isotopically labeled compounds, or prodrugs thereof, bind, inhibit, and/or promote degradation of Tat protein.

TABLE 1
Tat protein inhibition for analogs of TT-44881 evaluated using the reporter cell line
HeLa-CD4-LTR-LacZ.

| | | Infectivity IC50 | Infectivity CC50 |
|---|---|---|---|
| Compound Structure | ID | (uM) | (uM) |
| 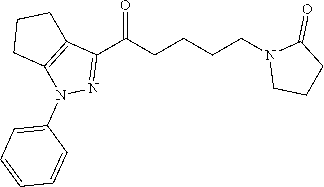 [SMILES: O=C(CCCCN1CCCC1=O)c1nn(-c2ccccc2)c2c1CCC2] | 44881 | 1-20 | >100 |
| 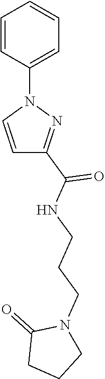 [SMILES: O=C(NCCCN1CCCC1=O)c1ccn(-c2ccccc2)n1] | 45732 | >100 | >100 |
| 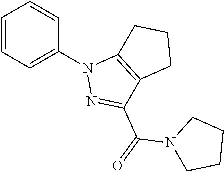 [SMILES: O=C(c1nn(-c2ccccc2)c2c1CCC2)N1CCCC1] | 46607 | >100 | >100 |
| 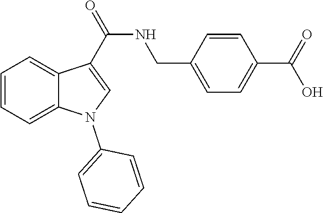 [SMILES: O=C(O)c1ccc(CNC(=O)c2cn(-c3ccccc3)c3ccccc23)cc1] | 41784 | >100 | >100 |
| 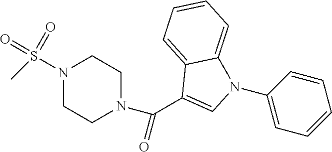 [SMILES: CS(=O)(=O)N1CCN(C(=O)c2cn(-c3ccccc3)c3ccccc23)CC1] | 41798 | >100 | >100 |
| 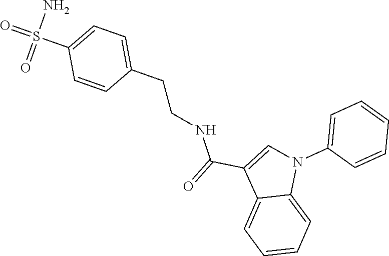 [SMILES: NS(=O)(=O)c1ccc(CCNC(=O)c2cn(-c3ccccc3)c3ccccc23)cc1] | 41799 | 5-20 | >100 |
| 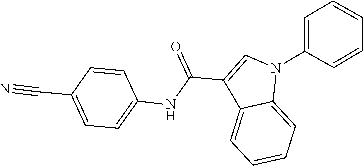 [SMILES: N#Cc1ccc(NC(=O)c2cn(-c3ccccc3)c3ccccc23)cc1] | 41905 | 1-10 | 1-10 |
| 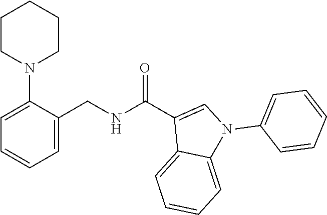 [SMILES: O=C(NCc1ccccc1N1CCCCC1)c1cn(-c2ccccc2)c2ccccc12] | 41924 | >100 | >100 |
| 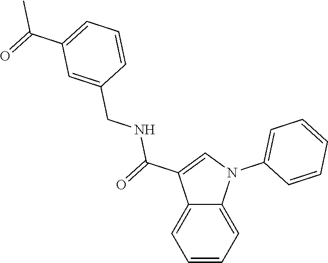 [SMILES: CC(=O)c1cccc(CNC(=O)c2cn(-c3ccccc3)c3ccccc23)c1] | 41967 | >100 | >100 |
| 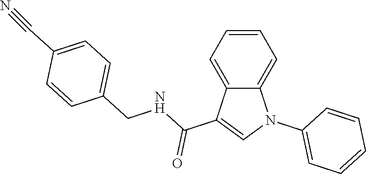 [SMILES: N#Cc1ccc(CNC(=O)c2cn(-c3ccccc3)c3ccccc23)cc1] | 41969 | 12.37 | >100 |
| 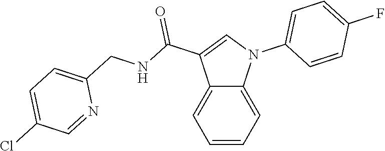 [SMILES: O=C(NCc1ccc(Cl)cn1)c1cn(-c2ccc(F)cc2)c2ccccc12] | 41980 | >100 | 35.65 |
| 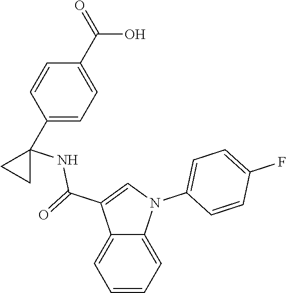 [SMILES: O=C(O)c1ccc(C2(NC(=O)c3cn(-c4ccc(F)cc4)c4ccccc34)CC2)cc1] | 42097 | >100 | > 100 |
| 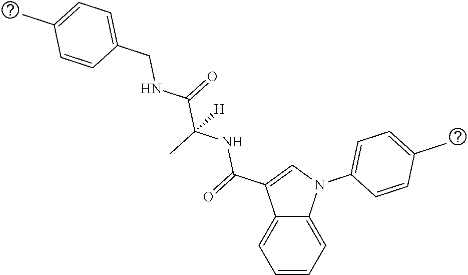 | 42174 | >100 | >100 |
| 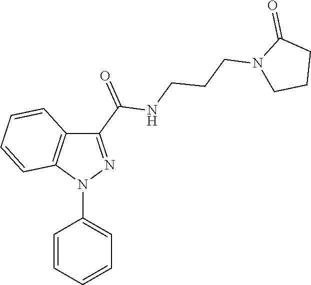 [SMILES: O=C(NCCCN1CCCC1=O)c1nn(-c2ccccc2)c2ccccc12] | 46715 | 50 - 100 | >100 |
| 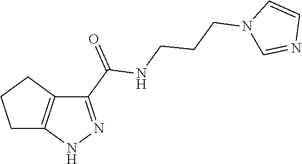 [SMILES: O=C(NCCCn1ccnc1)c1n[nH]c2c1CCC2] | 46726 | >100 | >100 |
| 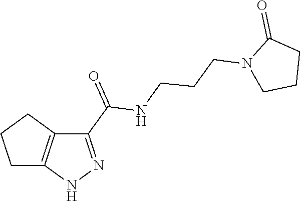 [SMILES: O=C(NCCCN1CCCC1=O)c1n[nH]c2c1CCC2] | 46727 | >100 | >100 |
| 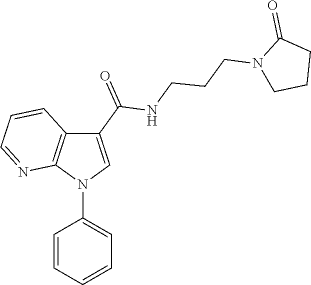 [SMILES: O=C(NCCCN1CCCC1=O)c1cn(-c2ccccc2)c2ncccc12] | 46751 | >100 | >100 |
| 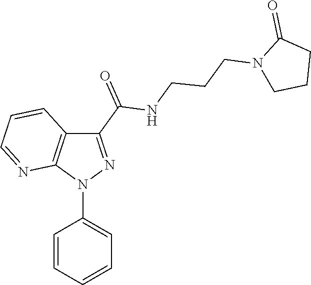 [SMILES: O=C(NCCCN1CCCC1=O)c1nn(-c2ccccc2)c2ncccc12] | 46752 | >100 | >100 |
| 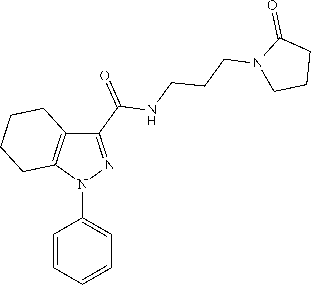 [SMILES: O=C(NCCCN1CCCC1=O)c1nn(-c2ccccc2)c2c1CCCC2] | 46819 | >50 | >50 |
| 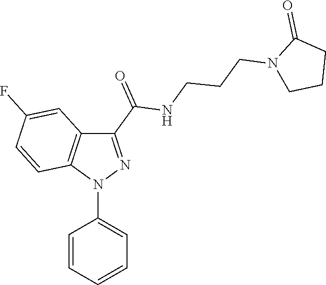 [SMILES: O=C(NCCCN1CCCC1=O)c1nn(-c2ccccc2)c2ccc(F)cc12] | 46820 | 50 - 100 | >100 |
| 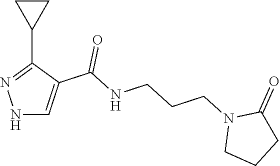 [SMILES: O=C(NCCCN1CCCC1=O)c1c[nH]nc1C1CC1] | 46821 | >100 | >100 |
| 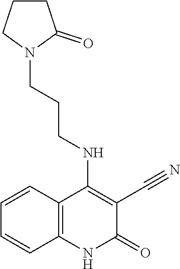 [SMILES: N#Cc1c(NCCCN2CCCC2=O)c2ccccc2[nH]c1=O] | 46822 | >100 | >100 |
| 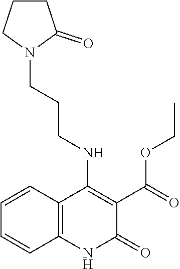 [SMILES: CCOC(=O)c1c(NCCCN2CCCC2=O)c2ccccc2[nH]c1=O] | 46823 | >100 | >100 |
| 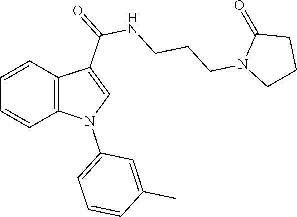 [SMILES: Cc1cccc(-n2cc(C(=O)NCCCN3CCCC3=O)c3ccccc32)c1] | 46824 | >100 | >100 |
| 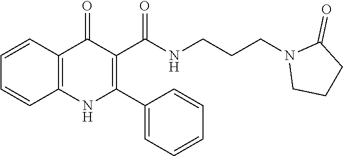 [SMILES: O=C(NCCCN1CCCC1=O)c1c(-c2ccccc2)[nH]c2ccccc2c1=O] | 46825 | >100 | >100 |
| 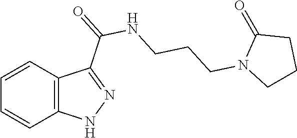 [SMILES: O=C(NCCCN1CCCC1=O)c1n[nH]c2ccccc12] | 46826 | >100 | >100 |
| 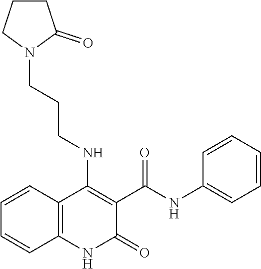 [SMILES: O=C(Nc1ccccc1)c1c(NCCCN2CCCC2=O)c2ccccc2[nH]c1=O] | 46827 | >100 | >100 |
| 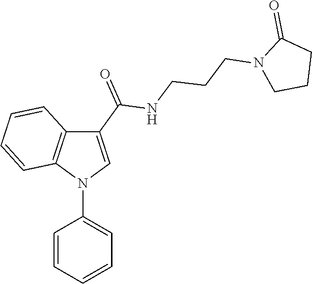 [SMILES: O=C(NCCCN1CCCC1=O)c1cn(-c2ccccc2)c2ccccc12] | 46878 | 50-100 | >100 |
| 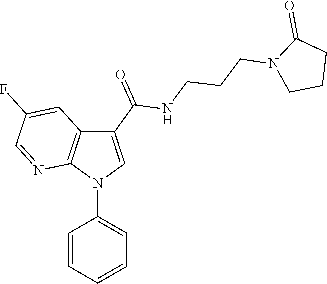 [SMILES: O=C(NCCCN1CCCC1=O)c1cn(-c2ccccc2)c2ncc(F)cc12] | 46879 | 1-50 | >100 |
| 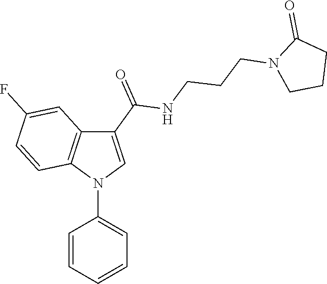 [SMILES: O=C(NCCCN1CCCC1=O)c1cn(-c2ccccc2)c2ccc(F)cc12] | 46880 | 50-100 | 50-100 |
| 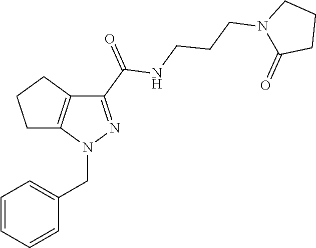 [SMILES: O=C(NCCCN1CCCC1=O)c1nn(Cc2ccccc2)c2c1CCC2] | 46885 | >100 | >100 |
| 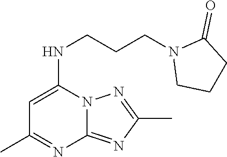 [SMILES: Cc1cc(NCCCN2CCCC2=O)n2nc(C)nc2n1] | 46930 | >100 | >100 |
| 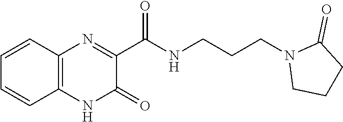 [SMILES: O=C(NCCCN1CCCC1=O)c1nc2ccccc2[nH]c1=O] | 46931 | 10-30 | >100 |
| 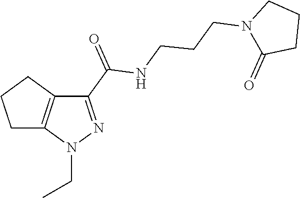 [SMILES: CCn1nc(C(=O)NCCCN2CCCC2=O)c2c1CCC2] | 46932 | >100 | >100 |
| 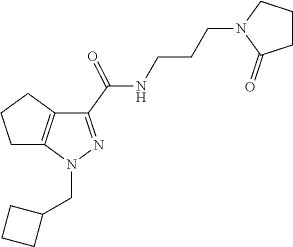 [SMILES: O=C(NCCCN1CCCC1=O)c1nn(CC2CCC2)c2c1CCC2] | 46933 | >100 | >100 |
| 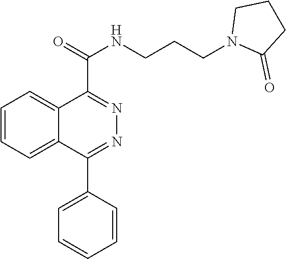 [SMILES: O=C(NCCCN1CCCC1=O)c1nnc(-c2ccccc2)c2ccccc12] | 46934 | >100 | >100 |
| 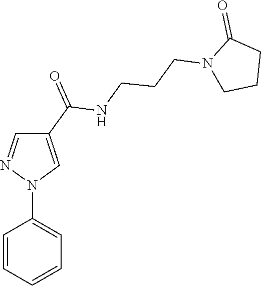 [SMILES: O=C(NCCCN1CCCC1=O)c1cnn(-c2ccccc2)c1] | 46935 | >100 | >100 |
| 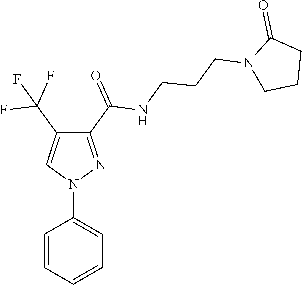 [SMILES: O=C(NCCCN1CCCC1=O)c1nn(-c2ccccc2)cc1C(F)(F)F] | 46979 | 50-100 | >100 |
| 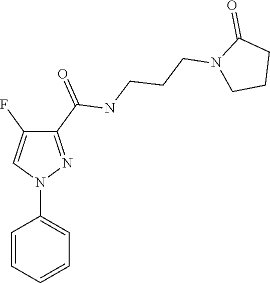 [SMILES: O=C(NCCCN1CCCC1=O)c1nn(-c2ccccc2)cc1F] | 47010 | >100 | >100 |
| 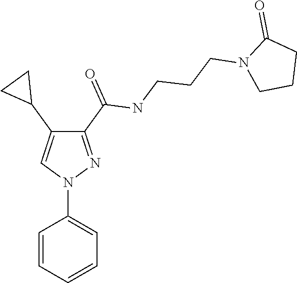 [SMILES: O=C(NCCCN1CCCC1=O)c1nn(-c2ccccc2)cc1C1CC1] | 47011 | >100 | >100 |
| 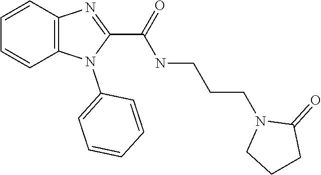 [SMILES: O=C(NCCCN1CCCC1=O)c1nc2ccccc2n1-c1ccccc1] | 47051 | >25 | >25 |
| 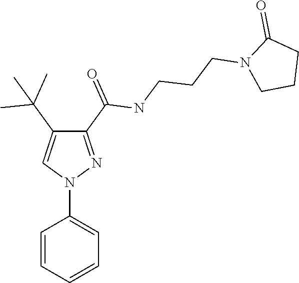 [SMILES: CC(C)(C)c1cn(-c2ccccc2)nc1C(=O)NCCCN1CCCC1=O] | 47091 | >100 | 50-100 |
| 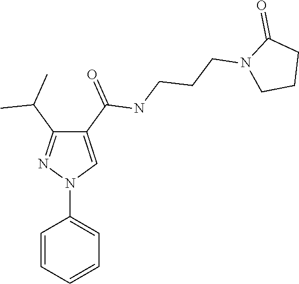 [SMILES: CC(C)c1nn(-c2ccccc2)cc1C(=O)NCCCN1CCCC1=O] | 47092 | >100 | >100 |
| 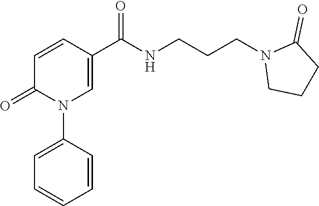 [SMILES: O=C(NCCCN1CCCC1=O)c1ccc(=O)n(-c2ccccc2)c1] | 47155 | >100 | >100 |
| 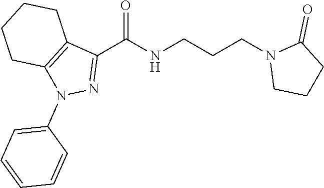 [SMILES: O=C(NCCCN1CCCC1=O)c1nn(-c2ccccc2)c2c1CCCC2] | GB-02 | 50-100 | >100 |
| 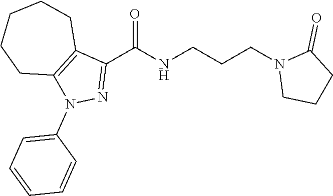 [SMILES: O=C(NCCCN1CCCC1=O)c1nn(-c2ccccc2)c2c1CCCCC2] | GB-03 | >100 | >100 |
| 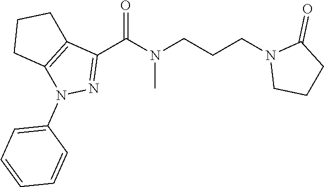 [SMILES: CN(CCCN1CCCC1=O)C(=O)c1nn(-c2ccccc2)c2c1CCC2] | GB-04 | >100 | >100 |
| 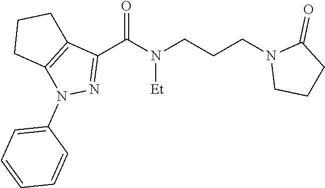 [SMILES: CCN(CCCN1CCCC1=O)C(=O)c1nn(-c2ccccc2)c2c1CCC2] | GB-05 | >100 | >100 |
| 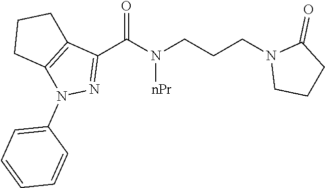 | GB-06 | 5-30 | >100 |
| 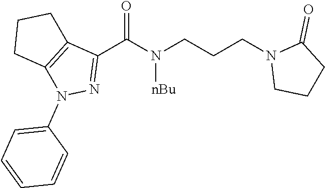 | GB-07 | 50-100 | 50-100 |
| 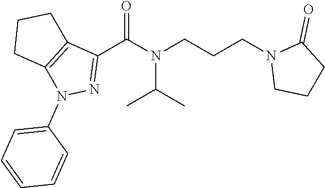 [SMILES: CC(C)N(CCCN1CCCC1=O)C(=O)c1nn(-c2ccccc2)c2c1CCC2] | GB-08 | 30-50 | >100 |
| 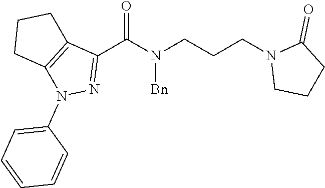 [SMILES: O=C1CCCN1CCCN(Cc1ccccc1)C(=O)c1nn(-c2ccccc2)c2c1CCC2] | GB-09 | 25-50 | 25-50 |
| 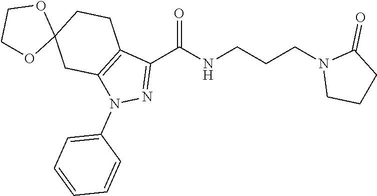 [SMILES: O=C(NCCCN1CCCC1=O)c1nn(-c2ccccc2)c2c1CCC1(C2)OCCO1] | GB-10 | >100 | >100 |
| 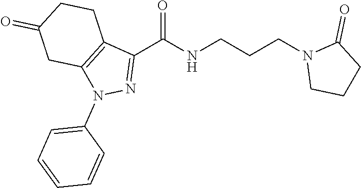 [SMILES: O=C1CCc2c(C(=O)NCCCN3CCCC3=O)nn(-c3ccccc3)c2C1] | GB-11 | 10-20 | >100 |
| 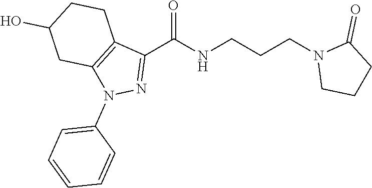 [SMILES: O=C(NCCCN1CCCC1=O)c1nn(-c2ccccc2)c2c1CCC(O)C2] | GB-12 | 1-10 | >100 |
| 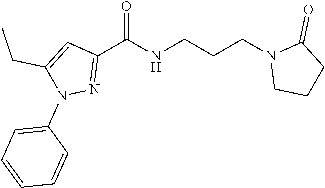 [SMILES: CCc1cc(C(=O)NCCCN2CCCC2=O)nn1-c1ccccc1] | GB-13 | 10-20 | >100 |
| 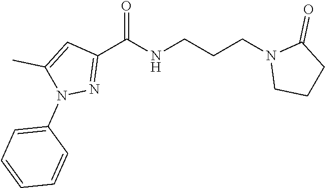 [SMILES: Cc1cc(C(=O)NCCCN2CCCC2=O)nn1-c1ccccc1] | GB-14 | >100 | >100 |
| 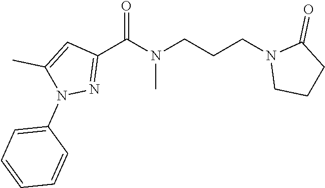 [SMILES: Cc1cc(C(=O)N(C)CCCN2CCCC2=O)nn1-c1ccccc1] | GB-15 | >100 | >100 |
| 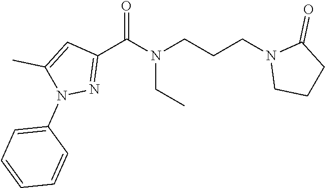 [SMILES: CCN(CCCN1CCCC1=O)C(=O)c1cc(C)n(-c2ccccc2)n1] | GB-16 | >100 | >100 |
| 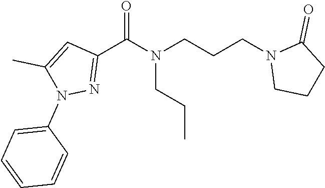 [SMILES: CCCN(CCCN1CCCC1=O)C(=O)c1cc(C)n(-c2ccccc2)n1] | GB-17 | >100 | >100 |
| 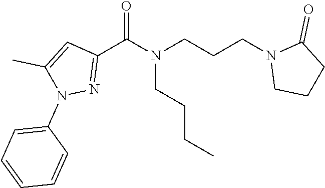 [SMILES: CCCCN(CCCN1CCCC1=O)C(=O)c1cc(C)n(-c2ccccc2)n1] | GB-18 | | |

TABLE 2
Tat protein inhibition for analogs of TT-44863 evaluated using the reporter cell line
HeLa-CD4-LTR-LacZ.

| | | Infectivity IC50 | Infectivity CC50 |
|---|---|---|---|
| Compound Structure | ID | (uM) | (uM) |
| 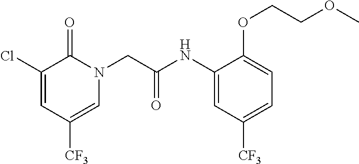 [SMILES: COCCOc1ccc(C(F)(F)F)cc1NC(=O)Cn1cc(C(F)(F)F)cc(Cl)c1=O] | 44863 | 1-10 | >800 |
| 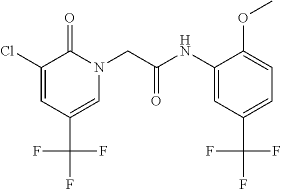 [SMILES: COc1ccc(C(F)(F)F)cc1NC(=O)Cn1cc(C(F)(F)F)cc(Cl)c1=O] | 46600 | 1-10 | >100 |
| 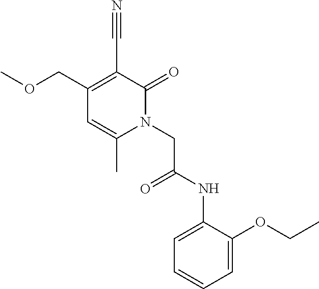 [SMILES: CCOc1ccccc1NC(=O)Cn1c(C)cc(COC)c(C#N)c1=O] | 46602 | >100 | >100 |
| 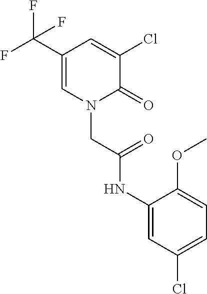 [SMILES: COc1ccc(Cl)cc1NC(=O)Cn1cc(C(F)(F)F)cc(Cl)c1=O] | 46603 | 20-30 | 50-100 |
| 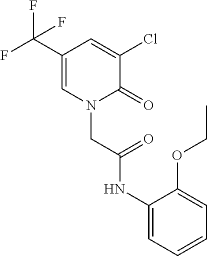 [SMILES: CCOc1ccccc1NC(=O)Cn1cc(C(F)(F)F)cc(Cl)c1=O] | 46604 | >100 | >100 |
| 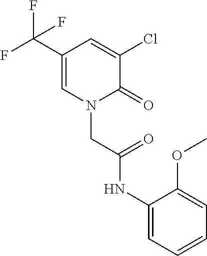 [SMILES: COc1ccccc1NC(=O)Cn1cc(C(F)(F)F)cc(Cl)c1=O] | 46605 | >100 | >100 |
| 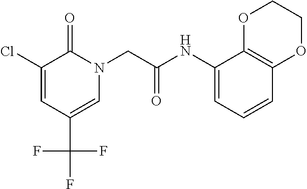 [SMILES: O=C(Cn1cc(C(F)(F)F)cc(Cl)c1=O)Nc1cccc2c1OCCO2] | 46615 | >100 | >100 |
| 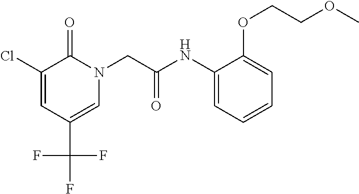 [SMILES: COCCOc1ccccc1NC(=O)Cn1cc(C(F)(F)F)cc(Cl)c1=O] | 46624 | >100 | >100 |
| 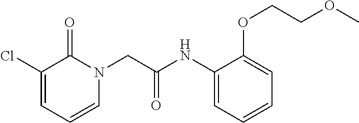 [SMILES: COCCOc1ccccc1NC(=O)Cn1cccc(Cl)c1=O] | 46631 | >100 | >100 |
| 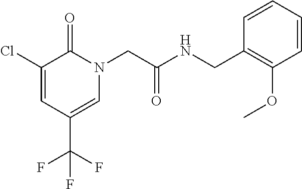 [SMILES: COc1ccccc1CNC(=O)Cn1cc(C(F)(F)F)cc(Cl)c1=O] | 46688 | >100 | >100 |
| 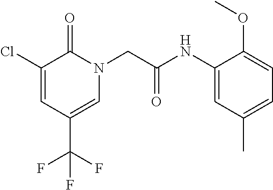 [SMILES: COc1ccc(C)cc1NC(=O)Cn1cc(C(F)(F)F)cc(Cl)c1=O] | 46689 | >100 | >100 |
| 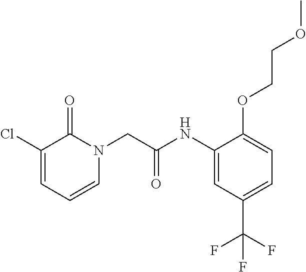 [SMILES: COCCOc1ccc(C(F)(F)F)cc1NC(=O)Cn1cccc(Cl)c1=O] | 46704 | >100 | >100 |
| 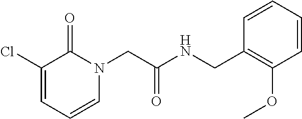 [SMILES: COc1ccccc1CNC(=O)Cn1cccc(Cl)c1=O] | 46747 | >100 | >100 |
| 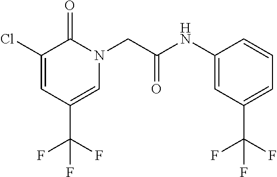 [SMILES: O=C(Cn1cc(C(F)(F)F)cc(Cl)c1=O)Nc1cccc(C(F)(F)F)c1] | 46748 | >100 | >100 |
| 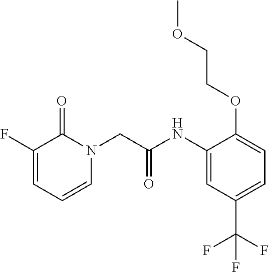 [SMILES: COCCOc1ccc(C(F)(F)F)cc1NC(=O)Cn1cccc(F)c1=O] | 46762 | >100 | >100 |
| 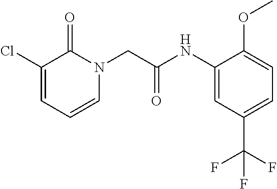 [SMILES: COc1ccc(C(F)(F)F)cc1NC(=O)Cn1cccc(Cl)c1=O] | 46763 | 50-100 | >100 |
| 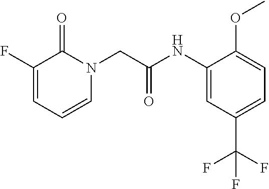 [SMILES: COc1ccc(C(F)(F)F)cc1NC(=O)Cn1cccc(F)c1=O] | 46765 | >100 | >100 |
| 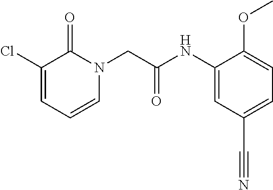 [SMILES: COc1ccc(C#N)cc1NC(=O)Cn1cccc(Cl)c1=O] | 46766 | >100 | >100 |
| 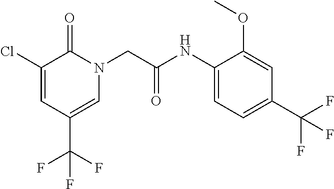 [SMILES: COc1cc(C(F)(F)F)ccc1NC(=O)Cn1cc(C(F)(F)F)cc(Cl)c1=O] | 46808 | 1-30 | >100 |
| 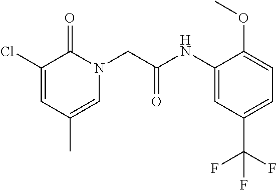 [SMILES: COc1ccc(C(F)(F)F)cc1NC(=O)Cn1cc(C)cc(Cl)c1=O] | 46809 | 1-10 | >100 |
| 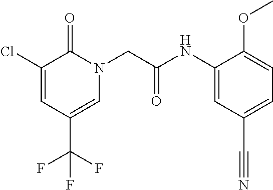 [SMILES: COc1ccc(C#N)cc1NC(=O)Cn1cc(C(F)(F)F)cc(Cl)c1=O] | 46828 | 50-100 | 50-100 |
| 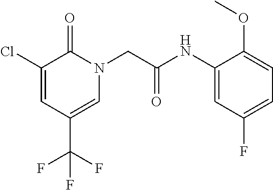 [SMILES: COc1ccc(F)cc1NC(=O)Cn1cc(C(F)(F)F)cc(Cl)c1=O] | 46867 | 10-30 | 50-100 |
| 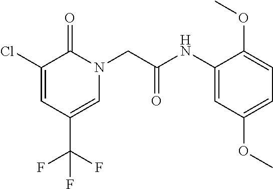 [SMILES: COc1ccc(OC)c(NC(=O)Cn2cc(C(F)(F)F)cc(Cl)c2=O)c1] | 46868 | >100 | >100 |
| 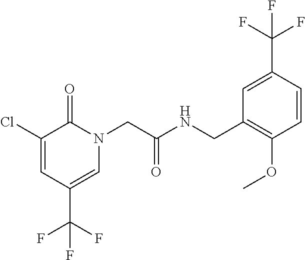 [SMILES: COc1ccc(C(F)(F)F)cc1CNC(=O)Cn1cc(C(F)(F)F)cc(Cl)c1=O] | 46875 | >100 | >100 |
| 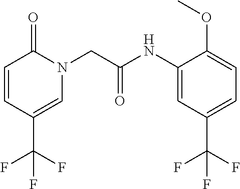 [SMILES: COc1ccc(C(F)(F)F)cc1NC(=O)Cn1cc(C(F)(F)F)ccc1=O] | 46917 | 1-30 | >100 |
| 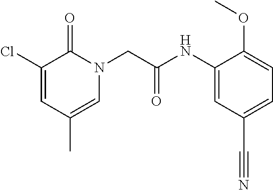 [SMILES: COc1ccc(C#N)cc1NC(=O)Cn1cc(C)cc(Cl)c1=O] | 46946 | >50 | >50 |
| 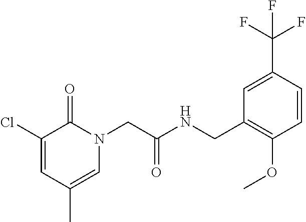 [SMILES: COc1ccc(C(F)(F)F)cc1CNC(=O)Cn1cc(C)cc(Cl)c1=O] | 46957 | >50 | >50 |
| 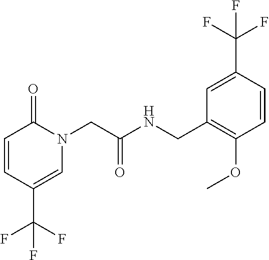 [SMILES: COc1ccc(C(F)(F)F)cc1CNC(=O)Cn1cc(C(F)(F)F)ccc1=O] | 46960 | >100 | >100 |
| 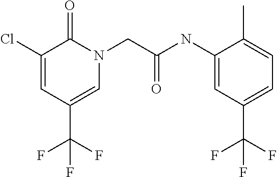 [SMILES: Cc1ccc(C(F)(F)F)cc1NC(=O)Cn1cc(C(F)(F)F)cc(Cl)c1=O] | 47001 | 1-10 | >100 |
| 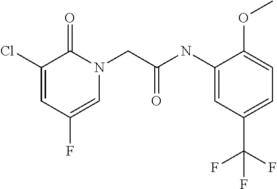 [SMILES: COc1ccc(C(F)(F)F)cc1NC(=O)Cn1cc(F)cc(Cl)c1=O] | 47006 | >50 | >50 |
| 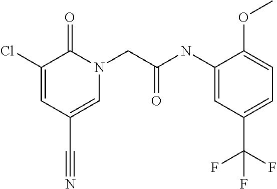 [SMILES: COc1ccc(C(F)(F)F)cc1NC(=O)Cn1cc(C#N)cc(Cl)c1=O] | 47018 | >50 | >50 |
| 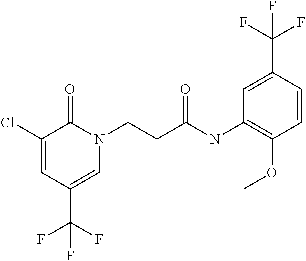 [SMILES: COc1ccc(C(F)(F)F)cc1NC(=O)CCn1cc(C(F)(F)F)cc(Cl)c1=O] | 47020 | >25 | >25 |
| 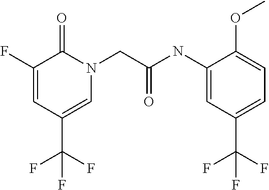 [SMILES: COc1ccc(C(F)(F)F)cc1NC(=O)Cn1cc(C(F)(F)F)cc(F)c1=O] | 47033 | 1-10 | 50-100 |
| 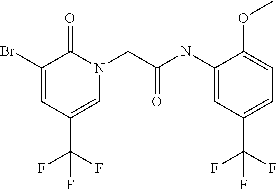 [SMILES: COc1ccc(C(F)(F)F)cc1NC(=O)Cn1cc(C(F)(F)F)cc(Br)c1=O] | 47055 | 1-10 | 50-100 |
| 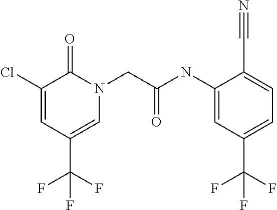 [SMILES: N#Cc1ccc(C(F)(F)F)cc1NC(=O)Cn1cc(C(F)(F)F)cc(Cl)c1=O] | 47061 | >100 | >100 |
| 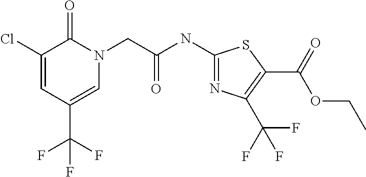 [SMILES: CCOC(=O)c1sc(NC(=O)Cn2cc(C(F)(F)F)cc(Cl)c2=O)nc1C(F)(F)F] | 47063 | 23.71 | 25-50 |
| 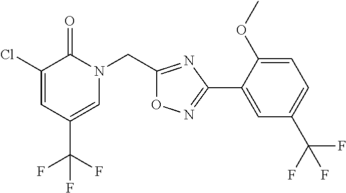 [SMILES: COc1ccc(C(F)(F)F)cc1-c1noc(Cn2cc(C(F)(F)F)cc(Cl)c2=O)n1] | 47111 | >100 | >100 |
| 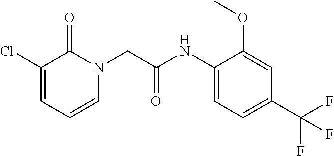 [SMILES: COc1cc(C(F)(F)F)ccc1NC(=O)Cn1cccc(Cl)c1=O] | 47171 | >100 | >100 |
| 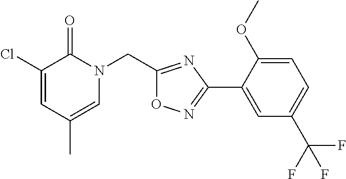 [SMILES: COc1ccc(C(F)(F)F)cc1-c1noc(Cn2cc(C)cc(Cl)c2=O)n1] | 47198 | >50 | >50 |
| 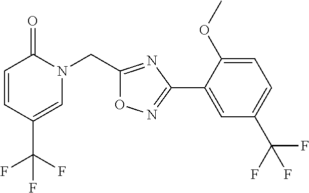 [SMILES: COc1ccc(C(F)(F)F)cc1-c1noc(Cn2cc(C(F)(F)F)ccc2=O)n1] | 47199 | >50 | >50 |
| 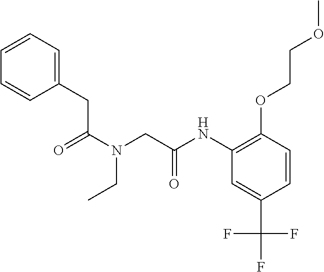 [SMILES: CCN(CC(=O)Nc1cc(C(F)(F)F)ccc1OCCOC)C(=O)Cc1ccccc1] | 45172 | ~80 | ~80 |
| 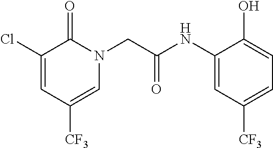 [SMILES: O=C(Cn1cc(C(F)(F)F)cc(Cl)c1=O)Nc1cc(C(F)(F)F)ccc1O] | | | |
| 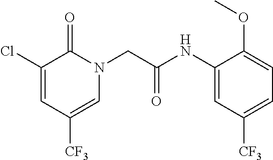 [SMILES: COc1ccc(C(F)(F)F)cc1NC(=O)Cn1cc(C(F)(F)F)cc(Cl)c1=O] | | | |
| 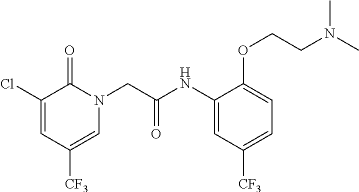 [SMILES: CN(C)CCOc1ccc(C(F)(F)F)cc1NC(=O)Cn1cc(C(F)(F)F)cc(Cl)c1=O] | | | |
| 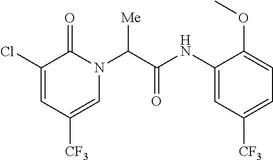 [SMILES: COc1ccc(C(F)(F)F)cc1NC(=O)C(C)n1cc(C(F)(F)F)cc(Cl)c1=O] | | 1-20 | >100 |
| 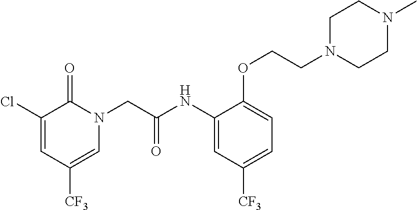 [SMILES: CN1CCN(CCOc2ccc(C(F)(F)F)cc2NC(=O)Cn2cc(C(F)(F)F)cc(Cl)c2=O)CC1] | | 10-20 | 25-50 |
| 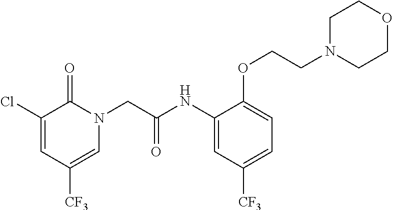 [SMILES: O=C(Cn1cc(C(F)(F)F)cc(Cl)c1=O)Nc1cc(C(F)(F)F)ccc1OCCN1CCOCC1] | | 1-10 | 50-100 |
| 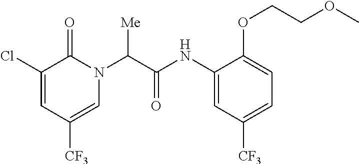 [SMILES: COCCOc1ccc(C(F)(F)F)cc1NC(=O)C(C)n1cc(C(F)(F)F)cc(Cl)c1=O] | | | |
| 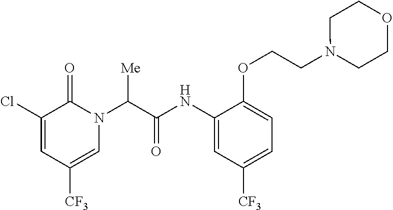 [SMILES: CC(C(=O)Nc1cc(C(F)(F)F)ccc1OCCN1CCOCC1)n1cc(C(F)(F)F)cc(Cl)c1=O] | | | |
| 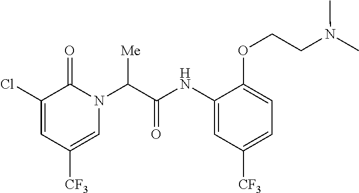 [SMILES: CC(C(=O)Nc1cc(C(F)(F)F)ccc1OCCN(C)C)n1cc(C(F)(F)F)cc(Cl)c1=O] | | | |
| 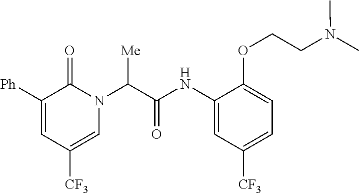 [SMILES: CC(C(=O)Nc1cc(C(F)(F)F)ccc1OCCN(C)C)n1cc(C(F)(F)F)cc(-c2ccccc2)c1=O] | | | |
| 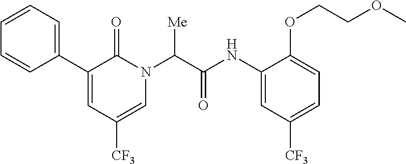 [SMILES: COCCOc1ccc(C(F)(F)F)cc1NC(=O)C(C)n1cc(C(F)(F)F)cc(-c2ccccc2)c1=O] | | | |
| 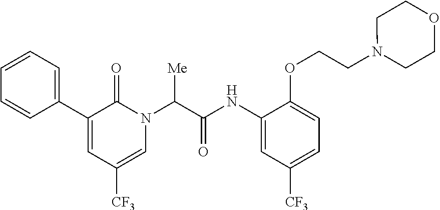 [SMILES: CC(C(=O)Nc1cc(C(F)(F)F)ccc1OCCN1CCOCC1)n1cc(C(F)(F)F)cc(-c2ccccc2)c1=O] | | | |
| 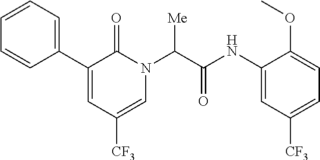 [SMILES: COc1ccc(C(F)(F)F)cc1NC(=O)C(C)n1cc(C(F)(F)F)cc(-c2ccccc2)c1=O] | | >100 | >100 |
| 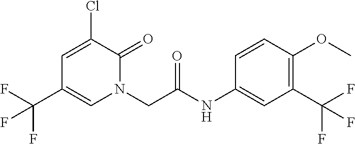 [SMILES: COc1ccc(NC(=O)Cn2cc(C(F)(F)F)cc(Cl)c2=O)cc1C(F)(F)F] | 44 | >100 | >100 |
| 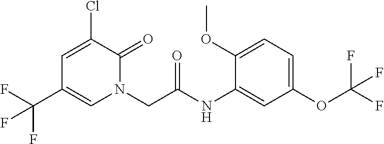 [SMILES: COc1ccc(OC(F)(F)F)cc1NC(=O)Cn1cc(C(F)(F)F)cc(Cl)c1=O] | 45 | 1-10 | >100 |
| 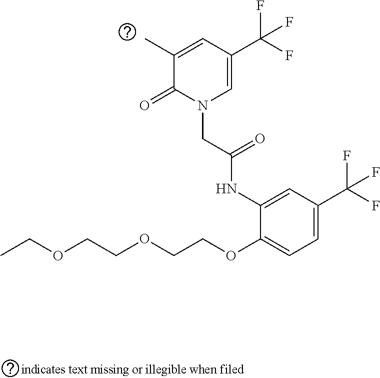 [SMILES: CCOCCOCCOc1ccc(C(F)(F)F)cc1NC(=O)Cn1cc(C(F)(F)F)cc(C)c1=O] | 46 | 1-10 | >100 |
| 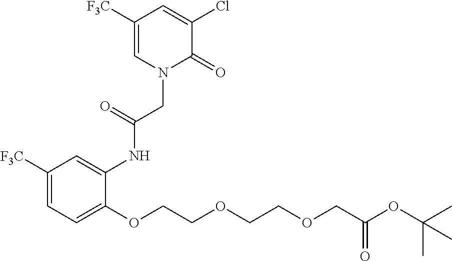 [SMILES: CC(C)(C)OC(=O)COCCOCCOc1ccc(C(F)(F)F)cc1NC(=O)Cn1cc(C(F)(F)F)cc(Cl)c1=O] | 47 | 50-100 | >100 |
| 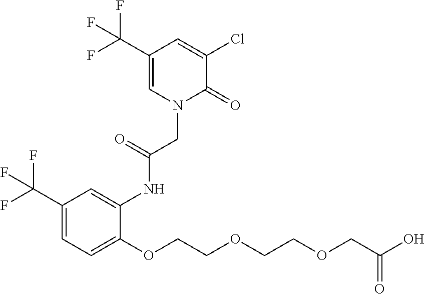 [SMILES: O=C(O)COCCOCCOc1ccc(C(F)(F)F)cc1NC(=O)Cn1cc(C(F)(F)F)cc(Cl)c1=O] | 48 | >100 | >100 |
| 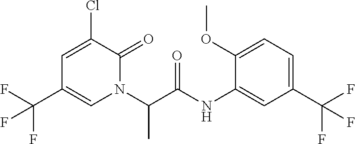 [SMILES: COc1ccc(C(F)(F)F)cc1NC(=O)C(C)n1cc(C(F)(F)F)cc(Cl)c1=O] | 49 | 1-20 | >100 |
| 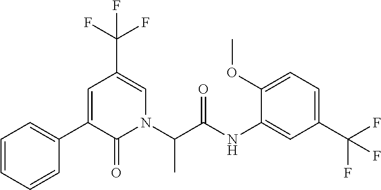 [SMILES: COc1ccc(C(F)(F)F)cc1NC(=O)C(C)n1cc(C(F)(F)F)cc(-c2ccccc2)c1=O] | 50 | >100 | >100 |
| 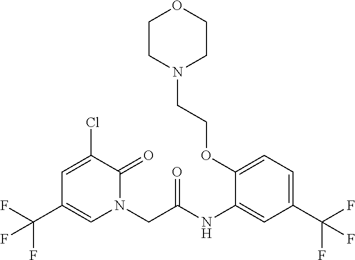 [SMILES: O=C(Cn1cc(C(F)(F)F)cc(Cl)c1=O)Nc1cc(C(F)(F)F)ccc1OCCN1CCOCC1] | 51 | 1-10 | 50-100 |
| 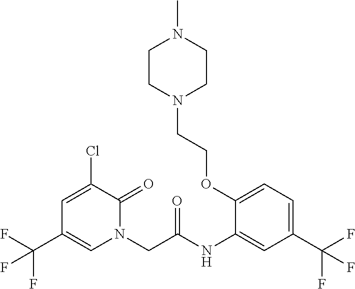 [SMILES: CN1CCN(CCOc2ccc(C(F)(F)F)cc2NC(=O)Cn2cc(C(F)(F)F)cc(Cl)c2=O)CC1] | 52 | 10-20 | 25-50 |
| 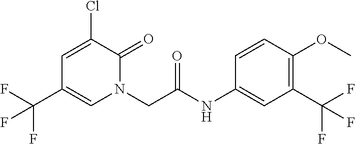 [SMILES: COc1ccc(NC(=O)Cn2cc(C(F)(F)F)cc(Cl)c2=O)cc1C(F)(F)F] | 53 | >100 uM | >100 uM |
| 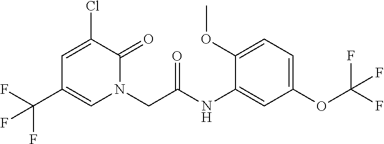 [SMILES: COc1ccc(OC(F)(F)F)cc1NC(=O)Cn1cc(C(F)(F)F)cc(Cl)c1=O] | 54 | 1-10 | >100 |
| 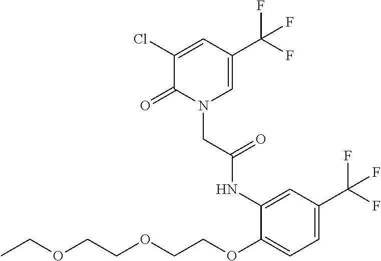 [SMILES: CCOCCOCCOc1ccc(C(F)(F)F)cc1NC(=O)Cn1cc(C(F)(F)F)cc(Cl)c1=O] | 55 | 1-20 | >100 |
| 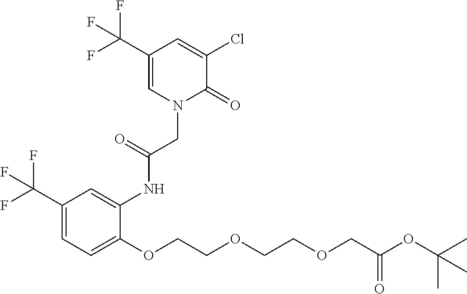 [SMILES: CC(C)(C)OC(=O)COCCOCCOc1ccc(C(F)(F)F)cc1NC(=O)Cn1cc(C(F)(F)F)cc(Cl)c1=O] | 56 | >100 | >100 |
| 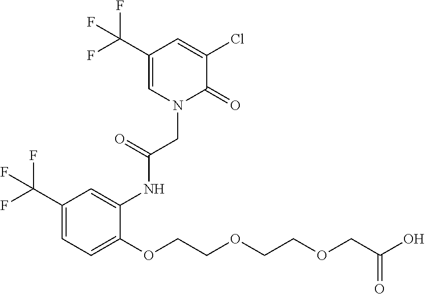 [SMILES: O=C(O)COCCOCCOc1ccc(C(F)(F)F)cc1NC(=O)Cn1cc(C(F)(F)F)cc(Cl)c1=O] | 57 | >100 | >100 |
| 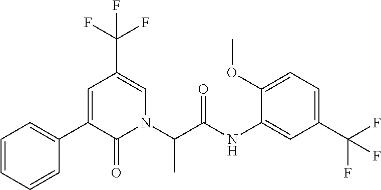 [SMILES: COc1ccc(C(F)(F)F)cc1NC(=O)C(C)n1cc(C(F)(F)F)cc(-c2ccccc2)c1=O] | 59 | >100 | >100 |
| 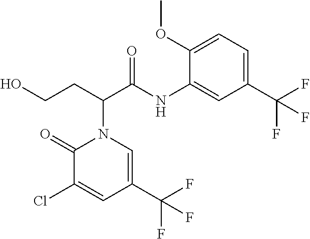 [SMILES: COc1ccc(C(F)(F)F)cc1NC(=O)C(CCO)n1cc(C(F)(F)F)cc(Cl)c1=O] | 60 | 10-50 | 10-30 |
| 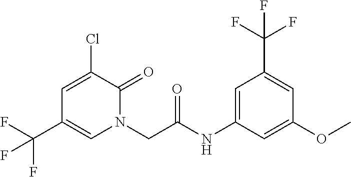 [SMILES: COc1cc(NC(=O)Cn2cc(C(F)(F)F)cc(Cl)c2=O)cc(C(F)(F)F)c1] | 61 | >100 | >100 |

TABLE 3
Tat protein inhibition for analogs of TT-44856 evaluated using the reporter cell line
HeLa-CD4-LTR-LacZ.

| | | Infectivity | Infectivity |
|---|---|---|---|
| Compound Structure | ID | IC₅₀ (uM) | CC₅₀ (uM) |
| 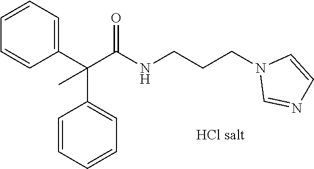 [SMILES: CC(C(=O)NCCCn1ccnc1)(c1ccccc1)c1ccccc1] | 44856 | 1-20 | >100 |
| 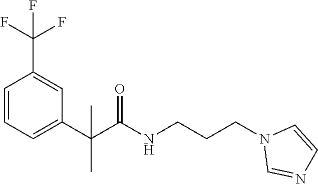 [SMILES: CC(C)(C(=O)NCCCn1ccnc1)c1cccc(C(F)(F)F)c1] | 46606 | >100 | >100 |
| 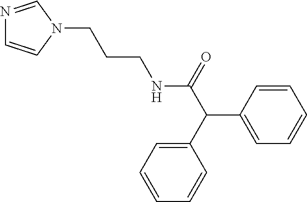 [SMILES: O=C(NCCCn1ccnc1)C(c1ccccc1)c1ccccc1] | 46608 | 1-10 | >100 |
| 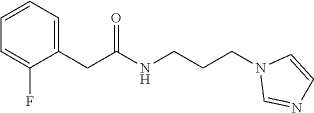 [SMILES: O=C(Cc1ccccc1F)NCCCn1ccnc1] | Z31795827 | >100 | N.D |
| 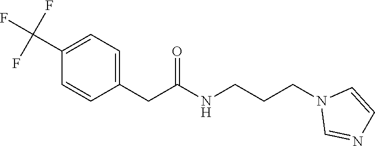 [SMILES: O=C(Cc1ccc(C(F)(F)F)cc1)NCCCn1ccnc1] | Z236749082 | >100 | N.D |
| 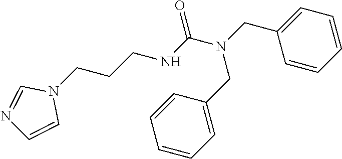 [SMILES: O=C(NCCCn1ccnc1)N(Cc1ccccc1)Cc1ccccc1] | Z991981670 | >100 | N.D |
| 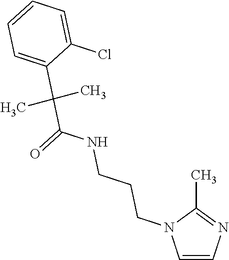 [SMILES: Cc1nccn1CCCNC(=O)C(C)(C)c1ccccc1Cl] | Z1119105307 | >100 | N.D |
| 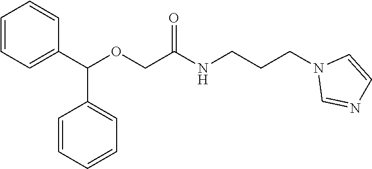 [SMILES: O=C(COC(c1ccccc1)c1ccccc1)NCCCn1ccnc1] | Z51979572 | >100 | N.D |
| 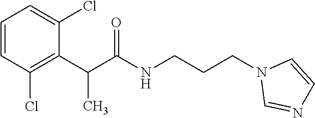 [SMILES: CC(C(=O)NCCCn1ccnc1)c1c(Cl)cccc1Cl] | Z1004969394 | >100 | N.D |
| 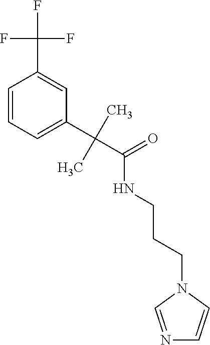 [SMILES: CC(C)(C(=O)NCCCn1ccnc1)c1cccc(C(F)(F)F)c1] | Z1053536312 | >100 | N.D |
| 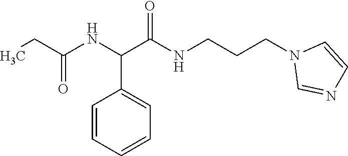 [SMILES: CCC(=O)NC(C(=O)NCCCn1ccnc1)c1ccccc1] | Z1207330250 | >100 | N.D |
| 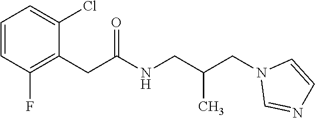 [SMILES: CC(CNC(=O)Cc1c(F)cccc1Cl)Cn1ccnc1] | Z1250741002 | >100 | N.D |
| 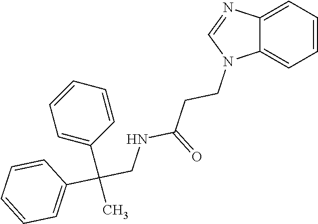 [SMILES: CC(CNC(=O)CCn1cnc2ccccc21)(c1ccccc1)c1ccccc1] | Z119886832 | >100 | N.D |
| 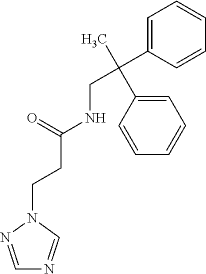 [SMILES: CC(CNC(=O)CCn1cncn1)(c1ccccc1)c1ccccc1] | Z221503086 | >100 | N.D |
| 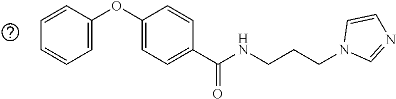 [SMILES: O=C(NCCCn1ccnc1)c1ccc(Oc2ccccc2)cc1] | Z56813845 | >100 | N.D |
| 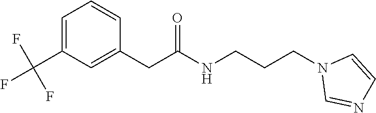 [SMILES: O=C(Cc1cccc(C(F)(F)F)c1)NCCCn1ccnc1] | Z30217436 | >100 | N.D |
| 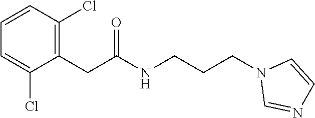 [SMILES: O=C(Cc1c(Cl)cccc1Cl)NCCCn1ccnc1] | Z30217435 | >100 | N.D |
| 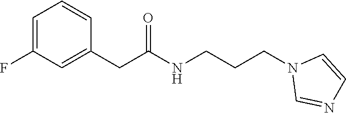 [SMILES: O=C(Cc1cccc(F)c1)NCCCn1ccnc1] | Z31796183 | >100 | N.D |
| 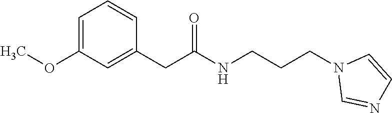 [SMILES: COc1cccc(CC(=O)NCCCn2ccnc2)c1] | Z31796092 | >100 | N.D |
| 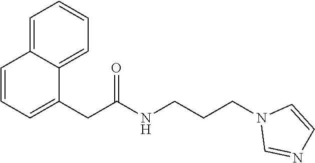 [SMILES: O=C(Cc1cccc2ccccc12)NCCCn1ccnc1] | Z31795406 | >100 | N.D |
| 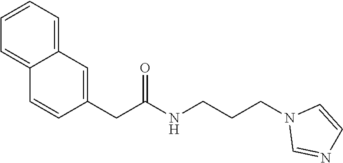 [SMILES: O=C(Cc1ccc2ccccc2c1)NCCCn1ccnc1] | Z31795124 | >100 | N.D |
| 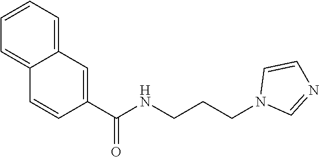 [SMILES: O=C(NCCCn1ccnc1)c1ccc2ccccc2c1] | Z31795639 | >100 | N.D |
| 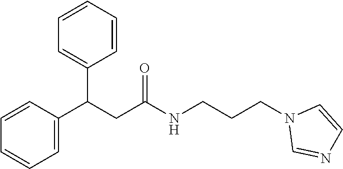 [SMILES: O=C(CC(c1ccccc1)c1ccccc1)NCCCn1ccnc1] | Z30217483 | >100 | N.D |
| 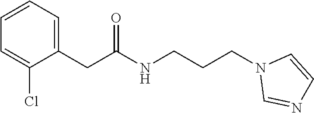 [SMILES: O=C(Cc1ccccc1Cl)NCCCn1ccnc1] | Z31796195 | >100 | N.D |
| 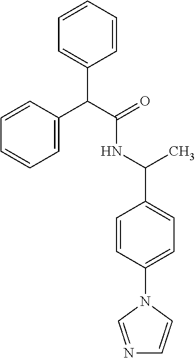 [SMILES: CC(NC(=O)C(c1ccccc1)c1ccccc1)c1ccc(-n2ccnc2)cc1] | Z27307031 | 1-20 | >100 |
| 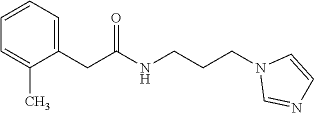 [SMILES: Cc1ccccc1CC(=O)NCCCn1ccnc1] | Z51979592 | >100 | N.D |
| 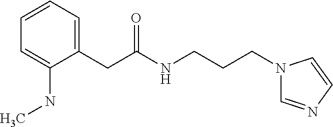 [SMILES: CNc1ccccc1CC(=O)NCCCn1ccnc1] | Z31794405 | >100 | N.D |
| 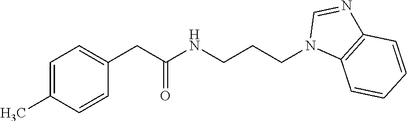 [SMILES: Cc1ccc(CC(=O)NCCCn2cnc3ccccc32)cc1] | Z102452176 | 10-30 | >100 |
| 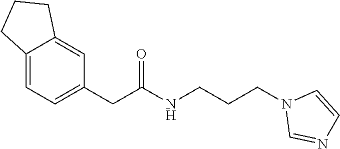 [SMILES: O=C(Cc1ccc2c(c1)CCC2)NCCCn1ccnc1] | Z51979499 | >100 | N.D |
| 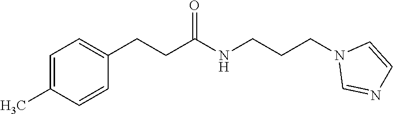 [SMILES: Cc1ccc(CCC(=O)NCCCn2ccnc2)cc1] | Z196426172 | >100 | N.D |
| 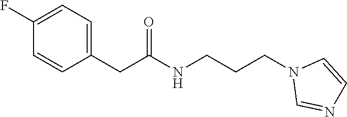 [SMILES: O=C(Cc1ccc(F)cc1)NCCCn1ccnc1] | Z31795779 | >100 | N.D |
| 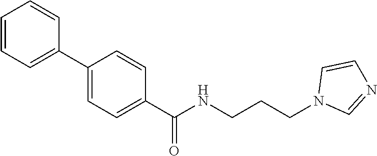 [SMILES: O=C(NCCCn1ccnc1)c1ccc(-c2ccccc2)cc1] | Z31795699 | >100 | N.D |
| 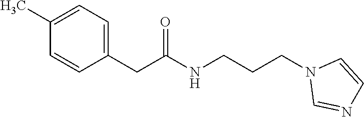 [SMILES: Cc1ccc(CC(=O)NCCCn2ccnc2)cc1] | Z100612606 | >100 | N.D |
| 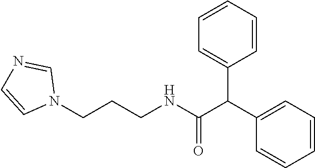 [SMILES: O=C(NCCCn1ccnc1)C(c1ccccc1)c1ccccc1] | Z30217425 | N.D | N.D |
| 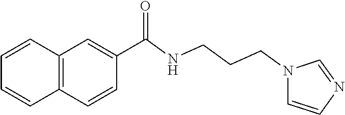 [SMILES: O=C(NCCCn1ccnc1)c1ccc2ccccc2c1] | 3-34 | >100 | >100 |
| 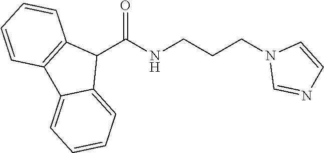 [SMILES: O=C(NCCCn1ccnc1)C1c2ccccc2-c2ccccc21] | 3- 35 | 50-100 | >100 |
| 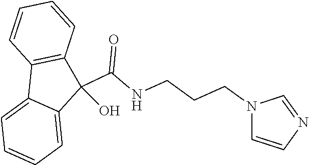 [SMILES: O=C(NCCCn1ccnc1)C1(O)c2ccccc2-c2ccccc21] | 3-36 | 20-50 | >100 |
| 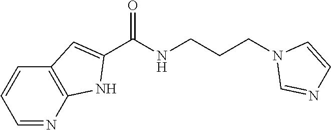 [SMILES: O=C(NCCCn1ccnc1)c1cc2cccnc2[nH]1] | 3-37 | >100 | >100 |
| 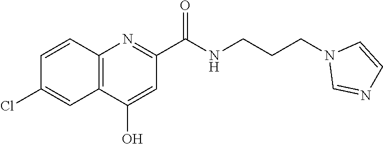 [SMILES: O=C(NCCCn1ccnc1)c1cc(O)c2cc(Cl)ccc2n1] | 3- 38 | >100 | >100 |
| 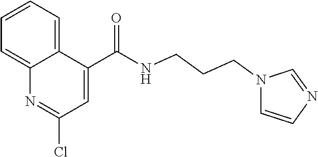 [SMILES: O=C(NCCCn1ccnc1)c1cc(Cl)nc2ccccc12] | 3-39 | >100 | >100 |
| 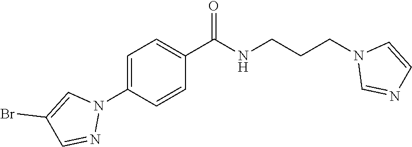 [SMILES: O=C(NCCCn1ccnc1)c1ccc(-n2cc(Br)cn2)cc1] | 3-40 | >100 | >100 |
| 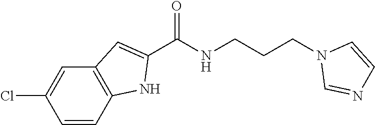 [SMILES: O=C(NCCCn1ccnc1)c1cc2cc(Cl)ccc2[nH]1] | 3-41 | >100 | >100 |
| 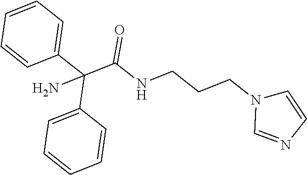 [SMILES: NC(C(=O)NCCCn1ccnc1)(c1ccccc1)c1ccccc1] | 3-42 | 10-30 | >100 |
| 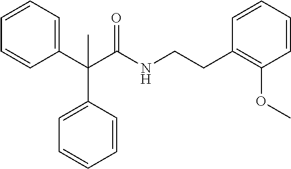 [SMILES: COc1ccccc1CCNC(=O)C(C)(c1ccccc1)c1ccccc1] | 3-43 | 20-100 | 25-50 |
| 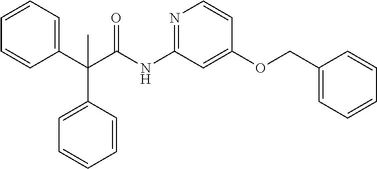 [SMILES: CC(C(=O)Nc1cc(OCc2ccccc2)ccn1)(c1ccccc1)c1ccccc1] | 3-44 | 1-30 | >100 |
| 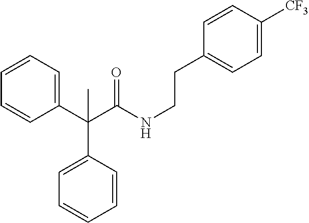 [SMILES: CC(C(=O)NCCc1ccc(C(F)(F)F)cc1)(c1ccccc1)c1ccccc1] | 3-45 | 10-20 | 50-100 |
| 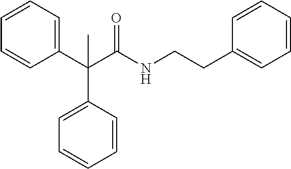 [SMILES: CC(C(=O)NCCc1ccccc1)(c1ccccc1)c1ccccc1] | 3-46 | 50-100 | 20-50 |
| 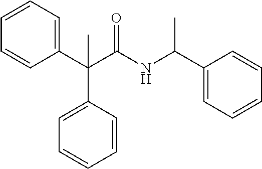 [SMILES: CC(NC(=O)C(C)(c1ccccc1)c1ccccc1)c1ccccc1] | 3-47 | 10-30 | >100 |
| 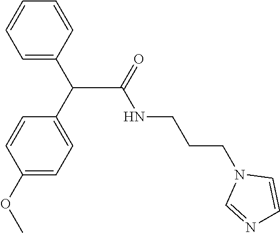 [SMILES: COc1ccc(C(C(=O)NCCCn2ccnc2)c2ccccc2)cc1] | 3-48 | 20-50 | >100 |
| 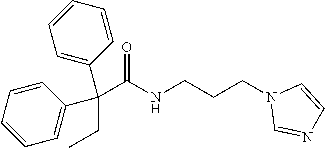 [SMILES: CCC(C(=O)NCCCn1ccnc1)(c1ccccc1)c1ccccc1] | 3-49 | 1-10 | >100 |
| 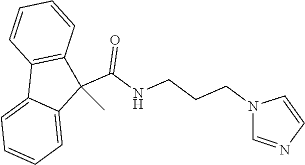 [SMILES: CC1(C(=O)NCCCn2ccnc2)c2ccccc2-c2ccccc21] | 3-50 | 1-20 | >100 |
| 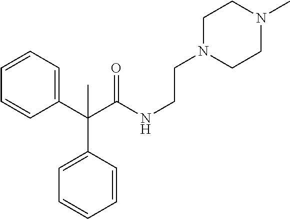 [SMILES: CN1CCN(CCNC(=O)C(C)(c2ccccc2)c2ccccc2)CC1] | 3-51 | >100 | >100 |
| 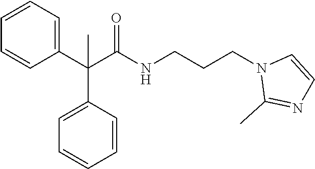 [SMILES: Cc1nccn1CCCNC(=O)C(C)(c1ccccc1)c1ccccc1] | 3-52 | 1-10 | >100 |
| 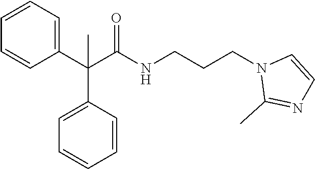 [SMILES: Cc1nccn1CCCNC(=O)C(C)(c1ccccc1)c1ccccc1] | 3-53 | 1-10 | 50-100 |
| 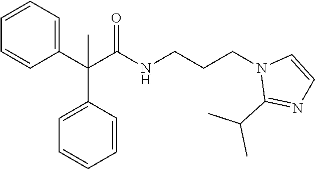 [SMILES: CC(C)c1nccn1CCCNC(=O)C(C)(c1ccccc1)c1ccccc1] | 3-54 | 1-20 | 50-100 |
| 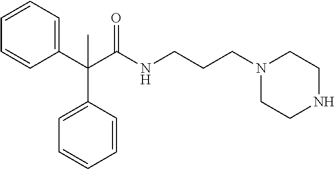 [SMILES: CC(C(=O)NCCCN1CCNCC1)(c1ccccc1)c1ccccc1] | 3-55 | >100 | >100 |
| 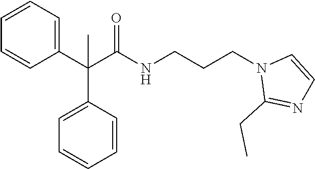 [SMILES: CCc1nccn1CCCNC(=O)C(C)(c1ccccc1)c1ccccc1] | 3-56 | 10-20 | >100 |
| 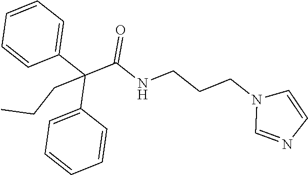 [SMILES: CCCC(C(=O)NCCCn1ccnc1)(c1ccccc1)c1ccccc1] | 3-57 | 1-10 | >100 |
| 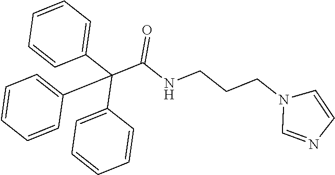 [SMILES: O=C(NCCCn1ccnc1)C(c1ccccc1)(c1ccccc1)c1ccccc1] | 3-58 | 1-20 | >100 |
| 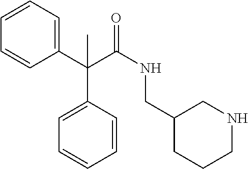 [SMILES: CC(C(=O)NCC1CCCNC1)(c1ccccc1)c1ccccc1] | 3-59 | 1-30 | >100 |
| 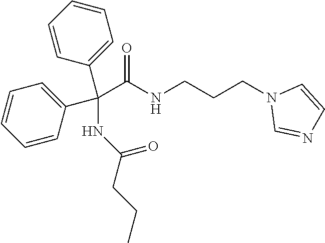 [SMILES: CCCC(=O)NC(C(=O)NCCCn1ccnc1)(c1ccccc1)c1ccccc1] | 3-60 | >100 | >100 |
| 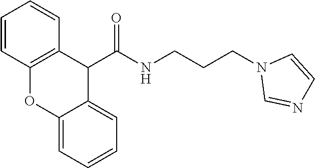 [SMILES: O=C(NCCCn1ccnc1)C1c2ccccc2Oc2ccccc21] | 3-61 | >100 | >100 |
| 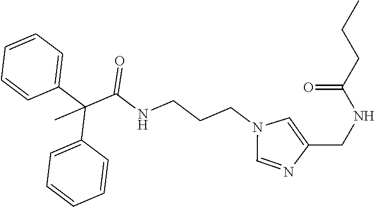 [SMILES: CCCC(=O)NCc1cn(CCCNC(=O)C(C)(c2ccccc2)c2ccccc2)cn1] | 3-62 | >100 | >100 |
| 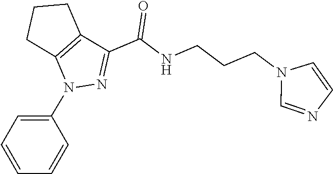 [SMILES: O=C(NCCCn1ccnc1)c1nn(-c2ccccc2)c2c1CCC2] | 3-63 | 1-10 | 1-10 |
| 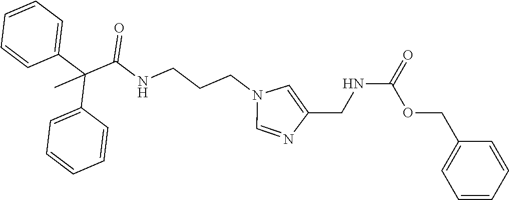 [SMILES: CC(C(=O)NCCCn1cnc(CNC(=O)OCc2ccccc2)c1)(c1ccccc1)c1ccccc1] | 3-64 | 10-30 | 20-50 |
| 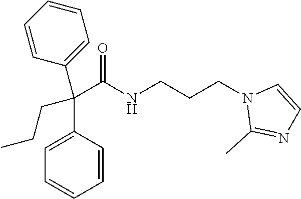 [SMILES: CCCC(C(=O)NCCCn1ccnc1C)(c1ccccc1)c1ccccc1] | 3-65 | 1-10 | 50-100 |
| 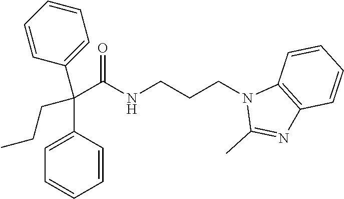 [SMILES: CCCC(C(=O)NCCCn1c(C)nc2ccccc21)(c1ccccc1)c1ccccc1] | 3-66 | 20-50 | 20-50 |
| 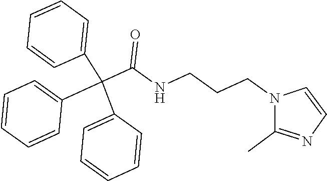 [SMILES: Cc1nccn1CCCNC(=O)C(c1ccccc1)(c1ccccc1)c1ccccc1] | 3-67 | 1-20 | 50-100 |
| 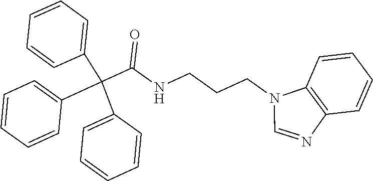 [SMILES: O=C(NCCCn1cnc2ccccc21)C(c1ccccc1)(c1ccccc1)c1ccccc1] | 3-68 | 10-30 | 20-50 |
| 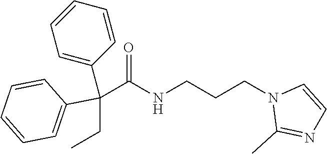 [SMILES: CCC(C(=O)NCCCn1ccnc1C)(c1ccccc1)c1ccccc1] | 3-69 | 1-10 | >100 |
| 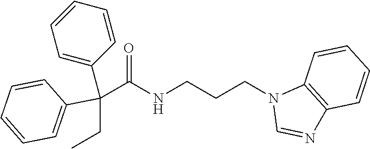 [SMILES: CCC(C(=O)NCCCn1cnc2ccccc21)(c1ccccc1)c1ccccc1] | 3-70 | 20-50 | 50-100 |
| 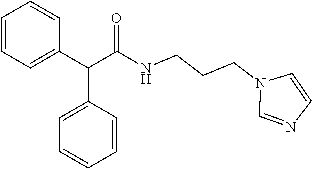 [SMILES: O=C(NCCCn1ccnc1)C(c1ccccc1)c1ccccc1] | 3-71 | 1-30 | >100 |
| 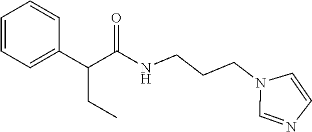 [SMILES: CCC(C(=O)NCCCn1ccnc1)c1ccccc1] | 3-72 | >100 | >100 |
| 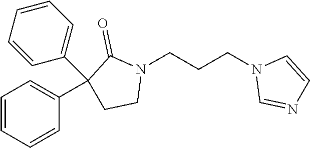 [SMILES: O=C1N(CCCn2ccnc2)CCC1(c1ccccc1)c1ccccc1] | 3-73 | 10-50 | >100 |
| 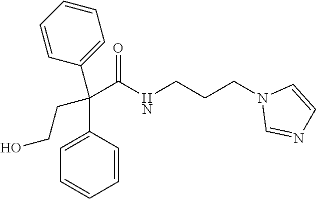 [SMILES: O=C(NCCCn1ccnc1)C(CCO)(c1ccccc1)c1ccccc1] | 3-74 | 1-20 | >100 |
| 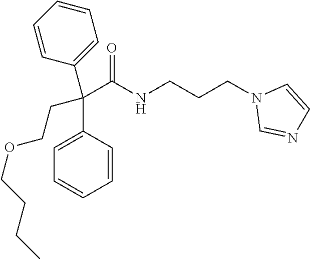 [SMILES: CCCCOCCC(C(=O)NCCCn1ccnc1)(c1ccccc1)c1ccccc1] | 3-75 | 50-100 | >100 |
| 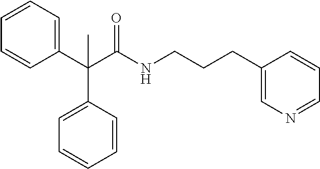 [SMILES: CC(C(=O)NCCCc1cccnc1)(c1ccccc1)c1ccccc1] | 3-76 | 1-30 | 10-30 |

### CONCLUSION

Three specific Tat degraders were identified upon HTS of 579443 small molecules in a cell-based Tat-TAR assay. TT-44863, TT-44881 and TT-44856 displayed good drug like properties and provided a starting point to develop analogs with improved antiviral activity. These compounds engage Cereblon to recognize Tat as a substrate and promote ubiquitination followed by proteasomal degradation. Their activity involves Tat Y26 residue in the cysteine rich region of Tat. Tat degradation by these molecules inhibits HIV acute infection without affecting cellular transcription and block HIV reactivation from different HIV latency models. These PROTAC type molecules may act via promotion of transient and reversible association with Tat and Cereblon. As such, one single molecule may induce degradation of more proteins sequentially, leading to sub-affinity equilibrium protein knockdown. Moreover, these types of molecules likely ensure longer efficacy time than small inhibitors since restoration of protein function requires protein resynthesis, reducing the need for high affinity target binding or long drug exposure. Unlike small molecule inhibitors, these do not require binding to catalytic site, or active site, since they mediate degradation by binding alternative non catalytic sites. Even targets defined as undruggable by occupancy-based ligands can be detected and degraded. Resistance to these types of drugs may also be less prominent given their ability to make transient interactions to induce a functional knock down of their target. However, other resistance mechanisms such as genomic differences in the ubiquitin-proteasome system may affect activity.

### REFERENCES

1 Hammer, S. M. et al. A controlled trial of two nucleoside analogues plus indinavir in persons with human immunodeficiency virus infection and CD4 cell counts of 200 per cubic millimeter or less. AIDS Clinical Trials Group 320 Study Team. N Engl J Med 337, 725-733, doi:10.1056/NEJM199709113371101 (1997).
2 Gulick, R. M. et al. Treatment with indinavir, zidovudine, and lamivudine in adults with human immunodeficiency virus infection and prior antiretroviral therapy. N Engl J Med 337, 734-739, doi:10.1056/NEJM199709113371102 (1997).
3 Sogaard, O. S. et al. The Depsipeptide Romidepsin Reverses HIV-1 Latency In Vivo. PLoS Pathog 11, e1005142, doi:10.1371/journal.ppat.1005142 (2015).
4 Chun, T. W. et al. Persistence of HIV in gut-associated lymphoid tissue despite long-term antiretroviral therapy. J Infect Dis 197, 714-720, doi:10.1086/527324 (2008).
5 Gunthard, H. F. et al. Residual human immunodeficiency virus (HIV) Type 1 RNA and DNA in lymph nodes and HIV RNA in genital secretions and in cerebrospinal fluid after suppression of viremia for 2 years. J Infect Dis 183, 1318-1327, doi:10.1086/319864 (2001).
6 Darcis, G., Van Driessche, B. & Van Lint, C. HIV Latency: Should We Shock or Lock? Trends Immunol 38, 217-228, doi:10.1016/j.it.2016.12.003 (2017).
7 Pitman, M. C., Lau, J. S. Y., McMahon, J. H. & Lewin, S. R. Barriers and strategies to achieve a cure for HIV. Lancet HIV 5, e317-e328, doi:10.1016/S2352-3018(18)30039-0 (2018).
8 Abner, E. & Jordan, A. HIV “shock and kill” therapy: In need of revision. Antiviral Res 166, 19-34, doi:10.1016/j.antiviral.2019.03.008 (2019).
9 Mousseau, G. et al. An analog of the natural steroidal alkaloid cortistatin A potently suppresses Tat-dependent HIV transcription. Cell Host Microbe 12, 97-108, doi:10.1016/j.chom.2012.05.016 (2012).
10 Mousseau, G. et al. The Tat Inhibitor Didehydro-Cortistatin A Prevents HIV-1 Reactivation from Latency. mBio 6, e00465, doi:10.1128/mBio.00465-15 (2015).
11 Kessing, C. F. et al. In Vivo Suppression of HIV Rebound by Didehydro-Cortistatin A, a “Block-and-Lock” Strategy for HIV-1 Treatment. Cell Rep 21, 600-611, doi:10.1016/j.celrep.2017.09.080 (2017).
12 Li, C., Mousseau, G. & Valente, S. T. Tat inhibition by didehydro-Cortistatin A promotes heterochromatin formation at the HIV-1 long terminal repeat. Epigenetics Chromatin 12, 23, doi:10.1186/s13072-019-0267-8 (2019).
13 Sodroski, J. et al. Trans-acting transcriptional regulation of human T-cell leukemia virus type III long terminal repeat. Science 227, 171-173 (1985).
14 Okamoto, T. & Wong-Staal, F. Demonstration of virus-specific transcriptional activator(s) in cells infected with HTLV-III by an in vitro cell-free system. Cell 47, 29-35 (1986).
15 Selby, M. J., Bain, E. S., Luciw, P. A. & Peterlin, B. M. Structure, sequence, and position of the stem-loop in tar determine transcriptional elongation by tat through the HIV-1 long terminal repeat. Genes Dev 3, 547-558 (1989).
16 Laspia, M. F., Rice, A. P. & Mathews, M. B. HIV-1 Tat protein increases transcriptional initiation and stabilizes elongation. Cell 59, 283-292, doi:0092-8674(89)90290-0 [pii](1989).
17 Mancebo, H. S. et al. P-TEFb kinase is required for HIV Tat transcriptional activation in vivo and in vitro. Genes Dev 11, 2633-2644 (1997).
18 Fujinaga, K. et al. The ability of positive transcription elongation factor B to transactivate human immunodeficiency virus transcription depends on a functional kinase domain, cyclin T1, and Tat. J Virol 72, 7154-7159 (1998).
19 Mediouni, S., Marcondes, M. C., Miller, C., McLaughlin, J. P. & Valente, S. T. The cross-talk of HIV-1 Tat and methamphetamine in HIV-associated neurocognitive disorders. Front Microbiol 6, 1164, doi:10.3389/fmicb.2015.01164 (2015).
20 Wender, P. A., Verma, V. A., Paxton, T. J. & Pillow, T. H. Function-oriented synthesis, step economy, and drug design. Acc Chem Res 41, 40-49, doi:10.1021/ar700155p (2008).
21 Mediouni, S. et al. Didehydro-Cortistatin A Inhibits HIV-1 by Specifically Binding to the Unstructured Basic Region of Tat. mBio 10, doi:10.1128/mBio.02662-18 (2019).
22 Mediouni, S. et al. The Tat inhibitor didehydro-cortistatin A suppresses SIV replication and reactivation. Faseb j 33, 8280-8293, doi:10.1096/fj.201801165R (2019).
23 Sagnier, S. et al. Autophagy restricts HIV-1 infection by selectively degrading Tat in CD4+ T lymphocytes. J Virol 89, 615-625, doi:10.1128/jvi.02174-14 (2015).
24 Lata, S., Ali, A., Sood, V., Raja, R. & Banerjea, A. C. HIV-1 Rev downregulates Tat expression and viral replication via modulation of NAD(P)H:quinine oxidoreductase 1 (NQO1). Nat Commun 6, 7244, doi:10.1038/ncomms8244 (2015).
25 Ali, A. & Banerjea, A. C. Curcumin inhibits HIV-1 by promoting Tat protein degradation. Sci Rep 6, 27539, doi:10.1038/srep27539 (2016).
26 Zhang, L. et al. Modulation of the stability and activities of HIV-1 Tat by its ubiquitination and carboxyl-terminal region. Cell Biosci 4, 61, doi:10.1186/2045-3701-4-61 (2014).
27 Albornoz, N., Bustamante, H., Soza, A. & Burgos, P. Cellular Responses to Proteasome Inhibition: Molecular Mechanisms and Beyond. International Journal of Molecular Sciences 20, 3379 (2019).
28 Wan, Z. & Chen, X. Triptolide inhibits human immunodeficiency virus type 1 replication by promoting proteasomal degradation of Tat protein. Retrovirology 11, 88, doi:10.1186/s12977-014-0088-6 (2014).
29 Glickman, M. H. & Ciechanover, A. The ubiquitin-proteasome proteolytic pathway: destruction for the sake of construction. Physiol Rev 82, 373-428, doi:10.1152/physrev.00027.2001 (2002).
30 Asatsuma-Okumura, T., Ito, T. & Handa, H. Molecular mechanisms of cereblon-based drugs. Pharmacol Ther 202, 132-139, doi:10.1016/j.pharmthera.2019.06.004 (2019).
31 Li, J. et al. Long noncoding RNA NRON contributes to HIV-1 latency by specifically inducing tat protein degradation. Nat Commun 7, 11730, doi:10.1038/ncomms11730 (2016).
32 Ali, A., Farooqui, S. R. & Banerjea, A. C. The host cell ubiquitin ligase protein CHIP is a potent suppressor of HIV-1 replication. J Biol Chem 294, 7283-7295, doi:10.1074/jbc.RA118.007257 (2019).
33 Rice, A. P. & Carlotti, F. Structural analysis of wild-type and mutant human immunodeficiency virus type 1 Tat proteins. J Virol 64, 6018-6026 (1990).
34 Rice, A. P. & Carlotti, F. Mutational analysis of the conserved cysteine-rich region of the human immunodeficiency virus type 1 Tat protein. J Virol 64, 1864-1868 (1990).
35 Verhoef, K. & Berkhout, B. A second-site mutation that restores replication of a Tat-defective human immunodeficiency virus. J Virol 73, 2781-2789, doi:10.1128/JVI.73.4.2781-2789.1999 (1999).
36 Verhoef, K., Koper, M. & Berkhout, B. Determination of the minimal amount of Tat activity required for human immunodeficiency virus type 1 replication. Virology 237, 228-236, doi:S0042-6822(97)98786-9 [pii]10.1006/viro.1997.8786 (1997).
37 Xu, P. et al. Quantitative proteomics reveals the function of unconventional ubiquitin chains in proteasomal degradation. Cell 137, 133-145, doi:10.1016/j.cell.2009.01.041 (2009).
38 Boban, M., Ljungdahl, P. O. & Foisner, R. Atypical ubiquitylation in yeast targets lysine-less Asi2 for proteasomal degradation. J Biol Chem 290, 2489-2495, doi:10.1074/jbc.M114.600593 (2015).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 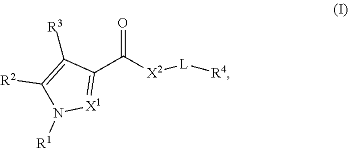
or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R² and R³ are independently hydrogen, halogen, substituted or unsubstituted carbocyclyl, substituted or unsubstituted heterocyclyl, substituted or unsubstituted aryl, or substituted or unsubstituted heteroaryl, or R² and R³, together with the atoms to which they are attached, are joined together to form a carbocyclic, heterocyclic, aryl, or heteroaryl ring, each of which may be optionally substituted;
L is substituted or unsubstituted alkylene or heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

2. The compound of claim 1, wherein R² and R³, together with the atoms to which they are attached, are joined together to form a substituted or unsubstituted carbocyclic, substituted or unsubstituted heterocyclic, substituted or unsubstituted aryl, or substituted or unsubstituted heteroaryl ring.

3. The compound of claim 1 or 2, wherein R² and R³, together with the atoms to which they are attached, are joined together to form a substituted or unsubstituted carbocyclic ring.

4. The compound of any one of claims 1-3, wherein R² and R³, together with the atoms to which they are attached, are joined together to form a substituted or unsubstituted cyclopentyl ring.

5. The compound of any one of claims 1-4, wherein the compound is of Formula (I-b): 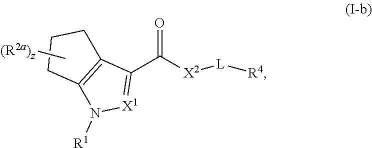
or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
L is a bond, substituted or unsubstituted alkylene, or substituted or unsubstituted heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{2a} is independently hydrogen, halogen, oxo, substituted or unsubstituted carbocyclyl, substituted or unsubstituted heterocyclyl, substituted or unsubstituted aryl, or substituted or unsubstituted heteroaryl; and
z is 1, 2, 3, 4, 5, or 6.

6. The compound of claim 5, wherein R^{2a} is H.

7. The compound of claim 5 or 6, wherein z is 1.

8. The compound of any one of claims 1-7, wherein the compound is of Formula (I-c): 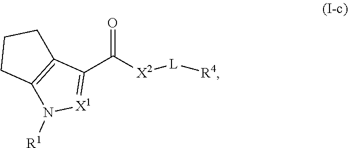
or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
L is a bond, substituted or unsubstituted alkylene, or substituted or unsubstituted heteroalkylene;
R⁴ is substituted or unsubstituted carbocyclyl, substituted or unsubstituted heterocyclyl, aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

9. The compound of any one of claims 1-3, wherein R² and R³, together with the atoms to which they are attached, are joined together to form a substituted or unsubstituted cyclohexyl ring.

10. The compound of any one of claims 1-3, or 9, wherein the compound is of Formula (I-d): 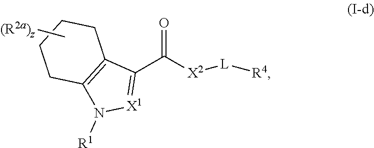
or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R² and R³ are independently hydrogen, halogen, carbocyclyl, heterocyclyl, aryl, or heteroaryl, or R² and R³, together with the atoms to which they are attached, are joined together to form a carbocyclic, heterocyclic, aryl, or heteroaryl ring;
L is substituted or unsubstituted alkylene or heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{2a} is hydrogen, halogen, oxo, hydroxy, optionally unsubstituted alkoxy, substituted or unsubstituted carbocyclyl, substituted or unsubstituted heterocyclyl, substituted or unsubstituted aryl, or substituted or unsubstituted heteroaryl; and
z is 1, 2, 3, 4, 5, or 6.

11. The compound of claim 10, wherein at least one instance of R^{2a} is oxo.

12. The compound of claim 10, wherein at least one instance of R^{2a} is hydroxy.

13. The compound of any one of claims 1-3, or 9-11, wherein the compound is of Formula (I-e): 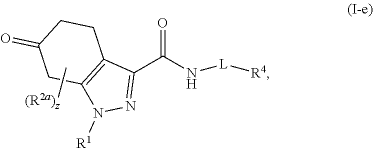
or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R² and R³ are independently hydrogen, halogen, substituted or unsubstituted carbocyclyl, substituted or unsubstituted heterocyclyl, substituted or unsubstituted aryl, or substituted or unsubstituted heteroaryl, or R² and R³, together with the atoms to which they are attached, are joined together to form a carbocyclic, heterocyclic, aryl, or heteroaryl ring, each of which may be optionally substituted;
L is a bond, substituted or unsubstituted alkylene, or substituted or unsubstituted heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{2a} is hydrogen, halogen, oxo, substituted or unsubstituted carbocyclyl, substituted or unsubstituted heterocyclyl, substituted or unsubstituted aryl, or substituted or unsubstituted heteroaryl; and
z is 1, 2, 3, 4, 5, or 6.

14. The compound of any one of claims 1-3, wherein R² and R³, together with the atoms to which they are attached, are joined together to form a substituted or unsubstituted aryl ring.

15. The compound of any one of claims 1-3, wherein R² and R³, together with the atoms to which they are attached, are joined together to form a substituted or unsubstituted phenyl ring.

16. The compound of any one of claims 1-3, or 14-15, wherein the compound is of Formula (I-f): 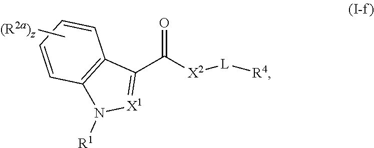
or a pharmaceutically acceptable salt, solvate, hydrate, polymorph, co-crystal, tautomer, stereoisomer, isotopically labeled compound, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R² and R³ are independently hydrogen, halogen, substituted or unsubstituted carbocyclyl, substituted or unsubstituted heterocyclyl, substituted or unsubstituted aryl, or substituted or unsubstituted heteroaryl, or R² and R³, together with the atoms to which they are attached, are joined together to form a carbocyclic, heterocyclic, aryl, or heteroaryl ring, each of which may be optionally substituted;
L is substituted or unsubstituted alkylene, or substituted or unsubstituted heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{2a} is hydrogen, halogen, oxo, substituted or unsubstituted carbocyclyl, substituted or unsubstituted heterocyclyl, substituted or unsubstituted aryl, or substituted or unsubstituted heteroaryl; and
z is 1, 2, 3, 4, 5, or 6.

17. The compound of any one of claims 10-16, wherein R^{2a} is H.

18. The compound of any one of claims 10-17, wherein z is 1.

19. The compound of any one of claims 1-18, wherein R¹ is substituted or unsubstituted aryl, or substituted or unsubstituted heteroaryl.

20. The compound of any one of claims 1-18, wherein R¹ is substituted or unsubstituted aryl.

21. The compound of any one of claims 1-18, wherein R¹ is substituted or unsubstituted phenyl.

22. The compound of any one of claims 1-21, wherein the compound is of Formula (I-g): 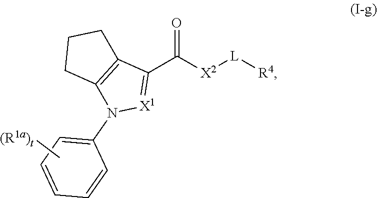
or a pharmaceutically acceptable salt, solvate, hydrate, polymorph, co-crystal, tautomer, stereoisomer, isotopically labeled compound, or prodrug thereof, wherein:
L is substituted or unsubstituted alkylene, or substituted or unsubstituted heteroalkylene;
R⁴ is substituted or unsubstituted carbocyclyl, substituted or unsubstituted heterocyclyl, substituted or unsubstituted aryl, or substituted or unsubstituted heteroaryl;
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
R^{1a} is hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; and
t is 1, 2, 3, 4, or 5.

23. The compound of any one of claims 1-22, wherein L is substituted or unsubstituted alkylene.

24. The compound of any one of claims 1-23, wherein L is substituted or unsubstituted C₁-C₆ alkylene.

25. The compound of any one of claims 1-24, wherein L is substituted or unsubstituted propylene.

26. The compound of any one of claims 1-25, wherein R⁴ is substituted or unsubstituted heterocyclyl.

27. The compound of any one of claims 1-26, wherein the compound is of Formula (I-h): 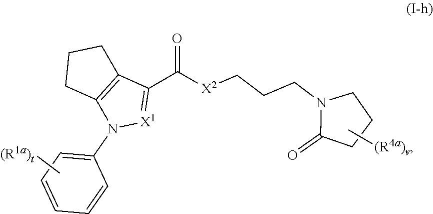
or a pharmaceutically acceptable salt, solvate, hydrate, polymorph, co-crystal, tautomer, stereoisomer, isotopically labeled compound, or prodrug thereof, wherein:
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{1a} is independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{4a} is independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl;
v is 1, 2, 3, 4, 5, or 6; and
t is 1, 2, 3, 4, or 5.

28. The compound of claim 27, wherein R^{4a} is H.

29. The compound of any one of claims 1-28, wherein the compound is of Formula (I-i): 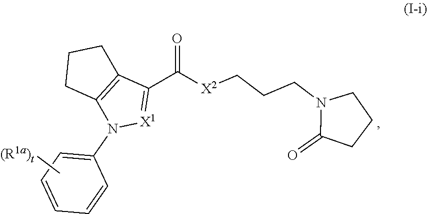
or a pharmaceutically acceptable salt, solvate, hydrate, polymorph, co-crystal, tautomer, stereoisomer, isotopically labeled compound, or prodrug thereof, wherein:
X¹ is —C(R^{a})═ or —N═;
X² is —C(R^{b})₂— or —N(R^{c})—;
R^{a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each of R^{b} is independently selected from hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
R^{c} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
each instance of R^{1a} is independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; and
t is 1, 2, 3, 4, or 5.

30. The compound of any one of claims 1-29, wherein X¹ is —N═.

31. The compound of any one of claims 1-30, wherein X² is —N(R^{c})—.

32. The compound of any one of claims 1-31, wherein R^{c} is hydrogen.

33. A compound of Formula (II′): 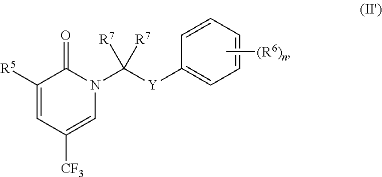
or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, hydroxy, optionally substituted alkoxy, substituted or unsubstituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, carbocyclyl, substituted or unsubstituted heterocyclyl, substituted or unsubstituted aryl, or substituted or unsubstituted heteroaryl;
Y is arylene, heteroarylene, —S(O₂)N(R^{d})—, —N(R^{d})C(═O)— or —C(═O)N(R^{d})—;
R^{d} is hydrogen, or substituted or unsubstituted alkyl; and
n is 1, 2, 3, 4, or 5.

34. A compound of Formula (II): 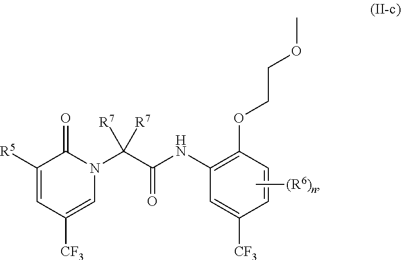
or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, hydroxy, optionally substituted alkoxy, substituted or unsubstituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, carbocyclyl, substituted or unsubstituted heterocyclyl, substituted or unsubstituted aryl, or substituted or unsubstituted heteroaryl;
Y is arylene, heteroarylene, —N(R^{d})C(═O)— or —C(═O)N(R^{d})—;
R^{d} is hydrogen, or substituted or unsubstituted alkyl; and
n is 1, 2, 3, 4, or 5.

35. The compound of claim 33 or 34, wherein Y is —C(═O)N(R^{d})—.

36. The compound of any one of claims 33-35, wherein R^{d} is hydrogen.

37. The compound of claim 33 or 34, wherein Y is heteroarylene.

38. The compound of any one of claims 33-36, wherein the compound is of Formula (II-a): 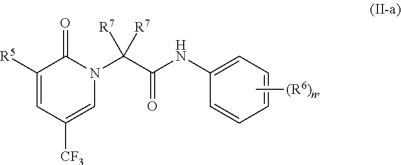
or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, optionally substituted alkoxy, substituted or unsubstituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
Y is substituted or unsubstituted arylene, substituted or unsubstituted heteroarylene, —N(R^{d})C(═O)— or —C(═O)N(R^{d})—; and
n is 1, 2, 3, 4, or 5.

39. The compound of any one of claims 33-38, wherein at least one instance of R⁶ is trifluoromethyl.

40. The compound of any one of claims 33-39, wherein at least one instance of R⁶ is optionally substituted alkoxy.

41. The compound of any one of claims 33-40, wherein the compound is of Formula (II-b): 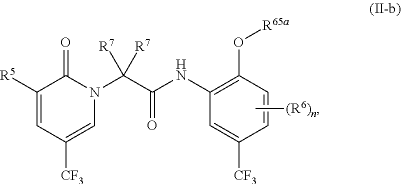
or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, optionally substituted alkoxy, substituted or unsubstituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R^{6a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
Y is substituted or unsubstituted arylene, substituted or unsubstituted heteroarylene, —N(R^{d})C(═O)— or —C(═O)N(R^{d})—; and
n is 1, 2, or 3.

42. The compound of claim 41, wherein R^{6a} is substituted or unsubstituted alkyl.

43. The compound of claim 41 or 42, wherein R^{6a} is substituted or unsubstituted C₁-C₆ alkyl.

44. The compound of claim 41 or 42, wherein R^{6a} is of the formula:

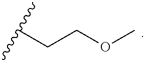 [SMILES: *CCOC]

45. The compound of any one of claims 33-44, wherein the compound is of Formula (II-c): 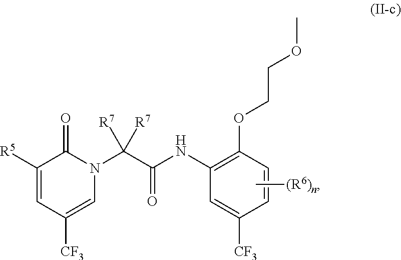
or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, a optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁷ is independently hydrogen, substituted or unsubstituted alkyl, optionally substituted alkoxy, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and
n is 1, 2, or 3.

46. The compound of any one of claims 33-45, wherein at least one instance of R⁷ is substituted or unsubstituted alkyl.

47. The compound of any one of claims 33-46, wherein at least on instance of R⁷ is substituted or unsubstituted C₁-C₆ alkyl.

48. The compound of any one of claims 33-47, wherein at least one instance of R⁷ is methyl.

49. The compound of any one of claims 33-45, wherein at least one instance of R⁷ is hydrogen.

50. The compound of any one of claims 33-47, wherein the compound is of Formula (II-d): 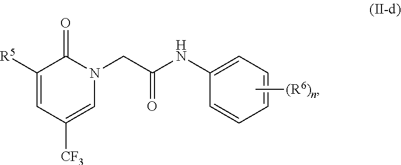
or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, hydroxy, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and
n is 1, 2, 3, 4, or 5.

51. The compound of any one of claims 33-50, wherein the compound is of Formula (II-e): 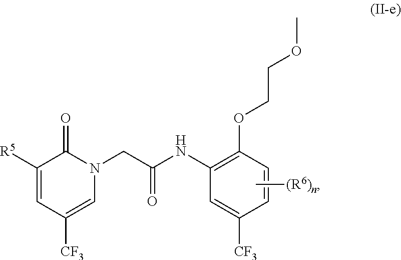
or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁶ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and
n is 1, 2, or 3.

52. The compound of any one of claims 33-49, wherein R⁶ is hydrogen.

53. The compound of any one of claims 33-52, wherein n is 1.

54. The compound of any one of claims 33-53, wherein n is 2.

55. The compound of any one of claims 33-54, wherein n is 3.

56. The compound of any one of claims 33-55, wherein the compound is of Formula (II-f): 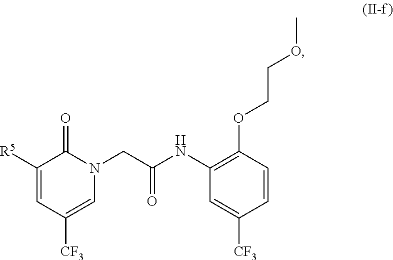
or a pharmaceutically acceptable salt, solvate, hydrate, polymorph, co-crystal, tautomer, stereoisomer, isotopically labeled compound, or prodrug thereof, wherein:
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

57. The compound of any one of claims 33-56, wherein R⁵ is halogen.

58. The compound of any one of claims 33-56, wherein R⁵ is chloro.

59. A compound of Formula (III′): 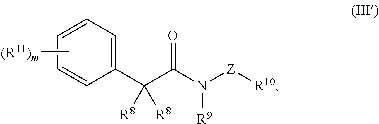
or a pharmaceutically acceptable salt, solvate, hydrate, polymorph, co-crystal, tautomer, stereoisomer, isotopically labeled compound, or prodrug thereof, wherein:
each R¹¹ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁸ is independently hydrogen, halogen, hydroxy, —N(R^{e})₂— substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
or one instance of R⁸ and R¹¹ is joined together with the atoms to which they are attached to form a 5 or 6 membered substituted or unsubstituted carbocyclic, substituted or unsubstituted heterocyclic, or substituted or unsubstituted heteroaryl ring;
each instance of R^{e} is independently hydrogen, substituted or unsubstituted alkyl, or substituted or unsubstituted acyl;
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
or one instance of R⁸ and R⁹ is joined together with the atoms to which they are attached to form a substituted or unsubstituted 5 membered heterocyclic ring;
R¹⁰ is substituted or unsubstituted carbocyclyl, substituted or unsubstituted heterocyclyl, substituted or unsubstituted aryl, or substituted or unsubstituted heteroaryl;
Z is substituted or unsubstituted alkylene, substituted or unsubstituted heteroalkylene, or substituted or unsubstituted heteroarylene; and
m is 1, 2, 3, 4, or 5.

60. A compound of Formula (III): 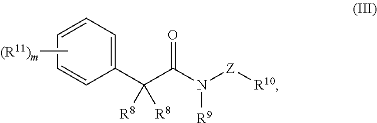
or a pharmaceutically acceptable salt, solvate, hydrate, polymorph, co-crystal, tautomer, stereoisomer, isotopically labeled compound, or prodrug thereof, wherein:
each R¹¹ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each R⁸ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
R¹⁰ is substituted or unsubstituted carbocyclyl, substituted or unsubstituted heterocyclyl, substituted or unsubstituted aryl, or substituted or unsubstituted heteroaryl;
Z is substituted or unsubstituted alkylene, or substituted or unsubstituted heteroalkylene; and
m is 1, 2, 3, 4, or 5.

61. The compound of claim 59 or 60, wherein at least one instance of R⁸ is substituted or unsubstituted aryl.

62. The compound of any one of claims 59-61, wherein at least one instance of R⁸ is substituted or unsubstituted phenyl.

63. The compound of any one of claims 59-62, wherein the compound of Formula (III) is of Formula (III-a): 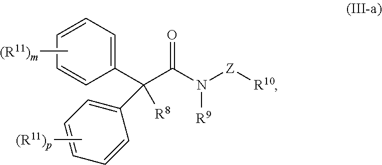
or a pharmaceutically acceptable salt, solvate, hydrate, polymorph, co-crystal, tautomer, stereoisomer, isotopically labeled compound, or prodrug thereof, wherein:
each R¹¹ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁸ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
R¹⁰ is carbocyclyl, heterocyclyl, aryl, or heteroaryl;
Z is substituted or unsubstituted alkylene or heteroalkylene;
p is 1, 2 3, 4, or 5; and
m is 1, 2, 3, 4, or 5.

64. The compound of any one of claims 59-63, wherein Z is substituted or unsubstituted alkylene.

65. The compound of any one of claims 59-64, wherein Z is substituted or unsubstituted C₁-C₆ alkylene.

66. The compound of any one of claims 59-65, wherein Z is substituted or unsubstituted propylene.

67. The compound of any one of claims 59-66, wherein R¹⁰ is substituted or unsubstituted heteroaryl.

68. The compound of any one of claims 59-67, wherein R¹⁰ is substituted or unsubstituted imidazolyl.

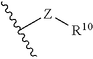

69. The compound of any one of claims 59-68, wherein is of the formula:

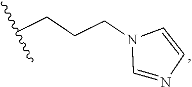 [SMILES: *CCCn1ccnc1]

70. The compound of any one of claims 59-69, wherein the compound of Formula (III) is of Formula (III-b): 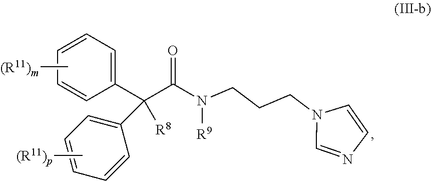
or a pharmaceutically acceptable salt, solvate, hydrate, polymorph, co-crystal, tautomer, stereoisomer, isotopically labeled compound, or prodrug thereof, wherein:
each R¹¹ is independently hydrogen, halogen, substituted or unsubstituted alkyl, optionally substituted alkoxy, optionally substituted acyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁸ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl; and
m is 1, 2, 3, 4, or 5.

71. The compound of any one of claims 59-70, wherein at least one instance of R¹¹ is hydrogen.

72. The compound of any one of claims 59-71, wherein the compound of Formula (III) is of Formula (III-c): 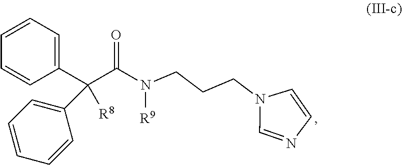
or a pharmaceutically acceptable salt, solvate, hydrate, polymorph, co-crystal, tautomer, stereoisomer, isotopically labeled compound, or prodrug thereof, wherein:
R⁸ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and
R⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

73. The compound of any one of claims 59-72, wherein at least one instance of R⁸ is substituted or unsubstituted alkyl.

74. The compound of any one of claims 59-73, wherein at least one instance of R⁸ is substituted or unsubstituted C₁-C₆ alkyl.

75. The compound of any one of claims 59-74, wherein at least one instance of R⁸ is methyl.

76. The compound of any one of claims 59-75, wherein at least one instance of R⁸ is hydrogen.

77. The compound of any one of claims 59-76, wherein R⁹ is hydrogen.

78. A compound selected from any one of those in Table 1, Table 2, or Table 3, or a pharmaceutically acceptable salt, solvate, hydrate, polymorph, co-crystal, tautomer, stereoisomer, isotopically labeled compound, or prodrug thereof.

79. The compound of any one of claims 1-78, wherein the compound binds preferentially to Tat residue Y26.

80. A pharmaceutical composition comprising the compound of any one of claims 1-79, or a pharmaceutically acceptable salt, solvate, hydrate, polymorph, co-crystal, tautomer, stereoisomer, isotopically labeled compound, or prodrug thereof, and an excipient.

81. The pharmaceutical composition of claim 80, further comprising an additional pharmaceutical agent.

82. The pharmaceutical composition of claim 81, wherein the additional pharmaceutical agent is an antiretroviral agent.

83. The pharmaceutical composition of claim 82, wherein the antiretroviral agent is a fusion inhibitor, a nucleoside/nucleotide reverse transcriptase inhibitor, a non-nucleoside reverse transcriptase inhibitor, a protease inhibitor, an integrase inhibitor, or a combination thereof.

84. A method of treating or preventing a disease in a subject in need thereof, comprising administering to the subject in need thereof the compound of any one of claims 1-79, or a pharmaceutically acceptable salt, solvate, hydrate, polymorph, co-crystal, tautomer, stereoisomer, isotopically labeled compound, or prodrug thereof, or the pharmaceutical composition of any one of claims 80-83.

85. The compound of any one of claims 1-79, or a pharmaceutically acceptable salt, solvate, hydrate, polymorph, co-crystal, tautomer, stereoisomer, isotopically labeled compound, or prodrug thereof, or the pharmaceutical composition of any one of claims 80-83, for use in treating or preventing a disease in a subject in need thereof.

86. The compound of any one of claims 1-79, or a pharmaceutically acceptable salt, solvate, hydrate, polymorph, co-crystal, tautomer, stereoisomer, isotopically labeled compound, or prodrug thereof, or the pharmaceutical composition of any one of claims 80-83, for use in the manufacture of a medicament for treatment or prevention of a disease in a subject in need thereof.

87. The method of claim 84, or compound for use of claims 85 or 86, wherein the disease is associated with human immunodeficiency virus type 1 (HIV-1) Tat protein.

88. The method of claim 84, or compound for use of any one of claims 85-87, wherein the disease is a viral disease.

89. The method of claim 84, or compound for use of any one of claims 85-88, wherein the disease is HIV-1.

90. The method of claim 84, or compound for use of any one of claims 85-89, further comprising administering to the subject in need thereof an additional pharmaceutical agent.

91. The method of claim 84, or compound for use of any one of claims 85-90, further comprising administering to the subject in need thereof an antiretroviral agent.

92. The method or compound for use of claim 91, wherein the antiretroviral agent is a fusion inhibitor, a nucleoside/nucleotide reverse transcriptase inhibitor, a non-nucleoside reverse transcriptase inhibitor, a protease inhibitor, an integrase inhibitor, or a combination thereof.

93. A method of binding HIV-1 Tat protein in a subject in need thereof or in a cell, tissue, or biological sample, comprising administering to the subject in need thereof or contacting the cell, tissue, or biological sample with an effective amount of the compound of any one of claims 1-79, or a pharmaceutically acceptable salt, solvate, hydrate, polymorph, co-crystal, tautomer, stereoisomer, isotopically labeled compound, or prodrug thereof, or the pharmaceutical composition of any one of claims 80-83;
wherein the cell, tissue, or biological sample is in vivo.

94. A method of binding HIV-1 Tat protein in a subject in need thereof or in a cell, tissue, or biological sample, comprising administering to the subject in need thereof or contacting the cell, tissue, or biological sample with an effective amount of the compound of any one of claims 1-79, or a pharmaceutically acceptable salt, solvate, hydrate, polymorph, co-crystal, tautomer, stereoisomer, isotopically labeled compound, or prodrug thereof, or the pharmaceutical composition of any one of claims 80-83;
wherein the cell, tissue, or biological sample is in vitro.

95. A method of inhibiting HIV-1 Tat protein in a subject in need thereof or in a cell, tissue, or biological sample, comprising administering to the subject in need thereof or contacting the cell, tissue, or biological sample with an effective amount of the compound of any one of claims 1-79, or a pharmaceutically acceptable salt, solvate, hydrate, polymorph, co-crystal, tautomer, stereoisomer, isotopically labeled compound, or prodrug thereof, or the pharmaceutical composition of any one of claims 80-83;
wherein the cell, tissue, or biological sample is in vivo.

96. A method of inhibiting HIV-1 Tat protein in a subject in need thereof or in a cell, tissue, or biological sample, comprising administering to the subject in need thereof or contacting the cell, tissue, or biological sample with an effective amount of the compound of any one of claims 1-79, or a pharmaceutically acceptable salt, solvate, hydrate, polymorph, co-crystal, tautomer, stereoisomer, isotopically labeled compound, or prodrug thereof, or the pharmaceutical composition of any one of claims 80-83;
wherein the cell, tissue, or biological sample is in vitro.

97. A method of promoting degradation of HIV-1 Tat protein in a subject in need thereof or in a cell, tissue, or biological sample, comprising administering to the subject in need thereof or contacting the cell, tissue, or biological sample with an effective amount of the compound of any one of claims 1-79, or a pharmaceutically acceptable salt, solvate, hydrate, polymorph, co-crystal, tautomer, stereoisomer, isotopically labeled compound, or prodrug thereof, or the pharmaceutical composition of any one of claims 70-83;
wherein the cell, tissue, or biological sample is in vivo.

98. A method of promoting degradation of HIV-1 Tat protein in a subject in need thereof or in a cell, tissue, or biological sample, comprising administering to the subject in need thereof or contacting the cell, tissue, or biological sample with an effective amount of the compound of any one of claims 1-79, or a pharmaceutically acceptable salt, solvate, hydrate, polymorph, co-crystal, tautomer, stereoisomer, isotopically labeled compound, or prodrug thereof, or the pharmaceutical composition of any one of claims 80-83;
wherein the cell, tissue, or biological sample is in vitro.

99. A kit comprising the compound of any one of claims 1-79, or a pharmaceutically acceptable salt, solvate, hydrate, polymorph, co-crystal, tautomer, stereoisomer, isotopically labeled compound, or prodrug thereof, or the pharmaceutical composition of any one of claims 80-83; and
instructions for using the compound of any one of claims 1-79, or a pharmaceutically acceptable salt, solvate, hydrate, polymorph, co-crystal, tautomer, stereoisomer, isotopically labeled compound, or prodrug thereof, or the pharmaceutical composition of any one of claims 80-83.
